(19) United States
(12) Patent Application Publication
(10) Pub. No.: US 2020/0407700 A1
(43) Pub. Date: Dec. 31, 2020

(54) COMPOSITIONS OF PARP14 MODULATORS AND/OR MUTANTS AND THERAPEUTIC USE THEREOF

(71) Applicant: President and Fellows of Harvard College, Cambridge, MA (US)

(72) Inventors: David A. Sinclair, Chestnut Hill, MA (US); Michael B. Schultz, Boston, MA (US)

(21) Appl. No.: 16/975,955

(22) PCT Filed: Feb. 25, 2019
(86) PCT No.: PCT/US19/19426

§ 371 Date: Aug. 26, 2020

Related U.S. Application Data
(60) Provisional application No. 62/740,088, filed on Oct. 2, 2018.
(60) Provisional application No. 62/635,325, filed on Feb. 26, 2018.

(51) Int. Cl. C12N 9/10; A61P 39/06; A61K 38/45; A61P 29/00; A61K 31/166; A61K 31/473; A61K 31/4178

(52) U.S. Cl. CPC C12N 9/1077; A61P 39/06; C12Y 204/0203; A61K 31/4178; A61P 29/00; A61K 31/166; A61K 31/473; A61K 38/45

(57) ABSTRACT

The invention provides compositions comprising agents which modulate poly(ADP-ribose)polymerase 14 (PARP14), or compositions comprising PARP14 mutants. Such compositions can modulate the levels of nicotinamide adenine dinucleotide (NAD+) and are useful in methods for treating or preventing cancer, aging, aging-related disorders, cell death, radiation damage, radiation exposure, disorders associated with inflammation, among others. Such compositions and methods may also improve DNA repair, cell proliferation, cell survival, modulate inflammatory response, among others, and may increase the life span of a cell or protect it against certain stresses, apoptosis, among others.

### RELATED U.S. APPLICATIONS

This application claims priority to U.S. Provisional Application 62/635,325 filed Feb. 26, 2018, and U.S. Provisional Application 62/740,088, filed Oct. 2, 2018, each of which is incorporated herein by reference in its entirety.

### STATEMENT REGARDING FEDERALLY SPONSORED RESEARCH

This invention was made with Government support of Grant No. AG028730, awarded by the National Institutes of Health. The Government has certain rights in this invention.

### BACKGROUND OF THE INVENTION

NAD⁺ levels are important for many cellular functions, including energy metabolism, DNA repair, and epigenetic maintenance. For example, NAD⁺ levels decline with age (Gomes et al. Cell 155 (7):1624-38 (2013)) and are raised by calorie restriction and exercise in humans and in rodents. Interventions that raise NAD⁺ (e.g., calorie restriction and exercise) have been shown to reduce cancer risk and prevent tumor growth (Meynet et al. Trends Mol Med 20 (8):419-27 (2014); Lagopoulos et al. Carcinogenesis, 8 (1):33-7 (1987). The NAD⁺ precursors nicotinamide mononucleotide (NMN) and nicotinamide riboside (NR) have been shown to improve metabolism and reverse aspects of ageing in elderly mice (Gomes et al. Cell 155 (7):1624-38 (2013)). Understanding the mechanism and interplay of NAD⁺ metabolism in cellular functions, including energy metabolism, DNA repair, apoptosis, and inflammatory responses, may shed light on the regulation of these processes, and provide novel methods and therapies for aging or aging-related disorders, inflammation associated disorders, among other diseases.

### SUMMARY OF THE INVENTION

The present invention is based, at least in part, on the discovery that PARP14 is a major regulator of NAD⁺ levels.

One aspect of the invention relates to a method for treating or preventing aging, or an aging-related disorder, in a subject in need thereof comprising administering to the subject an effective amount of: (a) an agent that modulates the level of, activity of, or expression of a poly(ADP-ribose)polymerase 14 (PARP14), or fragment thereof, or a nucleic acid encoding same; (b) a PARP14 mutant, or fragment thereof, or a nucleic acid encoding same; or (c) both (a) and (b); to thereby modulate the levels of nicotinamide adenine dinucleotide (NAD⁺) in the subject.

Another aspect of the invention relates to a method for treating or preventing a disorder associated with inflammation in a subject in need thereof comprising administering to the subject an effective amount of: (a) an agent that modulates the level of, activity of, or expression of a PARP14, or a fragment thereof, or a nucleic acid encoding same; (b) a PARP14 mutant, or fragment thereof, or a nucleic acid encoding same; or (c) both (a) and (b); to thereby modulate the levels of NAD⁺ in the subject.

Another aspect of the invention relates to a method of modulating an inflammatory response in a subject in need thereof comprising administering to the subject an effective amount of: (a) an agent that modulates the level of, activity of, or expression of a PARP14, or a fragment thereof, or a nucleic acid encoding same; (b) a PARP14 mutant, or fragment thereof, or a nucleic acid encoding same; or (c) both (a) and (b); to thereby modulate the levels of NAD⁺ in the subject.

Another aspect of the invention relates to a method for increasing stress resistance of a cell comprising introducing into the cell: (a) an agent that modulates the level of, activity of, or expression of a PARP14, or a fragment thereof, or a nucleic acid encoding same; (b) a PARP14 mutant, or fragment thereof, or a nucleic acid encoding same; or (c) both (a) and (b); to thereby modulate the levels of NAD⁺ in the cell.

In some embodiments of any of the aforementioned methods, the cell is a mammalian cell, yeast cell, fungal cell, plant cell, or microbial cell.

In some embodiments of any of the aforementioned methods, the agent inhibits the level of, activity of, or expression of the PARP14, or a fragment thereof, or a nucleic acid encoding same.

In some embodiments of any of the aforementioned methods, the agent inhibits the level of, activity of, or expression of the PARP14, or homologs thereof, as set forth in Table 1.

In some embodiments of any of the aforementioned methods, the PARP14 mutant comprises at least one substitution, mutations, insertion, deletion, or combination thereof, in Macro Domain 1 as set forth in Table 1 or 2.

In some embodiments of any of the aforementioned methods, the PARP14 mutant comprises at least two, three, four, five, six, seven, eight, nine, ten, or more substitution, mutations, insertion, deletion, or combinations thereof, in Macro Domain 1 as set forth in Table 1 or 2.

In some embodiments of any of the aforementioned methods, the PARP14 mutant comprises at least one substitution, mutations, insertion, or deletion of a phosphorylation site as set forth in Table 1 or 3.

In some embodiments of any of the aforementioned methods, the PARP14 mutant is biologically inactive or functionally defective.

In some embodiments of any of the aforementioned methods, the PARP14 mutant lacks NADase activity, ADP-ribose hydrolase, mono(ADP-ribosyl)transferase activity, or poly(ADP-ribosyl)transferase activity, or combinations thereof.

In some embodiments of any of the aforementioned methods, the agent is a PARP14 inhibitor.

In some embodiments of any of the aforementioned methods, the PARP14 inhibitor is a pan-PARP inhibitor.

In some embodiments of any of the aforementioned methods, the pan-PARP inhibitor is selected from 3-aminobenzamide, KU0058948, BGB-290, Olaparib, ABT-888, CEP-9722, DPQ, NU1025, EB-47, E7016, DiQ, DR2313, 4-ANI, ISQ, 3-hydroxybenzamide, CNQ, 3-AB, PJ34, DPQ, INH2BP, Iniparib, Niraparib (MK-4827), 6(5H)-phenanthridinone, 3-methyl-5-AIQ, Talazoparib, TIQ-A, XAV939, Veliparib, or Rucaparib, or combination thereof.

In some embodiments of any of the aforementioned methods, the pan-PARP inhibitor is 3-aminobenzimide or PJ-34.

In some embodiments of any of the aforementioned methods, the PARP14 inhibitor is a Macro Domain 1 inhibitor.

In some embodiments of any of the aforementioned methods, the Macro Domain 1 inhibitor is selected from NCI-61610 (C₃₄H₂₄N₆O₂), NCI-25457 (C₂₄H₁₆N₂O), NCI-345647_a (C₃₀H₂₆O₁₀), NCI-670283 (C₂₅₄H₂₄O₂), or NCI_127133 (C₂₇H₁₈N₂O₄), or combinations thereof.

In some embodiments of any of the aforementioned methods, the Macro Domain 1 inhibitor is selected from NSC-61610 or NSC-127-133.

In some embodiments of any of the aforementioned methods, the levels of NAD⁺ are increased.

In some embodiments of any of the aforementioned methods, the agent or PARP14 mutant blocks the fall of NAD⁺levels in the subject.

In some embodiments of any of the aforementioned methods, the agent or PARP14 mutant increases the level or activity of an enzyme involved in NAD⁺ biosynthesis, an enzymatically active fragment of such an enzyme, a nucleic acid encoding an enzyme involved in NAD⁺ biosynthesis, or an enzymatically active fragment of such a nucleic acid.

In some embodiments of any of the aforementioned methods, the enzyme is selected from mononucleotide adenylyl transferasel (NMNAT1), NMNAT2, NMNAT3, or nicotinamide phosphoribosyl transferase (NAMPT or NAMPRT).

In some embodiments of any of the aforementioned methods, the inflammasome activation is suppressed.

In some embodiments of any of the aforementioned methods, inflammation is decreased.

In some embodiments of any of the aforementioned methods, an inflammatory response is depressed or suppressed.

In some embodiments, the aging-related disorder is selected from the group consisting of Alzheimer's disease, diabetes mellitus, heart disease, obesity, osteoporosis, Parkinson's disease, stroke, amniotropic lateral sclerosis, arthritis, atherosclerosis, cachexia, cancer, cardiac hypertrophy, cardiac failure, cardiac hypertrophy, cardiovascular disease, cataracts, colitis, chronic obstructive pulmonary disease, dementia, diabetes mellitus, frailty, heart disease, hepatic steatosis, high blood cholesterol, high blood pressure, Huntington' s disease, hyperglycemia, hypertension, infertility, inflammatory bowel disease, insulin resistance disorder, lethargy, metabolic syndrome, muscular dystrophy, multiple sclerosis, neuropathy, nephropathy, obesity, osteoporosis, Parkinson' s disease, psoriasis, retinal degeneration, sarcopenia, sleep disorders, sepsis, and stroke.

In some embodiments, the disorder associated with inflammation is selected from the group consisting of: septic shock, obesity-related inflammation, Parkinson's Disease, Crohn's Disease, Alzheimer's Disease (AD), cardiovascular disease (CVD), inflammatory bowel disease (IBD), chronic obstructive pulmonary disease, an allergic reaction, an autoimmune disease, blood inflammation, joint inflammation, arthritis, asthma, ulcerative colitis, hepatitis, psoriasis, atopic dermatitis, pemphigus, glomerulonephritis, atherosclerosis, sarcoidosis, rheumatoid arthritis, psoriatic arthritis, ankylosing spondylitis, Wegner's syndrome, Goodpasture's syndrome, giant cell arteritis, polyarteritis nodosa, idiopathic pulmonary fibrosis, acute lung injury, post-influenza pneumonia, SARS, tuberculosis, malaria, sepsis, cerebral malaria, Chagas disease, schistosomiasis, bacterial and viral meningitis, cystic fibrosis, multiple sclerosis, encephalomyelitis, sickle cell anemia, pancreatitis, transplantation, systemic lupus erythematosis, autoimmune diabetes, thyroiditis, and radiation pneumonitis, respiratory inflammation, and pulmonary inflammation.

In some embodiments of any of the aforementioned methods, the agent or PARP14 mutant is administered to the subject at a dose of between 0.5-5 grams per day.

In some embodiments of any of the aforementioned methods, the agent or the PARP14 mutant is administered in a pharmaceutically effective amount.

In some embodiments of any of the aforementioned methods, the pharmaceutically effective amount is provided as a pharmaceutical composition in combination with a pharmaceutically-acceptable excipient, diluent, or carrier.

In some embodiments of any of the aforementioned methods, the a) agent is administered simultaneously as the PARP14 mutant, b) agent is administered in combination with PARP14 mutant, c) agent is administered prior to administering the PARP14 mutant, or d) agent is administered subsequently to administering the PARP14 mutant.

In some embodiments of any of the aforementioned methods, the subject is a mammal or non-mammal.

In some embodiments of any of the aforementioned methods, the subject is a human.

Another aspect of the invention relates to an agent or PARP14 mutant that increases the level of NAD⁺ for use in treating or preventing aging, or an aging-related disorder.

Another aspect of the invention relates to an agent or PARP14 mutant that increases the level of NAD⁺ for use in treating or preventing a disorder associated with inflammation.

Another aspect of the invention relates to an agent or PARP14 mutant that increases the level of NAD⁺ for use in modulating an inflammatory response.

Another aspect of the invention relates to an agent or PARP14 mutant that increase the level of NAD⁺ for use in increasing stress resistance of a cell.

In some embodiments of any of the aforementioned agents, the agent inhibits the level of, activity of, or expression of the PARP14, or a fragment thereof, or a nucleic acid encoding same.

In some embodiments of any of the aforementioned agents, the agent inhibits the level of, activity of, or expression of the PARP14, or homologs thereof, as set forth in Table 1.

In some embodiments of any of the aforementioned agents, the agent is a PARP14 inhibitor.

In some embodiments of any of the aforementioned agents, the PARP14 inhibitor is a pan-PARP inhibitor.

In some embodiments of any of the aforementioned agents, the pan-PARP inhibitor is selected from 3-aminobenzamide, KU0058948, BGB-290, Olaparib, ABT-888, CEP-9722, DPQ, NU1025, EB-47, E7016, DiQ, DR2313, 4-ANI, ISQ, 3-hydroxybenzamide, CNQ, 3-AB, PJ34, DPQ, INH2BP, Iniparib, Niraparib (MK-4827), 6(5H)-phenanthridinone, 3-methyl-5-AIQ, Talazoparib, TIQ-A, XAV939, Veliparib, or Rucaparib, or combination thereof.

In some embodiments of any of the aforementioned agents, the pan-PARP inhibitor is 3-aminobenzimide or PJ-34.

In some embodiments of any of the aforementioned agents, the PARP14 inhibitor is a Macro Domain 1 inhibitor.

In some embodiments of any of the aforementioned agents, the Macro Domain 1 inhibitor is selected from NCI-61610 (C₃₄H₂₄N₆O₂), NCI-25457 (C₂₄H₁₆N₂O), NCI-345647_a (C₃₀H₂₆O₁₀), NCI-670283 (C₂₅₄H₂₄O₂), or NCI_127133 (C₂₇H₁₈N₂O₄), or combinations thereof.

In some embodiments of any of the aforementioned agents, the Macro Domain 1 inhibitor is selected from NSC-61610 or NSC-127-133.

In some embodiments of any of the aforementioned PARP14 mutant, said mutant comprises at least one substitution, mutation, insertion, deletion, or combination thereof, in Macro Domain 1 as set forth in Table 1 or 2.

In some embodiments of any of the aforementioned PARP14 mutant, said mutant comprises at least two, three, four, five, six, seven, eight, nine, ten, or more substitutions, mutations, insertions, deletions, or combination thereof, in Macro Domain 1 as set forth in Table 1 or 2.

In some embodiments of any of the aforementioned PARP14 mutant, said mutant comprises at least one substitution, mutations, insertion, or deletion of a phosphorylation site as set forth in Table 1 or 3.

In some embodiments of any of the aforementioned PARP14 mutant, said mutant is biologically inactive or functionally defective.

In some embodiments of any of the aforementioned PARP14 mutant, said mutant lacks NADase activity, ADP-ribose hydrolase, mono(ADP-ribosyl)transferase activity, or poly(ADP-ribosyl)transferase activity, or combinations thereof.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 contains 5 panels, 1A-1E, depicting that NAD⁺ levels fluctuate in response to LPS. FIG. 1A depicts NAD⁺ levels over a 24-hour LPS time course in bone marrow derived macrophages (BMDMs). FIG. 1B depicts NADH levels over a 24-hour LPS time course in BMDMs. FIG. 1C depicts localization of an NAD⁺-sensor to the nucleus, cytoplasm, or mitochondria, and fluorescence in response to LPS, in RAW264.7 immortalized macrophages. FIG. 1D shows an overview of NAD⁺ metabolic pathways. FIG. 1E shows a mass-spec metabolomics of major NAD-metabolites at 0, 6, and 24 hours of LPS treatment.

FIG. 2 contains 13 panels, 2A-2M, depicting that PARP14 is necessary for LPS-induced NAD⁺ destruction. FIG. 2A-2C show NAD⁺ levels in response to LPS in wild-type or (FIG. 2A) CD38KO, (FIG. 2B) SARM1KO, and (FIG. 2C) PARP1KO BMDMs. FIG. 2D shows RNA-levels of enzymes relevant to NAD⁺ metabolism in BMDMs after 0, 6, or 24 hours of LPS treatment. FIG. 2E shows NAD⁺ levels in BMDMs after 6 hours of 3-aminobenzimide, PJ-34, or (FIG. 2F) OUL35 (Venkannagari et al. Cell Chemical Biology 23:1251-1260 (2016)) treatment, in the absence or presence of LPS. FIG. 2G shows ADP-ribosyltransferase activity of BMDMs after treatment with LPS. FIG. 2H shows NAD⁺ levels of LPS-treated cells relative to untreated cells in WT and PARP7, 9, 10, 11, 12, and 14 knockout RAW264.7 cell lines. FIG. 21 shows PARP14 levels in WT and knockout cell lines. FIG. 2J shows PARP14 levels in BMDMs in response to LPS. FIGS. 2K and 2L show NAD⁺ (2K) and NADH levels (2L) in PARP14KO BMDMs. FIG. 2M shows Western blot analysis of PARP14 levels in WT and KO.

FIG. 3 contains 6 panels, 3A-3F, depicting PARP14 catalytic mechanism. FIG. 3A shows in vitro NADase activity of recombinant CD38 and PARP14. FIG. 3B shows in vitro ADP-ribosyltransferase activity of recombinant PARP14. FIG. 3C shows PARP14 domains (adapted from Daugherty, PLOS Genetics, 10 (5): e1004403 (2014)). FIG. 3D shows a comparison of human and mouse (green boxes) PARP14 Macro domain 1 sequence to that of known catalytically active and inactive Macro domains (adapted from Jankevicius, Nat. Struc. and Mol. Biol., 20 (4):508-14 (2013)). FIG. 3E depicts NAD⁺ levels after treatment with NSC-61610. FIG. 3F depicts NAD⁺ levels after treatment with NSC-127133 and LPS.

FIG. 4 contains 5 panels, 4A-4E, depicting that NAD⁺ decline promotes inflammasome activation. FIG. 4A-4D show secretion of (FIG. 4A) IL-1β, (FIG. 4B) IL-18, (FIG. 4C) IL-6, and (FIG. 4D) TNF in response to LPS and nigericin, without and with NMN. FIG. 4E shows Caspase 1 cleavage (Casp-1 p20) in the presence of PJ34 and OUL35.

FIG. 5 depicts that NAD⁺ levels decline in aged peritoneal macrophages.

FIG. 6 depicts the phosphorylation site between the Macro domain 3 and the PARP domain. The phosphorylation site is likely important for PARP14 NADase activity.

Note that for every figure containing a histogram, the bars from left to right for each discreet measurement correspond to the figure boxes from top to bottom in the figure legend as indicated.

FIG. 7 contains five panels, 7A-7E, showing NAD⁺ levels fluctuate in response to TLR activation. Part 7A shows NAD⁺ and NADH levels over a 48-hour LPS time course in BMDMs (Part 7B-C) NAD⁺ levels after 6-hour treatment with TLR activators P3CSK4 and palmitic acid (PA). 7D-E shows NAD⁺ and NADH levels over a 48-hour time course in BMDMs with IFNg and IL-4.

FIG. 8 contains two panels, 8A-8B, and shows PARP14 levels in BMDMs in response to LPS (Part A). Comparison of NAD⁺ levels over a 24-hour LPS time course in SARM1 KO, PARP1 KO, CD38 KO and PARP14 KO BMDMs, with relevant WT controls (Part B).

FIG. 9 contains three panels, 9A-9C, and shows PARP14 is sufficient to lower NAD+ levels (Part A). Part B shows NAD+ levels and PARP14 protein levels in 293T cells overexpressing PARP14. Part C shows NAD⁺ levels in 293T cells transfected with GFP, PARP14, and point mutants of PARP14 in macro domain 1 (MAC1) and the PARP catalytic domain (CAT).

FIG. 10 contains three panels, 10A-C, and shows a comparison of NAD precursors. Part A and B shows NMN, NR, NAR, and NRH's effect on NAD⁺ levels in BMDMs in the absence and presence of LPS, after 6 hours. Part C shows the structure of NRH.

FIG. 11 contains five panels, 11A-F and shows NAD+ decline and inflammatory response. Parts A shows experimental design. Part B shows NAD+ repletion restores poly-ADP-riboslyation level. Part C shows NAD+ repletion may suppress inflammasome activation. Part D-F shows TNFa, IL-6, and IL-1b RNA levels.

FIG. 12 has fifteen panels, 12A-O, and shows NAD+ levels and metabolomics. Part A-B shows a plot of log2 fold changes in metabolites of LPS vs CTL (X-axis) and Drug+LPS vs LPS (Y-axis). Part C shows the top 25 metabolites that changed with LPS and in response to PARPi and NRH. Part D shows pathways involved in the top 50 metabolites from previous analysis. Part E shows purine metabolism. Parts F-N shows purine metabolite levels. Part O shows preliminary measurement of ROS levels after 30 min nigericin treatment.

FIG. 13 has seven panels, 13A-G, and shows LPS-induced sepsis. Parts A-D shows PARP14 levels in tissues 6 hours after I.P. injection of LPS. Part E shows NAD+ levels in the spleen after 6-hour I.P. injection of LPS with vehicle, PARPi, or NRH. Part F shows survival after I.P. injection of LPS with vehicle, PARPi, or NRH. Part G shows survival after LPS I.P. injection in PARP14 WT and KO male mice.

FIG. 14 shows PARP14 and aging, and shows NAD⁺ levels in peritoneal macrophages from young, middle-aged, and old mice.

FIG. 15 shows PARP14 levels increase in aging spleen. Spleens were collected from 3 month old and 24 month old mice. PARP14 protein levels were assessed by Western blot, relative to beta actin.

FIG. 16 shows OUL raises NAD⁺ levels in aged spleens. 27 month old mice were injected intraperitoneally with 20 mg/kg of OUL35 dissolved in PBS with 40% HP-beta-cyclodextrin and 1% carboxymethylcellulose. Spleens were collected 2 hours post-injection and assayed for NAD+ levels.

### DETAILED DESCRIPTION OF THE INVENTION

### Definitions

As used herein, the following terms and phrases shall have the meanings set forth below. Unless defined otherwise, all technical and scientific terms used herein have the same meaning as commonly understood to one of ordinary skill in the art to which this invention belongs.

The singular forms “a,” “an,” and “the” include plural reference unless the context clearly dictates otherwise.

Recitation of ranges of values herein are merely intended to serve as a shorthand method of referring individually to each separate value falling within the range, unless otherwise indicated herein, each individual value is incorporated into the specification as if it were individually recited herein. Ranges may be expressed herein as from “about” (or “approximate”) one particular value, and/or to “about” (or “approximate”) another particular value. When such a range is expressed, another embodiment includes from the one particular value and/or to the other particular value. Similarly, when values are expressed as approximations, by use of the antecedent “about” or “approximate” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that is “less than or equal to the value” or “greater than or equal to the value” possible ranges between these values are also disclosed, as appropriately understood by the skilled artisan. For example, if the value “10” is disclosed the “less than or equal to 10” as well as “greater than or equal to 10” is also disclosed.

All methods described herein can be performed in any suitable order unless otherwise indicated herein or otherwise clearly contradicted by context. Further, all methods described herein and having more than one step can be performed by more than one person or entity. Thus, a person or an entity can perform step (a) of a method, another person or another entity can perform step (b) of the method, and a yet another person or a yet another entity can perform step (c) of the method, etc. The use of any and all examples, or exemplary language (e.g. “such as”) provided herein is intended merely to better illuminate the invention and does not pose a limitation on the scope of the invention otherwise claimed.

Units, prefixes, and symbols are denoted in their Systeme International de Unites (SI) accepted form. Unless otherwise indicated, nucleic acids are written left to right in 5′ to 3′ orientation; amino acid sequences are written left to right in amino to carboxy orientation.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

The headings used herein are for organizational purposes only and are not meant to be used to limit the scope of the description or the claims, which can be had by reference to the specification as a whole. Accordingly, the terms defined immediately below are more fully defined by reference to the specification in its entirety.

Illustrations are for the purpose of describing a preferred embodiment of the invention and are not intended to limit the invention thereto.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used herein, the term “administering” means the actual physical introduction of a composition into or onto (as appropriate) a host or cell. Any and all methods of introducing the composition into the host or cell are contemplated according to the invention; the method is not dependent on any particular means of introduction and is not to be so construed. Means of introduction are well-known to those skilled in the art, and also are exemplified herein.

As used herein, administration “in combination” refers to both simultaneous and sequential administration of two or more compositions. Concurrent or combined administration, as used herein, means that two or more compositions are administered to a subject either (a) simultaneously, or (b) at different times during the course of a common treatment schedule. In the latter case, the two or more compositions are administered sufficiently close in time to achieve the intended effect.

As used herein, “aging-related disorders”, include but not limited to, Alzheimer's disease, diabetes mellitus, heart disease, obesity, osteoporosis, Parkinson's disease, stroke, amniotropic lateral sclerosis, arthritis, atherosclerosis, cachexia, cancer, cardiac hypertrophy, cardiac failure, cardiac hypertrophy, cardiovascular disease, cataracts, colitis, chronic obstructive pulmonary disease, dementia, diabetes mellitus, frailty, heart disease, hepatic steatosis, high blood cholesterol, high blood pressure, Huntington' s disease, hyperglycemia, hypertension, infertility, inflammatory bowel disease, insulin resistance disorder, lethargy, metabolic syndrome, muscular dystrophy, multiple sclerosis, neuropathy, nephropathy, obesity, osteoporosis, Parkinson' s disease, psoriasis, retinal degeneration, sarcopenia, sleep disorders, sepsis, and/or stroke.

As used herein, the term “DNA damage” mean a change in a nucleic acid sequence (in comparison to a wildtype or normal nucleic acid sequence) that alters or eliminates the function of an encoded polypeptide, that alters or eliminates the amount of an encoded polypeptide produced, or that alters or eliminates a regulatory function of the nucleic acid having acquired a mutation. Mutations or DNA damage include, but are not limited to, point mutations, deletions, insertions, inversions, duplications, single-stranded DNA breaks, double-stranded DNA breaks, and DNA lesions as known in the art.

As used herein, the term “DNA repair deficiency disorder” refers to a disorder in a subject in which one or more components of the DNA repair pathway(s) is underexpressed, mutated, or less functional than the same component in a wild-type organism. A DNA repair deficiency disorder may refer to a subject in which at least a cell has a mutation. Examples of DNA repair deficiency disorders include, but are not limited to, Ataxia Telangiectasia (A-T), Xeroderma Pigmentosum (XP), Fanconi's Anemia (FA), Li Fraumeni syndrome, Nijmegen breakage syndrome (NBS), A-T-like disorder (ATLD), Werner's syndrome, Bloom's syndrome, Rothmund-Thompson syndrome, Cockayne's syndrome (CS), Trichothiodystrophy, ATR-Seckel syndrome, LIG4 syndrome, Human immunodeficiency with microcephaly, Spinocerebellar ataxia with axonal neuropathy, Ataxia with oculomotor apraxia 1, Ataxia with oculomotor apraxia 2, Diamond Blackfan anemia, Rapadilino syndrome, Turcot Syndrome, Seckle Syndrome, Lynch syndrome, NBS-like syndrome, and RIDDLE Syndrome.

As used herein, the terms “effective amount,” “effective dose,” “sufficient amount,” “amount effective to,” “therapeutically effective amount,” or grammatical equivalents thereof mean a dosage sufficient to produce a desired result, to ameliorate, or in some manner, reduce a symptom or stop or reverse progression of a condition and provide either a subjective relief of a symptom(s) or an objectively identifiable improvement as noted by a clinician or other qualified observer. Amelioration of a symptom of a particular condition by administration of a pharmaceutical composition described herein refers to any lessening, whether permanent or temporary, lasting or transit that can be associated with the administration of the pharmaceutical composition. With respect to “effective amount,” “effective dose,” “sufficient amount,” “amount effective to,” or “therapeutically effective amount” of a pharmaceutical composition, the dosing range varies with the pharmaceutical composition used, the route of administration and the potency of the particular pharmaceutical composition.

The terms “individual,” “subject,” “host,” and “patient,” used interchangeably herein, refer to a mammal, including, but not limited to, murines, simians, felines, canines, equines, bovines, mammalian farm animals, mammalian sport animals, and mammalian pets and humans. Preferred is a human.

As used herein an “inflammatory disorder” is a condition or disease associated with inflammation, including but not limited to, septic shock, obesity-related inflammation, Parkinson's Disease, Crohn's Disease, Alzheimer's Disease, cardiovascular disease, inflammatory bowel disease, chronic obstructive pulmonary disease, an allergic reaction, an autoimmune disease, blood inflammation, joint inflammation, arthritis, asthma, ulcerative colitis, hepatitis (e.g., viral chronic hepatitis), psoriasis, atopic dermatitis, pemphigus, glomerulonephritis, atherosclerosis, sarcoidosis, rheumatoid arthritis, psoriatic arthritis, ankylosing spondylitis, Wegner's syndrome, Goodpasture's syndrome, giant cell arteritis, polyarteritis nodosa, idiopathic pulmonary fibrosis, acute lung injury, post-influenza pneumonia, SARS, tuberculosis, malaria, sepsis, cerebral malaria, Chagas disease, schistosomiasis, bacterial and viral meningitis, cystic fibrosis, multiple sclerosis, encephalomyelitis, sickle cell anemia, pancreatitis, transplantation (e.g., host-mediated rejection of transplanted tissue such as hematopoietic stem cells or an organ, graft mediated host response, such as graft vs. host disease), systemic lupus erythematosis, autoimmune diabetes, thyroiditis, radiation pneumonitis, respiratory inflammation and pulmonary inflammation.

As used herein, nicotinamide adenine dinucleotide or “NAD⁺” and its derivative compounds are known as essential coenzymes in cellular redox reactions in all living organisms. Several lines of evidence have also shown that NAD participates in a number of important signaling pathways in mammalian cells, including poly(ADP-ribosyl)ation in DNA repair (Menissier de Murcia et al. EMBO J. 22:2255-2263 (2003)), mono-ADP-ribosylation in the immune response and G protein-coupled signaling (Corda et al. EMBO J. 22:1953-8 (2003)), and the synthesis of cyclic ADP-ribose and nicotinate adenine dinucleotide phosphate (NAADP) in intracellular calcium signaling (Lee, Annu. Rev. Pharmacol. Toxicol., 41:317-345 (2001)). It has also been shown that NAD and its derivatives play an important role in transcriptional regulation (Lin et al. Curr. Opin. Cell. Biol. 15:241-246 (2003); Imai et al. Nature 403:795-800 (2000); Landry et al. Biochem. Biophys. Res. Commun. 278:685-690 (2000); Smith et al. Proc. Natl. Acad. Sci. USA 97:6658-6663 (2000)).

The NAD biosynthesis pathways have been characterized in prokaryotes by using Escherichia coli and Salmonella typhimurium (Penfound et al. Cellular and Molecular Biology, p. 721-730, ed. Neidhardt, F. C., 1996, ASM Press: Washington, D.C.) and in yeast (Lin et al. Curr. Opin. Cell. Biol. 15:241-246 (2003); Denu Trends Biochem. Sci., 28:41-48 (2003)). In prokaryotes and lower eukaryotes, NAD is synthesized by the de novo pathway via quinolinic acid and by the salvage pathway via nicotinic acid (Penfound, Id.). In yeast, the de novo pathway begins with tryptophan, which is converted to nicotinic acid mononucleotide (NaMN) through six enzymatic steps and one non-enzymatic reaction (Lin et al. Curr. Opin. Cell. Biol. 15:241-246 (2003)).

In mammals, NAD⁺ is generated from nicotinamide in a salvage pathway wherein nicotinamide phosphoribosyltransferase (NAMPT) converts nicotinamide to nicotinamide mononucleotide (NMN) which is then converted to NAD⁺ by nicotinamide mononucleotide adenylyltransferase (NMNAT) (Canto et al. Cold Spring Harbor symposia on quantitative biology 76, 291-298 (2011)).

As used herein, “PARP14” stands for Poly(ADP-ribose) polymerase 14. Alternative names include KIAA1268, B-aggressive lymphoma 2, B-aggressive lymphoma protein 2, collaborator of STATE, ARTD-8, or BAL2. Poly(ADP-ribosyl)ation is an immediate DNA damage-dependent posttranslational modification of histones and other nuclear proteins that contributes to the survival of injured proliferating cells. PARP14 belongs to the superfamily of enzymes that perform this modification (Ame et al., BioEssays 26: 882-893, 2004).

By sequencing clones obtained from a size-fractionated adult brain cDNA library, Nagase et al. DNA Res. 6: 337-345 (1999) cloned a partial PARP14 cDNA, which they designated KIAA1268. The cDNA contains repetitive elements in its 3′ UTR. RT-PCR ELISA detected intermediate to high expression in all tissues and specific brain regions examined. Highest levels were in lung, liver, kidney, spleen, and fetal liver.

Ame et al. (2004) reported that the full-length PARP14 protein contains 1,518 amino acids and has a calculated molecular mass of 170.6 kD. It contains two central domains similar to the C-terminal domain of the macroH2A histone protein (H2AFY; 610054) and a C-terminal region containing a WWE domain, which is found in proteins associated with ubiquitination, followed by a catalytic domain.

By database analysis using PARP9 (612065) as query, followed by 5′ and 3′ RACE of normal mature B cells and primary diffuse large B-cell lymphoma cells, Aguiar et al. J. Biol. Chem. 280: 33756-33765, 2005 (2005) cloned PARP14, which they called BAL2. They identified two alternatively spliced transcripts BAL2B, which contains a putative C-terminal PARP catalytic domain, and BAL2A, which lacks this domain. Both deduced PARP14 isoforms contain three macrodomains. Northern blot analysis detected high PARP14 expression in spleen, lymphocytes, and peripheral blood leukocytes.

By radiation hybrid analysis, Nagase et al. (1999) mapped the PARP14 gene to chromosome 3. Ame et al. (2004) reported that the PARP14 gene maps to chromosome 3q21.1. By genomic sequence analysis, Aguiar et al. (2005) showed that PARP14, PARP9, and PARP15 (612066) mapped in tandem within a 200-kb region on chromosome 3q21. Representative PARP14 sequences are set forth below.

The amino acid sequence information for the aforementioned proteins are well known in the art and readily available on publicly available databases, such as the National Center for Biotechnology Information (NCBI). For example, amino acid sequences derived from publicly available sequence databases are provided below in Table 1.

TABLE 1
PARP14 and homologs thereof. Bolded, italicized, highlighted, and
underlined amino acids reflect key amino acids in Macro Domain 1.
Deletion of, or mutation to, one or more amino acids, individually or
combined, may abolish PARP14 activity. Bolded and underlined amino
acids reflect key phosphorylation sites important for NADase activity.

NP_060024.2 Homo sapiens 24432106 (SEQ ID NO: 1) (1801 aa)

| | |
|---|---|
| 1 | MAVPGSFPLL VEGSWGPDPP KNLNTKLQMY FQSPKRSGGG ECEVRQDPRS PSRFLVFFYP |
| 61 | EDVRQKVLER KNHELVWQGK GTFKLTVQLP ATPDEIDHVF EEELLTKESK TKEDVKEPDV |
| 121 | SEELDTKLPL DGGLDKMEDI PEECENISSL VAFENLKANV TDIMLILLVE NISGLSNDDF |
| 181 | QVEIIRDFDV AVVTFQKHID TIRFVDDCTK HHSIKQLQLS PRLLEVTNTI RVENLPPGAD |
| 241 | DYSLKLFFEN PYNGGGRVAN VEYFPEESSA LIEFFDRKVL DTIMATKLDF NKMPLSVFPY |
| 301 | YASLGTALYG KEKPLIKLPA PFEESLDLPL WKFLQKKNHL IEEINDEMRR CHCELTWSQL |
| 361 | SGKVTIRPAA TLVNEGRPRI KTWQADTSTT LSSIRSKYKV NPIKVDPTMW DTIKNDVKDD |
| 421 | RILIEFDTLK EMVILAGKSE DVQSIEVQVR ELIESTTQKI KREEQSLKEK MIISPGRYFL |
| 481 | LCHSSLLDHL LTECPEIEIC YDRVTQHLCL KGPSADVYKA KCEIQEKVYT MAQKNIQVSP |
| 541 | EIFQFLQQVN WKEFSKCLFI AQKILALYEL EGTTVLLTSC SSEALLEAEK QMLSALNYKR |
| 601 | IEVENKEVLH GKKWKGLTHN LLKKQNSSPN TVIINELTSE TTAEVIITGC VKEVNETYKL |
| 661 | LFNFVEQNMK IERLVEVKPS LVIDYLKTEK KLFWPKIKKV NVQVSFNPEN KQKGILLTGS |
| 721 | KTEVLKAVDI VKQVWDSVCV KSVHTDKPGA KQFFQDKARF YQSEIKRLFG CYIELQENEV |
| 781 | MKEGGSPAGQ KCFSRTVLAP GVVLIVQQGD LARLPVDVVV NASNEDLKHY [symbol] AAALSKA |
| 841 | AGPELQADCD QIVKREGRLL PGNATISKAG KLPYHHVIHA VGPRWSGYEA PRCVYLLRRA |
| 901 | VQLSLCLAEK YKYRSIAIPA ISS [symbol] FPL GRCVETIVSA IKENFQFKKD GHCLKEIYLV |
| 961 | DVSEKTVEAF AEAVKTVFKA TLPDTAAPPG LPPAAAGPGK TSWEKGSLVS PGGLQMLLVK |
| 1021 | EGVQNAKTDV VVNSVPLDLV LSRGPLSKSL LEKAGPELQE ELDTVGQGVA VSMGTVLKTS |
| 1081 | SWNLDCRYVL HVVAPEWRNG STSSLKIMED IIRECMEITE SLSLKSIAFP AIGTGNLGFP |
| 1141 | KNIFAELIIS EVFKFSSKNQ LKTLQEVHFL LHPSDHENIQ AFSDEFARRA NGNLVSDKIP |
| 1201 | KAKDTQGFYG TVSSPDSGVY EMKIGSIIFQ VASGDITKEE ADVIVNSTSN SFNLKAGVSK |
| 1261 | AILECAGQNV ERECSQQAQQ RKNDYIITGG GFLRCKNIIH VIGGNDVKSS VSSVLQECEK |
| 1321 | KNYSSICLPA IGTGNAKQHP DKVAEAIIDA IEDFVQKGSA QSVKKVKVVI FLPQVLDVFY |
| 1381 | ANMKKREGTQ LSSQQSVMSK LASFLGFSKQ SPQKKNHLVL EKKTESATFR VCGENVTCVE |
| 1441 | YAISWLQDLI EKEQCPYTSE DECIKDFDEK EYQELNELQK KLNINISLDH KRPLIKVLGI |
| 1501 | SRDVMQARDE IEAMIKRVRL AKEQESRADC ISEFIEWQYN DNNTSHCFNK MTNLKLEDAR |
| 1561 | REKKKTVDVK INHRHYTVNL NTYTATDTKG HSLSVQRLTK SKVDIPAHWS DMKQQNFCVV |
| 1621 | ELLPSDPEYN TVASKFNQTC SHFRIEKIER IQNPDLWNSY QAKKKTMDAK NGQTMNEKQL |
| 1681 | FHGTDAGSVP HVNRNGFNRS YAGKNAVAYG KGTYFAVNAN YSANDTYSRP DANGRKHVYY |
| 1741 | VRVLTGIYTH GNHSLIVPPS KNPQNPTDLY DTVTDNVHHP SLFVAFYDYQ AYPEYLITFR |
| 1801 | K |

Additional human sequences may include, but not limited to, NCBI
Genbank Reference numbers: XP_011511230.1 and XP_011511231.1. Serine
1403 is a key phosphorylation site likely important for PARP14 NADase
activity.
XP_516695.3 P. troglodytes 334312652 (SEQ ID NO: 2) (1801 aa)

| | |
|---|---|
| 1 | MAVPGSFPLL VEGSWGPDPP KNLNTKLQMY FQSPKRSGGG ECEVRQDPRS PSRFLVFFYP |
| 61 | EDVRQKVLER KNHELVWQGK GTFKLTVQLP ATPDEIHHVF EEELLTKESK TKEDVKEPDV |
| 121 | SEELDTKLPL DGGLDKMEDI PEECENISSL VAFENLKANV TDIMLILLVE NISGLSNDDF |
| 181 | QVEIIRDFDV AVVTFQKHID TIRFVDDCTK HHSIKQLQLS PRLLEVTNTI RVENLPPGAD |
| 241 | DYSLKLFFEN PYNGGGRVAN VEYFPEESSA LIEFFDRKVL DTIMATKLDF NKMPLSVFPY |
| 301 | YASLGTALYG KEKPLIKLPA PFEESLDLPL WKFLQKKNHL IEEINDEMRR CHCELTWSQL |
| 361 | SGKVTIRPAA TLVNEGRPRI KTWQADTSTT LSSIRSKYKV IPIKVDPTMW DTIKNDVKDD |
| 421 | RILIEFDTLK EMVILAGKSE DVQSIEVQVK ELIESTTQKI KREEQSLKEK MIISPGKYFL |
| 481 | LCHSSLLDHL LTECPEIEIC YYRVTQHLCL KGPSADVYKA KCEIQEKVYT MAQKNIQVSP |
| 541 | EIFQFLQQVN WKEFSKCLFI AQKILALYEL EGTTVLLTSC SSEALLEAEK QMLSALSYKR |
| 601 | IEVENKEVLH GKKWKGLTHN LLKKQNSSPN TVIINELISE TTAEVIITGC VKEVNETYKL |
| 661 | LFNFIEQNMK IERLVEVKPS LVIDYLKTEK KLFWPKINKV NVQVSFNPEN KQKGILLTGS |
| 721 | KTEVLKAVDI VKQVWDSVCV KSVHIDKPGA KQFFQDKARF YQSEIKRLFG CYIELQENEV |
| 781 | MKEGGSPAGQ KCFSRTVLAP GVVLIVQQGD LAQLPVDVVV NASNEDLKHY [symbol] AAALSKA |
| 841 | AGPELQADCD QIVKREGRLL PGNATISKAG KLPYHHVIHA VGPRWSGYEA PRCVYLLRRA |
| 901 | VQRSLCLAEK YKYRSIAIPA ISS [symbol] FPL GRCVETIVSA IKENFQFKKD GHCLKEIYLV |
| 961 | DVSEKTVEAF AEAVKTVFKA TLPDTAAPPG LPPAAAGPGK TSWEKGSLVS PGGLQMLLVK |
| 1021 | EDVQNAKTDV VVNSVPLDLV LSRGPLSKSL LEKAGPELQE ELDTVGQGVA VSMGTVLKTS |
| 1081 | SWNLDCRYVL HVVAPEWRNG STSSLKIMED IIRECMEITE SLSLKSIAFP AIGTGNLGFP |
| 1141 | KNIFAELIIS EVFKFSSKNQ LKTLQEVHFL LHPSDHENIQ AFSDEFARRA NGNLVSDKIP |
| 1201 | KAKDTQGFYG TVSSPDSGVY EMKIGSIIFQ VASGDITKEE ADVIVNSTSN SFNLKAGVSK |
| 1261 | AILECAGQNV EMECSQQAQQ RKNDYIITGG GFLRCKNIIH VIGGNDVKSS VSSVLQECEK |
| 1321 | KNYSSICLPA IGTGNAKQYP DKVAEAIIDA IEDFVQKGSA QSVKKVKVVI FLPEVLDVFY |
| 1381 | ANMKKREGTQ LSSQQSVMSK LASFLGFSKQ SPQKKNHLVL EKKTESATFR VCGENVTCVE |
| 1441 | YAISWLQDLI EKEQCPYTSE DECIKDFDEK EYQELNELQK KLNINISLDH KRPLIKVLGI |
| 1501 | SRDVMQARDE IEAMIKRVRL AKEQESRADC ISEFIEWQYN DNNTSHCFDK MTNLKLEDAR |
| 1561 | REKKKTIDVK INHRHYTVNL NTYTATDAKG HSLSVQRLTK SKVDIPAHWS DMKQQNFCVV |
| 1621 | KLLPSDPEYN TVASKFNQTC SHFRIEKIER IQNPDLWNSY QAKKKTMDAK NGQTMNEKQL |
| 1681 | FHGTDAGSVP HVNRNGFNRS YAGKNAVAYG KGTYFAVNAN YSASDTYSRP DANGRKHVYY |
| 1741 | VRVLTGIYTH GNHSLIVPPS KNPQNPTDLY DTVTDNVHHP SLEVAFYDYQ AYPEYLITFR |
| 1801 | K |

Additional chimpanzee sequences may include, but not limited to, NCBI
Genbank Reference numbers: XP_009444569.1 and XP_016797243.1.
XP_001105869.2 M. mulatta (SEQ ID NO: 3) (1800 aa)

| | |
|---|---|
| 1 | MAVPGSFPLL VEGSWGPDPP KNLSTKLQMY FQSRKKSGGG ECEVHQDPRS PSRFLVLFYP |
| 61 | EDVRQKVLER KNHELVLQGK GTFKLTVQLP ETTDEIHHVF EEELLTQESK TKEDVKEPDV |
| 121 | SEELEAKLPL DDGSDNIEDI PEECENISSL VAFENLKANV TDIILTLLVE NISGLSSDDF |
| 181 | QLEVIRDFDV AVVTFQKHID TVRFVDDCTR HHSIKQLQLS PRLLEVTKTI RVENLPPGVD |
| 241 | DYSLKLFFEN PYSGGGRVAN VEYFPEESSA LIEFFDRKVL DTIMATKLDF NKMPLSVFPY |
| 301 | YASLGTALYG KEKPLIKLPA PFEESLDLPL WKFLQKKNHL IEEINDEMRR CHCELTWSQL |
| 361 | SGKVTIRPAA TLVNEGRPRI KTWQADTSTA LSSIRSKYKV TPIKVDPTMW DTIKNDVKDD |
| 421 | RILIEFDTLK EMIILAGKSE DVQSIEVHVK ELIESTTQKI KREEQSLKEK MVISPGRYFL |
| 481 | LCHSSLLDHL HTECPEIEIC YDGVTQHLCL KGPSVDVYKV KCAIQEKVYT MAQKNIQVSP |
| 541 | EIFQFLQQVN WKEFSKCLFI AQEILALYEL EGTTVLLTSC SSEALLEAEK QMLSALDYKR |
| 601 | IEVENKEVLN GKNWKGLTHN LRKTKNFSPN TVIINELSSE TTAEVIITGC VKKVNETYAL |
| 661 | LFNFIEQNMK IERLVEVKPS LVIDYLKTEK KLFLPKIKRT NVQVSFNPEN KQKGILLTGS |
| 721 | KTEVLKAMDI VKQARDSVCV KSVHIDKPGA KMFFRDKAWF YQREIKRLFG CYIELQENEE |
| 781 | VKEGGSPSGQ KCFSRTVLAP GVVLIVQQGD LAQLPVDVVV NASNEDLKHY [symbol] AAALSKA |
| 841 | AGPELQADCD QIVKREGRLL PGNATISKAG KLPYHHVIHA VGPRWNGCEA PRCVYLLRRA |
| 901 | VQLSLCLAEK YKYRSIAIPA ISS [symbol] FPL GRCVETIVSA IKENFQRKKD GHYLKEIYLV |
| 961 | DVSEKTVEAF AEAVKTVFKA TLPDTAAPPS LPAAAGPGKT SWKQGSLVSP GGLQMLLVKE |
| 1021 | GVQNAKTDVV VNSVPSDLEL SRGPLSKALL EKAGPALQEE LDTVGQGMTV SVGTVLKTNS |
| 1081 | WNLDCRYVLH VVAPEWRNGS TSSLKVMEDI IRECMEITDS LSLKSIAFPA IGTGNLGFPK |
| 1141 | TIFAELIISE VEKESSKNQL KTLQEVHFLL HPSDHENIKA FSDEFARRAN GNLISDKISK |
| 1201 | AEDTQGFYGT VSSPDLGVHE MKIGSIIFQV ASGDITKEEA DVIVNSTSKS FNLKAGVSKA |
| 1261 | ILECAGQNVE MECSQQAQQH KNDYIITGGG FLKCKNIIHV IGGNDVKSSV SSVLQECEKQ |
| 1321 | NYSSICLPAI GTGNAKQDPE KVAEAIIDAI EDFVQKGSAK SVKKVKVVIF LPQVLDVFYA |
| 1381 | IMKKREGTQV SSQQSMMSKL ASFLRISKQP PKKENHLVLE KKTESATFRV CGENVMCVEQ |
| 1441 | TISWLQDLIE KEQCPYTSED EYIKYFDEKE YQKLKELQEK LNINIFLDNK RPLIKVLGIS |
| 1501 | RDVMQARDEI EAMIKRVRLA KEQESRADCI SEFIEWQYND NNTFYRFDKI TNLKLEDARR |
| 1561 | EKKKTIDVKI NHQHYTVNLN TYTATDAKGL SLSVQRLVKS EVDIPAHWSD MKQQNFCVVE |
| 1621 | LQPRDPEYNT VASKFNQTCA HFRIEKIERI QNPDLWNSYQ AKKKTMDAKN GQTMNEKQLF |
| 1681 | HGTDAGSVPY VNRNGFNRSY AGKNAVAYGK GTYFAVNANY SANDTYSRPD ANGRKHMYYV |
| 1741 | RVLTGIYTHG NQSLIVPPSK NPQNPTDLYD TVTDNVQHPN LFVVFYDYQA YPEYLITFRR |

XP_850880.2 C. lupus (SEQ ID NO: 4) (1792 aa)

| | |
|---|---|
| 1 | MAAPGSFPLL VEGSWGPDPP ESLPTKLQLY FQSARRSGGG ACEFLREPGS PPRELVVEHL |
| 61 | EDVQQRVLER KNHELVWLGK GTFQLTVQLP TAPDEVQDVF EGEIPTKELE TKEHVKEPDV |
| 121 | SEELDIKLSL SRRGERMEDT SEECENTSSL VAFENLKANV TDIMLILLVE NVSGLSSNDF |
| 181 | KVEVLRDFDV AVVTFQTHTD AVKFVDDCAR HHSVKQLQLS PRLLEVTKTI RVENLPPGVN |
| 241 | DYNLKCLFEN PQNGGGRVAS IECFPEESSA LIEFFDKKVL HTIVTKKLEF KHMPLSVFPY |
| 301 | YTSLGTALYG KEKPLIKLPA PFREPLDLPL WRFLQQKNHL IKEINDEARR CHCELIWSKL |
| 361 | SAEVTIRPAT TLFCLGRLKI RTWREDVSMA FSSIRSKYKV TTLTVDPTVW DTVRNHLEDD |
| 421 | RILIEFDTLT GTVTLVGKSE DVQNMEPQIK ELIESTVQKI KREEQSLEEK VALSPGRYSL |
| 481 | LCHSNALEHL RTECPEVEMC FDKASQHMCF KGFHADVNKA KCEIQKKMDI MVQKSIELPP |
| 541 | EIFQFLQQVD CVEFSKSLLV AQKILAVYEL AGTAVVLTGY SSAVLSDAAK QMVTALSYKR |
| 601 | LDLKDGEVLK GRKWKGLTHS LHRRHNSPSR TVIIDELTSE TKAEVVIAGC TSQVNETYSL |
| 661 | LFSFVEKHMR VERLIEIEPP IVIDYLRREK KLCWQKMKRT NVQVIFNPEN RAQGVLLIGP |
| 721 | QARVLEGVSL LGQVREAVRV ESLQVHTPGA SRLFREKAPY YRSEVQRRFG CCLELQEAAG |
| 781 | AAGRGDAHRG LPRVELRPGV ALAVQRGDLT RFPAAQAVVS PASGDLQLR [symbol] [symbol] AALAQAA |
| 841 | GPALQEDCSR LLRTAGPVPV GGAVASTAGK LPYRLVIHAV GPQWKDSEAS RCVSQLKAAV |
| 901 | KQSLRLAEAH GCRSVAIPAI SS [symbol] FPLA VCVRTIVLAV QESCQLSHDG HALKEIYLVD |
| 961 | TAEKTVQAFA DTVTALAEVP GPAAVQPGPT GVHASGQVSL SQGNLRVLLV KGDAQRAAAD |
| 1021 | VIVNSVPVNL KLNRGLLSQA LLEKAGPKLQ EELDMVGRAV AVGMGTVLQT SGCDLHCRYV |
| 1081 | LHVVAPDWEE GSTSSQKIMR DIIRKCLEIT ESLSLRSIAF PAIGTGNLGF PKTIFAELIT |
| 1141 | SEVLTFSSKT QLAALQEVQF LLHPNDHENI QAFSHVFARR TNGNVVSDTI PKAEDAQGSY |
| 1201 | GTVSSPNVDM HEMKIGPIIF QVASGDITKE EADVIVNSTS KTFNLKAGVS KAILKCAGQN |
| 1261 | VEMACSLLAQ TGNSDYIVTE GGLLKCKNII HVIGGNDVKK SISFVLQECE KRNYSSICLP |
| 1321 | AIGTGNAQKD PDKVADAIID AIEDFIQKGM LKSVKKVKVV IFLPHLLDVF YDSMKKREAS |
| 1381 | QASPQQSLES KLKSSVSSPS QSPQKQNPLV LKKKTESATF QVCGENVKCM KNALSWIQDL |
| 1441 | ITKELCPYTN EDECIKDFNE KEYHKLNELQ ENLNIAICLD SERPLIEVFG TGNDLTQARN |
| 15D1 | AIEEMIKGIR LAKEQKSQAD FISEFIEWQY YNNGTFHSFD KITNLQLENA RKAKKRTTLV |
| 1561 | KVNHKSYTVD LVTNIATDTK GHHLPVKRFM KSEVVIPEHW SDMKQQDVCV VELQPGHAEY |
| 1621 | DTVATKFNQT CSHLYIEKIE RIQNPHLWNS YQTKKKAMDA KNGHKNNEKQ LFHGTDADSV |
| 1681 | PHVNHNGFNR SYAGKNAVAY GKGTYFAVNA RYSANDTYSR PDINGKKHMY YVRVLTGTYT |
| 1741 | RGNQSLIVPP PKSADNPTDL YDTVTDCVQN PGLFVVFYDY QAYPEYLITF TY |

Additional dog sequences may include, but not limited to, NCBI
Genbank Reference numbers: XP_005639627.1; XP_022269481.1;
XP_022269482.1; and XP_0222694831
NP_001193467.1 B. taurus (SEQ ID NO: 5) (1797 aa)

| | |
|---|---|
| 1 | MAAPGSFPLL VEGSWGSQPP KNLSTKLQMY FQSRKRSGGG ECEVCPVPGS SNRFLVLFYP |
| 61 | DDVRQQVLER ESHELEWPGK GTFKLTVQIP TVPDEVQERK IPTKESKTKE QVEESDASKE |
| 121 | LEEDLSLSRR SEKEEDTPKE CEDISSLVAF ENLKANVTDE MLIFLVENIS GLTNDNFKLE |
| 181 | IIRDFNVAVV TFQTYTDALK FVGECPKHNS VKRLQLSARL LEVTKTIRVE NLPPGVHDHD |
| 241 | LKCLFENPHN GGGRVASIEY FPEESSALIE FFDRKVMDTI MTKKLDLNNM PLSVFPYYPS |
| 301 | LGTALYGEEK PLIKLPASFR EPLDLSLWKF LQKKKHLTEE IRDKMRYCHC ELTWCQLSNE |
| 361 | VIIRPAATLV NQGRLRVKTW RKDASTLFSD IRSEYTVTSF KVDPTVWDTI KDSLEDDRIL |
| 421 | IEFDALASII TLVGESEDVQ NIEPQIKDLI ESTIQNIKRE EQSLKQKLAI NSSKYSLLLH |
| 481 | SGIQEQLHTE CPDVEIYYDE ASQHMCFKGL PAEVYKVKCE IQEKVYTMAQ RNIHVHPEVF |
| 541 | QFLQQVDCAE FSKSLFTAQN ILAVYELEGT TVLLTGFSSE VLSEAEKQMQ STLICNCIDI |
| 601 | EDREVLNGKN WKGLTGNLCK KHNSSSKTVV IQEVTSGTAA QVIIAGCERE VRKIYGLLSD |
| 661 | FVEKHSKIER WTGVKSSLIV DYLKAEKQIW SNIRGMNVQV NFNPENKQKS ISLIGPKAKV |
| 721 | LEGMKIVRQA LDSVCVKSVH TDKPGAGQFF QDKARYYKSE VRRLFGCFIE LKKDEEKEGR |
| 781 | GTGGQRCFFR TDLAPGVSLI VQQGDLTHFP VQVVVNAANE ELKLR [symbol] AA ALLRAAGPEL |
| 841 | QEDCDQILKK RGKLSPPYAV ISKAGKLPYQ HVIHAVGPQW KSDEAQKCVL QLKNAVKESL |
| 901 | NLAVKHKYQS IAIPAISS [symbol] FPLSQCVE AIVLAIKEWF QCKKDGCTLK EIYLVDIAEK |
| 961 | TVEAFAESAK SVFKDTLPDI ASLPSLPAAA QPDLRKDHGN GSSLLTPEGL RIHLVKEDVQ |
| 1021 | NATTHVVVNS ISSDLELDRG PLSKAFLEKA GQKLQEELKT AGQGVVVDVG TVLRTSGCNL |
| 1081 | HCQHVLHVVA PNWRNDRPSS QKIMENIIRE CLEITESLSL NSIAFPAIGT GNLGFPKNVF |
| 1141 | AELIISEVFT FSRKNQPRTL QEVWFLLHPS DHETIQAFSD EFARRANGNF ISDKVSKAES |
| 1201 | TQDFYGTISN PDLGVYEMKI GPIIFQVASG DISKEEADVI VNSTSKSFNL KAGVSKAILG |
| 1261 | RAGRNVEIEC SRQAQQGHSD YIITQGGDLK CKNIIHVIGG NDVKRSVTCV LQECEKRHYS |
| 1321 | SVCLPAIGTG NAKQDPDKVA VAILDAIEEF IQKRLVQSMK KVKVVIFQPQ VLDVFRANMT |
| 1381 | KREGYQASFQ QSVISKIASF LGFSSKSPKK QTLVLEKKTE LAVFQVCGRN VKNVENALLW |
| 1441 | IQDLIQKEQC PYSNEDECIK NFDVNEYKEL NELQKKLNIS ISLNRERPLI EVSGIIKDVI |
| 1501 | QARNAIEDMI KRVRLSKEQE SLADRTSDFV EWQYEDYNNI FHSFDKITNM QLEDAKKQKR |
| 1561 | KTIDVKINNQ SYTVDLKTYI ATDAKGNSLS VQRHTKSEVE LPPYWSDMKQ QKVCVVELQP |
| 1621 | DHPEYRTVAS KFQETCAHFK IERIERIQNP ELWKHYQTKK NSMDAKNGQV TNEKLLFHGT |
| 1681 | DADSVALVNG KGFNRSYAGK NATAYGKGTY FAVNASYSAS DVYSRPDING KKHMYYVRVL |
| 1741 | TGCYTLGNGS LIVPPPKDHQ NPTDSYDTVT DCLQNPNLFV VFYDYQAYPE YLITFRY |

NP_001034619.2 1 M. musculus (SEQ ID NO: 6) (1817 aa)

| | |
|---|---|
| 1 | MAASGSFPLL VEGSWGPDPP KNLINKLQVY FQSRKKSGGG ECEVVPEPGN PARFRVLFSP |
| 61 | EDVRQNVLER GNHELVWQEK GTFKLTVLMP TDPEEASASK KSRKESPEEE SKTKEDAVKQ |
| 121 | GDLDITHSPS SGSEKTEDVP KECENISSMV AFENLPEKVS EMVLTILVEN ISGLPSDDFK |
| 181 | VEVNRDFAVA VVTFQKPIDI KKFIVDCISH RSNQQLQLAP RLLETTNVVR VENLPPGVDE |
| 241 | YQLQLFFENP FNGGGRVARV ECFPEESSAL VEFCDSKVLD TVMAKTHSYN KMPLSVFPYY |
| 301 | PSLGTALYGE EKPLIKLPAS FQESLDLPLW KFFQKNNHLI EEINNEMRCC HCELTWSEIN |
| 361 | GKLTIRPAAT LVNHRLSIKT WQRDASAVLS GIKSKYGVEL FEVCSPVWDI IKHELESGDD |
| 421 | RVLVEFEKES LNIAGKSEDV QGMSQKIREL IESTTEKLRR EEQSLKEKVA ISPGKHYLLH |
| 481 | HSGFLKDLSK GFPEMEISYD ATAQFLYLKG FRADVYKVKC DIQEKVFSMA QKDVQVSSEV |
| 541 | FEFLQQVDSQ RLSKSLFEAQ NILAIYELKG TALFLVGSSF KDLAEAETKM LSALSHKQIE |
| 601 | VEDKEVLISN GWKKKVHPLQ KRHSSCATII VQNELTSETP AKVIVTGCVK EVNEIHRQLF |
| 661 | EYLENNMKVE RALKIKPSLI VDYLRTDKRL LSKIKKAHVY VHFKPKDNPN SILLTGCKSK |
| 721 | VLECMNLVKE IQDSVCVQRF QTDKAGVRHF FKDKESYYKT EIGRQFGCVI ELEEDREEKG |
| 781 | EEEDGEEEEG EEEGESSINE QKCHLQRDIA PGVKLFVLEE DLSRFPVDVV VNAANENLKH |
| 841 | I [symbol] AQALSK AAGPELQTEC DQIVKEGGVV LPGNAVISKA GKLPCHHVIH AVGPRWKGDK |
| 901 | VLECVSLLKK VVRQSLSLAE EHRCRSIAMP AVSA [symbol] FP LELCVANIVS AIKEHFQHKR |
| 961 | DTHTLKKIYL VGLPAKVARA FAEAVKTTYK DSLSHTAFPS SLKALVPLGK TPQKQGSLLV |
| 1021 | SPEGLRIRLV EEGVQNATTH AIVNSISPDL KLNKGPLSQA FLEKAGPKLQ EELTRSGQGV |
| 1081 | SVDVGTILQT SGCNLNSRHV FHVVPPPWKS NNSAWSLKIM KNIIRDCLKT TENLSLQSIA |
| 1141 | FPAIGTGNLR FPKPEFAKLI ISEVLKFSSR NQLKTLQEVQ FLLHPKDHEN IQAFSDEFDK |
| 1201 | RNNGDPSDKN PKAEDTQGIY GSLSSPTLGM HEMNIGPILF QVATGNIIKE VADVIVNSTT |
| 1261 | LTFDLKSGVS KAILEGAGQN VEQECSLLAK QSNHGYIVTG GGLLQCKNII HVVGGNDVKK |
| 1321 | SVSCVLEECE QRNYSSICLP AIGTGNAQQD PNVVAKAIID AIEEFVQKKS VQAVKRVKVV |
| 1381 | IFQPHILQFF YDNMKEREGS PAPPKQSPAK QSVMSKIASF LGFPKQASPK KNTLVLEKKI |
| 1441 | EHTVFQVCGS GVDSVNKTIS WLKELITKEQ LSYTNDDECV SDFDMEEYEK LNEIQKELNI |
| 15D1 | TIEMNQKKTS IQVSGISRDV IKARDEIEGM IKSIRLAKEK ESQADYISTY VEWQYIDKNI |
| 1561 | TQCFDKMTNM KLEVAWKAKK KDTVVQIHNQ DFTVDLSTNT ATAPQGQTFT VQRLVKAEAE |
| 1621 | IPANWSDMKQ DKLLLVNLQT SDPEYNMVAS AFRQTCSNFF IEKIERIQNP ALWRRYQAYK |
| 1681 | KSMDEKNGNV RNEKHLFHGT EASSLPQLNS NGFNRSYAGK NATAYGKGTY FAVKASYSAC |
| 1741 | DTYSRPDTNG RKYMYYVRVL TGNYTNGNAS LIVPPSRDPQ NAADLYDTVT DNDKNPSIFV |
| 1801 | VFYDNQTYPE YLITFRQ |

Additional mouse sequences may include, but not limited to, NCBI
Genbank Reference numbers: XP_006522455.1; XP_006522456.1; and
XP_006522457.2. Serine 1419 is a key phosphorylation site likely
important for PARP14 NADase activity.
NP_001178588. R. norvegicus (SEQ ID NO: 7) (1787 aa)

| | |
|---|---|
| 1 | MAASGPFPLL VEGSWGPDPP KNLINKLQMY FQSRKKSGGG ECEVVPEPGN PARFLVLFSP |
| 61 | EDVRQNVLEK ENHELVWQGK GTFKMTVQLP ADPGQASVSK KAIPEEESKT KEDAVKPGDL |
| 121 | DTTPSPSSRS EEMEDVPKEC ENISSMVAFE QLPEKVSEMV LTFLVENISG LPSDDFKVEL |
| 181 | IRDFAVAVVT FQKPIDTKKF IIDCISHRSN QQLQLVPRLL ETTNVVRVEN LPPGVDDYQL |
| 241 | QLFFENPFNG GGRVARVECF PEESSALVEF CDSKVLDTIM AKKHSFNKMP LSVFPYYPSL |
| 301 | GTALYGEEKP LVKLPASFQE SLDLPLWKFF QKNDHLMEEI NDEMRHCHCE LTWSEINGKL |
| 361 | TVRPAATLVS HRPSIKTWQR DASAALSGIR SKYDVMVFEV CSPVWDIIKH ELGDDRVLIE |
| 421 | FEKESLNVAG KSEDVQGISQ QIRELIERTT EKLRREEQSL EEKVAISSGK HFLLYHSGFL |
| 481 | KDLSKEYPEM EISYDATAQY LCIKGLRADV YKVKCEIQEK VFSMAQKNVP VSSEVFRFLQ |
| 541 | QVDGQRLSNA LFKAQNILAT YELKDNALFL IGSSPKDLAE AEAKMLSALS HKHIEVEDKE |
| 601 | VLRSNVWKKK IHPLQKRHGS YAAIMVKNEL TSGVPAEVVI AGCVKEVNEI HRQLFEFLEN |
| 661 | HMKVERVVKI DPPLIIDYLK TDKRLLSKTK KANVSVSFKT KDDPNSILLL GSKSKVLECL |
| 721 | DTVKEIRDSV CVRKFQTDKA GARHFFQDKE FYYKMEIRRL YGCIVELKEG EEGEEDSLKE |
| 781 | QKCYLQRDIG PGVTLIVHEG DLSLFPVDVV VNAANESLKH I [symbol] AGTLSK AAGPSLQAEC |
| 841 | DLIVKKGGMV PPGNAVISKA GKLPCRYVIH AVGPRWKGDK VLECVNLLKK AVKQSLTLAE |
| 901 | EYKCQSIAIP AISS [symbol] FP LDLCVASIAS AIKDNVQQKQ NTHSVKKIYL VDVSAKVAKA |
| 961 | FAEAVKTTYR DILSPASVKA LVPPGKMPQK QGKLLVSPEG LNIRLIEEGV QNAEAHAIVN |
| 1021 | SVSSDLILNK GPLSQAFLES AGPELQEELT KAGQGVSVSV GTVLQTSGCN LNSRSIFHVV |
| 1081 | TPPWKSNNSE WSLKIMKNII RDCLRTTEDL SLQSIAFPAI GTGNLGFPKP EFAKLIISEV |
| 1141 | LKFSSRNQLK TLQEVQFLLH PKDHENIQAF LDEFDKRSNG DPSSKNPKAE DTQGIYGSLS |
| 1201 | SPTLGVHEMN IGPILFQVAT GDIIKEVADV IVNSTSCTFN LKSGVSKAIL EGAGQNVEQE |
| 1261 | CSRLAQQGKE YIVTGGGLLK CKSIIHVDGG NDVKRSVSCI LEECEQRSFS SICLPAIGTG |
| 1321 | SAQQDPKVVA KAVLDAIEEF VQKKSVQAVK RVKVVIFQPH ILQFFYDNMK EREGSPAPVK |
| 1381 | PSVMSKIASF LGFPTQASPP KNTLVLEKKI ELTVFQICGP GVDSVEGTIS WLKSLITKEQ |
| 1441 | FSFTNEDECV RDFDTEEYRK LNEMQKRLNI IIELNQKKPL IKVSGISRDV VEARDEIDNM |
| 1501 | VKSIRLAKEK ENQADYVSTF VEWQYIANNT TRCFDKIANM QLEDAWKAKK KHTVVKIQNQ |
| 1561 | DFTVDLSTNT ATAPQGLSFT VQRLLKAEAE IPANWSDMKQ NKLLVVSLQT SDAEYTMVSR |
| 1621 | AFHQTCPNFV IEKIERIQNP ALWRRYQAYK KIMDEKNGNV INEKQLFHGT EFGSLAQLNS |
| 1681 | NGFNRSYAGK NATAYGKGTY FAVNASYSAH DTYSKPDANG KKYMYYVRVL TGNYTQGNAS |
| 1741 | LIVPPSRDPQ NATDLFDTVA DNVINPSIFV VFYDNQAYPE YLITFRK |

XP_422113.4 G. gallus (SEQ ID NO: 8) (3147 aa)

| | |
|---|---|
| 1 | METAPEPCQV ENPEVSLPSP LMVLENVREN VSSYMLTMLV ENISDLSEED GNFSVEMIPE |
| 61 | LQAAVVTFTG NAATADIVKK LNQSERAKKQ NLTAHLLELT NCIRAENIPP NTPSDYISIY |
| 121 | FENKKNGGAQ VANVFQQHEE GAAIIKFKDH RDVNNVLSKQ HSLNGTPISV YPYYASLGTA |
| 181 | LYGKEGPQIK QPDPVTVPLD PYIWRFLQRK NSLIEAIRSD MANCNCEITW PRGNCAEPKV |
| 241 | ILHPSAALSE RKRSVAQLVR TWNTVASTTF SYSISKYRVE KLKVSAEVWK AIRTSINHSE |
| 301 | LLMIPFTSKD LLLVVGHKDI LKTVVRELNL LIEKVTREIE REKQTTEEIV MLSPGDFAIL |
| 361 | QSIGLEERIR LEFPALQITY DHFQQIINLY GLREEVYKVK GEILVNMQKM TKKSVTLHPF |
| 421 | VFQFLQRIDN ETLSQSLFLS KHISAFYELG PDAVLLKGND DENLLKAEEE LKKELDYKSI |
| 481 | ALEDESILQK EEWTALTEEN CCNAAVTVIQ KEGEIIVAGF SQAVAKAFEE LFNFVDENTQ |
| 541 | VQKVIGGKPT VVLMYIKEEK TSVLDDLKNK GVKIDFNKVI SLRGPRREVL KGTTLIEQML |
| 601 | SGLHYKRVVI NEPGARSYLK ERVRFFADSV KNEFNCLIRL EEQPEEYPEE QREHSNVGKC |
| 661 | YTQMTVHGVL IAVYKADLCT HHVDVVVNAS NEDLKHI [symbol] AWALLQAAGP ELQAECDGVV |
| 721 | RMSGSLQAGD AVITGAGKLP CKQVIHAVGP RWKEQDAEKC VYLLKKTIKK SLQLAETYNH |
| 781 | RSIAFPSVSG [symbol] FPLHKC VNAIVSAIKK TLEEFKRDSS LKEIHLVDIT EDNVQAFIKA |
| 841 | LKEVFSDDVP LNNPVHQTSI VHRPTKRIAS RSNMNFALVT TEEGLNIMLK KGNIEDASTD |
| 901 | GVVISVGGDL QLEKGQLAKA LLSKAGPRLQ SDLNDEGLGK SPVEGSVFTT RGYNLSCCYV |
| 961 | FHAVTPGWSQ GSESAVKILG KIVTKCLQTA EELSLKSITF PAIGTGILGF PSSVVAKSLF |
| 1021 | DKVYEFSSKK KTNSLREVHF LLHPKDVNNI QAFSNEFERR CGNDVDEASK GTAFFGPISN |
| 1081 | PARDVYEMRI GSITFQVAAG DITKETGDVI VNISNQAFNL KTGVSKAILE GAGKEVENEC |
| 1141 | AELALQPNDG YITTEAGSLP CKKIIHFVAR DDIKVPVSKV LQECELQQYT SVTFPAIGTG |
| 1201 | QAGRFPDLVA DEMMDAITDF ARSNSTPSVK TIKIVIFQPH LLNVFHTSMK KRENDGKTKS |
| 1261 | KSLISRVTSF LIPEKHPPKE KRKAVLESKV DQAVVQICGE SEKEVEKAET WLKSTILKEQ |
| 1321 | FQKEINDDAI LDFDEAENEE LCDLQRRLNI ALFLGDNFIR ITGFNNDVWF AFSSIQQMIL |
| 1381 | RVKTAKYEAI KAELYQNLVE WKYLEKDSYV SFDSMTNMCL ESAFVRKQKD VSVIINKKKY |
| 1441 | KVDTEGRYAT NVEGHRITLI RIDKSADLES TTLPEKWDDM QNQQLKIVEL KPDTKDYRQV |
| 1501 | KERFLNTSPS LNLKIEKIER VQNPSLWKAY QIKKCQMDDK NGNNSNEKFL FHGTSAESLT |
| 1561 | FINNHGFNRS YAGMHAAQYG NGTYFAVNAS YSANDTYSKP DANGKKHMYL ARVLVGQYSQ |
| 1621 | GTQGAITPAA KNVGNTVDLF DSSTDNVNNP SMFIIFNDIQ AYPEYLITFT RINQEMANCH |
| 1681 | CYLKWPQTDC SQPEITLCPS SSISEQKGTM IKLIKTWKQD VSTEFSRIML RYISIKCKVS |
| 1741 | SAKWKDVKNR LMKNADLIIT DISEEMVVIA GNRAAVDSAE KEVRECMEKA MEESKREKQN |
| 1801 | IEIPVSITPG RYAVLRNAGL EENIHKEYPH LKISYDDTKE TVQLCGLSAE VYKVKANLLE |
| 1861 | KVLNMPSTSV DVNHNVLLYL RCANSKTMSE KLFAAKKINA FYELEDDTVI LFGETPKDLS |
| 1921 | EGEKQLKASL ACKSIDVKDC EVTKMKEWSH LLDALRKKYP SSQKTVVIQE PIGYGNTVTI |
| 1981 | TGFTETVEEV CQNLYDFIDR NTWVERKIPA KLVAAVQFVE KEKSAVCDEL RKKGVTICFH |
| 2041 | TKMPYISLSG PRAEVPKAAT VLEKIILSLY WKEVSIDKPG AKEIFIDRKD TLIFEAKQKF |
| 2101 | NCLIRLKEEE QQKSKNVDKV ERKLYYKLTL EDGTELLVYK GNLCNYPVDV VVNAASEDLR |
| 2161 | HTDGFAWALL QAAGPELQAE CDEVVRMTGS LQAGDAVITG AGKLPCKQVI HAIGPQWKEK |
| 2221 | NSGKCMYLLM RAIKKSLQLA ETYNHRSIAF PSVSGGIFGF PPHKCVNAIV SAIKKTLEEF |
| 2281 | KRDSSLKEIH LVAVDEETVR VLRETVQKEF TAKSSSSVLQ QQCSPNHRQG ESQREKRGDD |
| 2341 | LFMATGGENM ITTAEGLRIQ VEKKDIIDAT TDVIVNSVGT DLKFGVGPLC RALLKEAGPE |
| 2401 | LQMEFDKEKG QQVAGNGSVV CTKGYILDCT FVFHAVLPQW DRGSGQALKT LENTVHKCLM |
| 2461 | KAEEFGLKSI AFPAIGTGGF SFPHTVVSKL MFDEVFKFSR CQSRKTLQEV HFVLHPNDRQ |
| 2521 | NIQAFTSELK CRVTGKYCGA APQPDFISPV SIEASGAYTM RIGSVTLKVT SGDITKEDTE |
| 2581 | VIVNIANQTF DATSGVFKAI MDAAGFDVKE ECNQYGGLLQ SGFITTKGGA LLCRRIIHLI |
| 2641 | HSMNVKNQVS EVLHECQLRT YKSVAFPAIG TGAAQQSPAK VADDMLDAIV EFVSSRSVPH |
| 27D1 | LKEIRIIIFQ KHMLRDFLQS MKKREDPGLF EPPQSRMSTP KSCFWGPKKS IEKKKLLALE |
| 2761 | KKVDTVTFQI CGESQKNVDA TESWIIDVIL KEQLENRIVD DVIENFDERQ TEALADLQRG |
| 2821 | KHVTIKLEKN HSPPCITISG INRDVCSVFV EVQKMIQKKK AAQEEQNKAE LIYKLVGWKY |
| 2881 | QKSDDSFVAF DKLTNMQLED ARLCKKPHLT VKINKTIYKV DLNTLQAHDD QGRTINIQRE |
| 2941 | PKNGDMQSIE NPAKWEAMQG EQVKLVTLAR SCQEYLEVQK KFQKTAHNFV IEQIQRIQNP |
| 3001 | FLWQSYQIKK KSLCMKNKNQ DNEKLLFHGT AKSSLSTINY NGFNRGFAGM HAASIGKGTY |
| 3061 | FAVDAIYSAD DTYSRPDMSG RKYMYLVRVL TGEFCVGSRE LVAAPPKHSA DPTDLYDSVV |
| 3121 | DDVNAPNMFV IFNDIQAYPE YLITFRR |

Additional chicken sequences may include, but not limited to, NCBI
Genbank Reference numbers: XP_015145435.1 and XP_0151454361
XP_002942644.2 X. tropicalis (SEQ ID NO: 9) (1849 aa)

| | |
|---|---|
| 1 | MGEPSTFQFP VALQWDLGPE KLKEVKNKLL VYFQSKSKSN GGECVIRDPD CTPRYVLIHF |
| 61 | SQETVRDRVL QKQTHELSLP GGQKLKLNIS LPEAGRPNVS KAEESPAKGS AVREPKAKKA |
| 121 | RLKESPAKEP AVPLNEPSAK ETSAKKTQAQ EPPAGSKDTE NQQEPGSAAP DVKSEKANST |
| 181 | VHGTPNSMVL IENLQESYTA EMLNLLVEKD SGKAEGQDFY VEMIPEIHSA VVTFTCDIDI |
| 241 | PTFIGKLTRN HRVKKQKKLT VKVLEETKSI RVENLPPDMS DDLLEIYFEN PRYGGGEVQE |
| 301 | VELIPEENTA IITFHNIEDA KRVLGKQHVF RKQPISVYPY YESLGLTLYG EKGPCVTLPE |
| 361 | PLEVPVSPYV LEFILGDPQI KGDIDKKMAD KNCEMTWPDP NCPNPNIKLS IPSSISSHLR |
| 421 | TVAKIVRTWR DQVSTEFSLI ISKFKDAEYD VIPPVWEAIK REASSSSYGG VLVKPDLAKH |
| 481 | KVFLAGLSKV MTKVEKNFRD LVENTTRRID RQNRSMEMSE PLAPALYEIM CKTGQMETIQ |
| 541 | SQSPELRIEY DVPTRNLKLY GVKEEVLSAK CEILKETQQL KSKPLPLDPH IIHFLGFTDN |
| 601 | DELSCLFLTR HNINAMFQTE GKSVTLTGLS MKDLSEAEAQ MGQELVCKQL TVEDKMIIKG |
| 661 | PEWRSLHTHL CELFNSEKCT VVIEEFPRGA ENQVVIAGLA PSVGKASQQI HDFLERNTPI |
| 721 | QKDIPVTSAA VIQFIKEEKQ DTCMEIKKKN VSVTLKRKSI ALSGSKLYVQ EAAALIERVL |
| 781 | SSLHTNVLRI KKPGAKKFWI KNEEMYVSTA KNKFQCLVHF QKDGEDITTS LVDLSEPHCQ |
| 841 | VDLPQRVTIA VYKDDLTRHR VDVVVNAARE DLKHT [symbol] AL ALLNAAGPKL QTECDHIIKR |
| 901 | EGKYSVGDSV ITGAGNLPCK QVIHTVSPKW DPNSQTRCTR LLRRGISRCL ELAAENGLSS |
| 961 | IGIPAVGS [symbol] [symbol] FPVTVSVQ NIVESVRQYV ESPQRSRKVT RIHLVDSADG TVAAFAKAVR |
| 1021 | AEFGDYVSET SSKGNTNPDS KEPLRRSDVH MVTTKEGVNI KIIQGNIQDA TTDVIVNSVG |
| 1081 | KDLDLNTGAV SKALNAKAGT KLQQQLREMS RGTQVEEGSV FVTNGFGLNC KKVIHVVTPG |
| 1141 | WDQGKRSAEK ILRTIMTNCL STTEKEKLRS ITFPAIGTGA LGFPKDLVAS LMFDEVLKSS |
| 1201 | CKGGQLQEVN FLLHPSDMNT IKAFKDALSK RAGAEESSRQ AKDGTKAALF GLVSTPTLGV |
| 1261 | HEMRIGSLKY QVRTGDITKE STDVIVNSSN SSFTQKIGVS KAILEAAGKS IEDECATLGA |
| 1321 | QANKGYIVTQ KGNLPCRHII HVYTISTPDR IKASVLDVLQ ECENLKATSV ALPAVGTGAG |
| 1381 | GATSAAVAAA MLDAVEEFVT MKSPKSVQTV KVIVFQQKML DDFYKSMMSK EGTGVPAQSG |
| 1441 | FMGTIQSFWN YLTSKKEEPE KPKLYIPKQN IEPAVFHLCG EEKKKVMAAS SWLEELLLKE |
| 1501 | QHEDTISDAW ILEFEEHELK KLTELQKKSQ VSISIEPSGS AIRVSGLTRD VFDISKEIHT |
| 1561 | IIKEVNYRKT REREEELYSN LVEWRYHDGS GFVPFDKMSN LDLEKAQNEG KLSLTVDVGG |
| 1621 | VKYTVNMEQK SASDGKGKII GIERVCKGGL AASLPGNWDP MDKEQMKIVP LSPGSPEYNH |
| 1681 | VGGMFARSCQ MRIITIQRVQ NQYLWQNYQI KKQSIDNKTG STNNEKQLFH GTDPGTIDNV |
| 1741 | NNHGFNRSYA GKNAAVIGNG TYFAVDANYS ADDTYSKPDP SGHKHMYLAR VLTGTFTTGQ |
| 1801 | TNMITPPPKN QSNPTDLYDS VTDNINRPSM FVIFNDIQAY PEYLITFTR |

XP_004919737.1 X. tropicalis (SEQ ID NO: 10) (1499 aa)

| | |
|---|---|
| 1 | TIDIQFCLSV YFLIDAKRVL GKQHVFGKSP ISVYPYYESL GITLYGEKGP CVTLPEPLEV |
| 61 | PVSPYVLEFI LGDPQIKGDI DKKMADKNCE MTWPDPNRPN PTIKLSIPSS ISSHLRTVAK |
| 121 | IVRTWRDQVS TEWTLIISKF RAAEYDVIPP VWFAIKGEAS SSTYRGVLVK PDLAKHKVFL |
| 181 | AGLSKVMTKV EENFRALVEN TTRRIDRQNR SMEMSEPLAP ALYEIMCKTG QMETIQSQSP |
| 241 | ELRIEYDVPT RNLKLYGVKE EVLSAKCEIL KIMQQLKSKP LPLDPHIIHF LGFTDNDELS |
| 301 | CLLLTRHNIN AMFQTEGKSV TLTGLSMKDL SEAEAQMGRE LVCKQLTVED KMIIKGPEWR |
| 361 | SLHTHLCELF NSEKCTVVIE EFPRGAENQV VIAGLAPSVG KASQQIHDFL EMNTPIQKDI |
| 421 | PVTSAAVIQF IKEEKQDTCK EIKKKNVSVT LKRKSIALSG SKLYVQEAAA LIEKVLSSLH |
| 481 | TDALSINKPG AKKFFIEKEE MFVSTARSKY KCLIYLQKEG EEDFIDIDRN VDEAKCQIDL |
| 541 | PQGVTIAVYK DDLTRHRVDV VVNAANEDLK HI [symbol] ALALL RAAGPQLQTD CDRIVRTNGR |
| 601 | LYPGDAKITD GGNLPCKQVI HAVGPRWDSS SPAKCERQLR KAITNSLELA AENQHGSIGI |
| 661 | PAVSS [symbol] F PIGRCVGNIT ESISQYLEDH QGTSSIKRIH IVDTDDKIIS TFIKALKDEF |
| 721 | GDEKVQVTPK RNVTEEQKRR PNPKPEGTRE PARQMVNVNG VAIQLIKGNL QDASTDVIVN |
| 781 | SVGKDLDLDS GGASKALLRK AGKKLQHLLQ AECKGKQVQE GLVIATNGCD LSCQMVLHVI |
| 841 | APHWNQGNSV AEKTLREIIQ TCLSTTEKRQ LDSITFPAIG TGALGFPKNL VAALMFEEVE |
| 901 | KFSGSKNVQH LQEVNVVLHP SDADTMTAFF AESRKRTGTN APKANPLPKS LVSKNSGTEV |
| 961 | IGSVSTPTLG VHQMTIGSLT YQVKTGDITK ETTDVIVNSS NGNFTLRTGV SKAILEAAGQ |
| 1021 | SVADECTKLG SGPHKGFILT DKGNLLCKKI LHVCGRNSPS EIKSCVLDSL QECERIRVAS |
| 1081 | VAFPAIGSGG GSVRPEDVAG AMQAPSVAFG NYSTRCSGPK ILIFCFICLF AAFFGSLFSG |
| 1141 | SEEAEEPSVI VLKENIEPAN FSLCGEDRAQ VRAAKSWLED MVRGEQSEDV ITNDWILEFD |
| 1201 | DREQQRLRDL QRRSQVSISF ESHRSTIRVS GLTRDVFNVS KEIHEMVKKI SVRKTREREA |
| 1261 | ELYSNLVEWR YHDGSGFAPF DKMSNLDLEK AQNEGRQSLT VDVGGVKYTV NMERKSASDP |
| 1321 | RGKNVKIDRA SKSGETMELP PDWDTMTNEQ VKVVPLNPQT PEYRDVQGLF AKSCPMTIIK |
| 1381 | VTVCIRAATS YINYDVSVFL SGAAACYGNG TYFAVNASYS AHDTYSRPDT NRHKHMYLAR |
| 1441 | VLTGVSCVGR QGMVAPPAKN PSNPTDLYDS VTDRAANPNM FVIFNDIQAY PEYLITFTQ |

XP_004919736.1 X. tropicalis (SEQ ID NO: 11) (1848 aa)

| | |
|---|---|
| 1 | MGEPTAFQFP VALKWDLGPE KLKELKNKLL VYFQSKSKSN GGECVIRDPD CTPEYVLIHF |
| 61 | SQETVRDRVL QRQTHELSLP GGQKLQLNIS LPEAGRPNIS KAEESPAKGS AVKEPKAGDA |
| 121 | KEPPYMEPAP KETLVIEPPT KETSGKKTQA QEPPAGSKDA DIQQEPGSAA PDVKSEKANS |
| 181 | SVHGTLNSMV LIENLPESHT AEMLNLLVEK DSDKAEGQDF YVEMIPEIRS AVVTFTCDID |
| 241 | TPSFMGRLGR NPRFKQKKLT VKALEETNSI RVENLPPGTD DHVVNYFENP RHGGGEVQEV |
| 301 | KLIPEEDAAI VTFNNVEDTY KVLAKQHVFG KKPVSVYLYY LPLGITLYGK EGPCVTLPEP |
| 361 | LEVPVSPYVL EFILGDPQIK GDIDKKMADK NCEITWPDPN CPNPTIKLSI PSSISSHLRT |
| 421 | VAKIVRTWRD QVSTEWTLII SKFRAAEYDV IPPVWEAIKG EVSSSTYEGV LVKPDLAKHK |
| 481 | VFLAGLSKVM TKVEQTFKDL VENTTRQIDR QNRSMEMSEP LAPALYEIMC NSGHIKNIQS |
| 541 | QSPELRIEYD VPTRNLKLYG VKEEVLSAKC EILKETQQLK SKPLPLDPHI IHFLGFTDND |
| 601 | ELSCLLLTRH NINAMFQTEG KSVTLTGLSM KDLSEAEKQM GRELVCKQLT VEDKMIIKGP |
| 661 | EWRSLHTHLC KLFNSEKCTV VIEEFPRGAE NQVVIAGLAP SVGKASQQIH DFLERNTPIQ |
| 721 | KDIPVTSVAV IQYIMQEKKQ LYEEIKRKNV NVGMKEKTIT LSGSKLYVQE AAALIEKVLS |
| 781 | SLHSDIVCIK KPGAKKFCIE KEEMYVSAAK NKFQCLVHFQ KDGEDITTSL VDLSEPHCQI |
| 841 | DLPQGVTIAV YKDDLTRHRV DVVVNAARED LKLT [symbol] ALD LLSAAGPKLQ TECDHIIKRE |
| 901 | GKYSVGDSVI TGAGNLPCKQ VIHTVSPKWD PNSQTRCTRL LRRGISRCLE LAAENGLGSI |
| 961 | GIPAVGS [symbol] [symbol] FPVTVSVQS IVESVRQYVE STERSRKVTR IHLVDSADGT VAAFAKAVRA |
| 1021 | EFGDHVPETS SKGNTNPDSK EPPRRSDGQM ATTKEGVNIK IIQGNIQDAT TDVIVNSVGK |
| 1081 | DLDLNTGAVS KALNAKAGTK LQQQLREMSR GTQVEEGSVF VTDGFGLNCK KVIHVVTPGW |
| 1141 | DQRMGSAEKA LRGSLESCLS TTEKDKLRSV TFPAIGTGAL GFPKHLVASL MFDEVLKFSC |
| 1201 | KHKGGQLQEV NFLLHPSDTE TIKAFNGELS ERTKTEESSH QSKDGTKAAL FGLVSTPTLG |
| 1261 | VHEMRIGSLK YQVRTGDITK ESTDVIVNSS NSSFTLKAGV SQAILEAAGK SVEDECTKLG |
| 1321 | AQANKGYIVT QKGNLPCRHI IHVHAISTPD KIKASVLDVL QECENLKATS VALPAMGTGA |
| 1381 | GKATSAAVAA AMLDAVEEFV TTKSPKSVQT VKVIVFQQKM LDDFYKSMMS KEETGLLARS |
| 1441 | GFMGTIQSFW NYLTSKKEEP EKPKLYIPKQ NIEPAVFHLC GEEKKKVMAA SSWLEELLLK |
| 1501 | EQHEDIISDA WILEFEEQEL KKLTELQKKS QVSISIEPSD SAIRVLGLTR DVFDISKEIH |
| 1561 | LIIKEVNYRK TRERDAELYS NLVEWKYHNG SGFVPFDKMS NLDLEKAQNE GKLSLTVDVG |
| 1621 | GVKYTANMER KSVSDGKGKI IGIERVCKSG LALPGNWDPM DKEQMKIVPL SPGSAEYNQV |
| 1681 | EGMFARSCQM RIIKIQRVQN QYLWQNYQIR KQSIDTKSSS TNNEKQLFHG TNPGTIDNVN |
| 1741 | NHGFNRSYAG KNAAAIGNGT YFAVDANYSA NDTYSKPDPS GHKHMYLARV LTGTFTTGQK |
| 1801 | HMITPPSKNQ SNPTDLYDSV TDNINRPSMF VIFNDIQAYP EYLITFTR |

NP_001107919.2 D. rerio (SEQ ID NO: 12) (1810 aa)

| | |
|---|---|
| 1 | MDNAEFPYAL CVEGQWDPRT PKLKNKFIIY FQSKKSDGGD CVVEYSVSDG QTATVRFKTE |
| 61 | QVRRRVVNKQ THELKIGQKT VKITVSLPSS EASDPQEATS SVSTITAGEP LREEEKTTES |
| 121 | SEENEEDTTI RSAVIENIQN SNQEFLTILV ENVLRGSSTE SNDFSIEIIP ESNCAVVTFS |
| 181 | NRKDTETFII SCQSNAMIKK KNMKVGLLEK TCKVKAEGLP PNTNSDYLML YFEKFGEIED |
| 241 | DGVTVDTDES AIITFANPED VQKVIKLSHQ IKKHPFKVLP YNEKLQTALY GKDRPTLKLP |
| 301 | EAFTEKMDRN VWHHLKENQI SLDLVKQAMS CHFCVMDFQS SEVKISPLPS LLHQGEQTRK |
| 361 | LIQTWRENAL SGFTMSMSKF KSVELHIQKD AWTDVQPEIY ETVAEEAVTL VPYADEGTIA |
| 421 | VAGLKEQVND IERTLKETVQ RITQKIQREK GSESDEISMT PSIYNLVLCD GLKQETNDKF |
| 481 | PEMDITYLVH SQKLTLYGLK DEVLETKNKI LEAVLHLIRK PTELNQSVLD FLLKKDLEEM |
| 541 | TKDLFFNKGV NVALEADGQR VLLVGKTDKD LSVGEKQLQT DLGHLSFNVD DPSVFRRSDW |
| 601 | QDLVSNIKNN FRTVAVQTCA NKVVVSGFVD SINDVEQQLY DFVQENSHME KTLNAHKTVI |
| 661 | TFIKDHRKQD WFEEVKGKVS VDFKNDSIVI SGPKLHVCDC VPLFDNLCTS AHHCTMKIVK |
| 721 | PGAKNFFKEK EPMCVSYAKT NMNCLVQLIN ESDHPHDTVE PRKVKSVYQF KTREGMEITV |
| 781 | HKADMCSFQV DAVVGACKET LLLD [symbol] AKA LSDAAGPKLQ KDCDKLVKGR KFTTGDAVLL |
| 841 | DAGGRLHCKH VILAIGPHYN SSKPQESEKL LKKAVKRSLN VADQESFQSI AIPAISS [symbol] |
| 901 | [symbol] FPMDLCAFT IVKAIKEFCD FVEGDIALTK IHLVDNNDKT VQALEAAVKK VYGVSDQSTT |
| 961 | GSSSSSQQQN KASASPSQHQ LTTFFQGASQ TESFQTKEGL TITLMKGNIE DTTMDVVVNT |
| 1021 | LSSDLKLNVG AVSNALFKAA GPQLQDLLDQ QATGPASSGA VFETAGANLK NKLVFHAVVP |
| 1081 | HWNQGQGNEQ KVLENVMDTC LCKAEQRQQS SIVFSAIGTG NLGFPKSLVV STMLDSVFKF |
| 1141 | STKRSSKHIQ EVAFVLHPKD TQTIQVFTDE FSKRFLGQSA SASNSTQPQS TGPFSKVTTK |
| 1201 | SGIHETTVGG VTLQVLNGDI TTEQTDVIVN STTKEFTLKA GVSKAILDKA GPNVEAECQQ |
| 1261 | LGAKTDSGLI MTQAGNLQCK KIIHIAAQSN AIMIQKHVRK ALEMCAKEKL TSIAFPAIGT |
| 1321 | GQAGLSPGQV ADSMLDGMLE MLRKTPQSSL KLIRLVVFQA HMLPEFLKSM QNRETGPAKQ |
| 1381 | EKESTWSKIK AFAATAKSYL TGSWEKEIKQ HGGKDFVIDG VEVDPVFFSI CGPSKAEVDQ |
| 1441 | AKIFLEDMIT KEQVFQSITD TAILSLSDKD QQRILDLQST IDVTIRLEYK ANKATDESPG |
| 1501 | KATLIVQGLS RDVLKATHEI NEMLKSAKEN EILKKEMNLM SELVDWQFEQ GGQYKSFDER |
| 1561 | TNLELEKALS RQAADTSITF QGQTYQVKLP EGPAVNNAGG NQMNIRRIDK LKATESIPQH |
| 1621 | WDAMGSNELN KKFNLQPTST EYQDVLGRFK ATCRNNVLKI ERVQNPGMWK NYLNNKSVME |
| 1681 | KKNGHQNNEK RLFHGTSEQT ISHIEKSGFN RSYAGKNAAA YGNGTYFALN ASYSASNTYS |
| 1741 | VPNAQGHKHM YLCRVLAGDY TKGQGGMIVP PAKNANCDLY DTVVDNPAAP TIFVVFRDDN |
| 1801 | AYPEYLITFT |

XP_002663580.3 D. rerio (SEQ ID NO: 13) (1815 aa)

| | |
|---|---|
| 1 | MEEYPIVVEG DWGPEHAKIV KNKLQIHFQS KKKSQGGDCV VKFNDKSNSA TILFKSSDIR |
| 61 | DAVLSQEAHV VTIETLQIKL NVYKPSDGQE QADSTVLSTD QACGYQDPTQ TSLQIELDEK |
| 121 | KTEESSAVVL ENLPDDFNED VLTLFVENIT TLTKNDFTVE FIPELSKAVV TFKNPSAAEK |
| 181 | FLEESETHKK FKKNNLKARA LERSTCVRVE NLPAEVNNKK MLLELYFEKL GGPVDEVITI |
| 241 | PSEQAAIITF KEEEARERVL KQENNICDVP VKIYPYFKSL DTVLYGGNRP HFKIPEPITV |
| 301 | RVHPAIREFL LKKGQVSSIE DKMSSHFCLI NMDKPDVLLS PDPALLKQKG VSRRHIDGWS |
| 361 | KNALDAFKKI ISDYTTSEWP VSHALWSKVE TDVKTVVKDQ VYTDMDVSNG VLTLAGMSHE |
| 421 | IIGLKPIMEK ILERATNQLE REKNSVTENI EMSPAMYSLL EQDGLKSAKV VSPQLRIEYN |
| 481 | QKLSTLVLSG LQTEILLFKN WFLEKKLSMK MKPLNISHPV LDFLKSVDCD EMSTDLFLSH |
| 541 | RISAVYTIEN GDVVVIGSTE QAHAEAEKRL NTVLTSKALL VEDHSVLQMP EWQDLKKQLL |
| 601 | ELFNTSKKRT VSINVSQRDK VIVTGFREPV MEVSESLEHF IEKHTRIEEM VRVKSHAAVE |
| 661 | FIEDRKSQDW QRFMKSGEIK VSFDPKRPRI KLSGQRIFVQ PAVTFFKGLA ESLLTHTLTI |
| 721 | KKAGAKKYFK EQGKMMLLML LKDKRVVVVL QEDHMLEEDE DRPKEGNFGD FGQASCEVRM |
| 781 | PGGVTVTVRK ADICTLSVDA VVNAANEDLK HT [symbol] AYALL QAAGRCLQEY CDLHIKVNGP |
| 841 | LTPGDAIITD AGRLPCKYVV HAVGPRFGAL DQRTAVQQCL RRAVRESLNQ ASSKKCSSIA |
| 901 | IPVISS [symbol] CPLDLCTESI AKEVRQYIEN HNYSGSNSTL TEIQLVDNNS KNVNAMAQAV |
| 961 | RNEFAAYNPK MAFPHQSKPH EYSNYEHGHR GGGRVHGHGH ENSTKRNQQF ESPTHRGNRD |
| 1021 | FKEQSNSWSG GRSDNSEELT FLESKTTQDG LKIILSKGNI QDASADVIVN TISHDMDLSK |
| 1081 | GAVSNALLQC AGHQLQSEIS RAAPSSGLSY GDLVITAGYK LKCSKVFHVV CPFWHGGQNS |
| 1141 | PDKVLVQIIK DCLGEAETRG MTSVVFPAIG TGNLGFPKDL VAKIMLTEVQ QFNTTNLRKV |
| 1201 | TVVVHSFDKE MSSGIVGTVS SPSLGVHIMQ LGQVTLEVSS GDITKENTAA IVNSSDKTFS |
| 1261 | LKAGVSKAIL DAAGVQVEQE CSQIVASSYS QQTEILTSAG QLPCGNIIHI IGRNNPSDIK |
| 1321 | GVVLSVLKIC ESQQFTSIAF PALGTGQGGA NPADVADAMV DAVVDFVKKK KPVHVKVVKF |
| 1381 | LIFQASMLSH FHQSMVRRSG EKVEEDRGLL TKLKDFFSWG SSETPPSEEF VMVSEDIEPA |
| 1441 | VFQLCGETPQ DLSEAKEMIN NLIIREHLTN PIRDPAIAHF TREDVEMLNA MQKELTVSFK |
| 1501 | LEKKGQDSVV TLQGLTRDVH TAESRVRDMI RKVERNENRR REALMIRAVV KWQYQENGQR |
| 1561 | FENFDLLTNF DLEQAFQNRQ PSVKIKINND EYEADLVRKE ARRGRIRIEL NRIDLEDAAQ |
| 1621 | SSLPSHWEDM TGQPVVLVKL KTDSKEYAEV EKKFTSTGLS NIIKIQRVQN STLWRNYKIK |
| 1681 | KEELEDKNKH TNNERHLFHG TGPDTTDQIN HHGFNRSYAG MHGAMFGKGS YFAVDPSYSA |
| 1741 | QGYSKPDAKG HKRMYLAKVL VGDFTRGNPG LLVPPAKSSS SADLYNSVTD NVNNPTMFVI |
| 1801 | FNDVQAYPEY LITFQ |

XP_691115.6 R. rerio (SEQ ID NO: 14) (1890 aa)

| | |
|---|---|
| 1 | MHEKIKKNNL KVRSLERSTC VRVEDLPAEA NEMLLELLFE KWGGPIQQID TIPSEHAAII |
| 61 | TFKDEEAKER VLKQQNMISN VPVKMYPYFK SLDMVFYGDN KPHLKLPEPI TVSVHPAIRA |
| 121 | FLLKKGLIFS IEDKMSSHFC LINMDKPDVL LSPDPALLKQ KGVSRRHIDG WSKNALDAFK |
| 181 | KIISDYTTSE WPVSHALWSK VETDVKAVVK DQVYTDADTS KGVLTLAGMG HEIIGLKPIM |
| 241 | EKILERATNQ LKREKNSVTE NIEMSPAMYF LLEQDGLKTA ASSQLRIDYF KNMNRLTVSA |
| 301 | KEKVLKQQIF ISNIPVKIYP YFKSLDMVFY GDNKPHLKLP EPITVSVHPA IREFLLKKGQ |
| 361 | ISSIEDEMSS HFCQINMDKP DVLLSPDPAL LKQKGVSRRH IDGWSKNALD AFKKIISDYT |
| 421 | TSEWPVSHAL WSKVENDIKT VVKDQVYTDM DAFKGVLTLA GMSHEIIGLK LIMEKILEKA |
| 481 | TNQLEREKNS VTENMEMSPA MYFLLKQDGL KTAASPQLRI DYYKNMNRLT VSGLQKESLV |
| 541 | FKNWVLQKQI NIKQKSLKID RSVLDFLKSV DCDEMSTDLF LSHRISAVYT IENGDVFVIG |
| 601 | STEQACTEAE KRLNTVLTSK ALLVEDHSVL QMPEWQDLKK QLLEFNTSKK RTVSINVSQR |
| 661 | DKVIVTGFRE SVIEVSESLE KFIEKHTRIE ETVRVKSHAT AQFIKDIKSQ DWQRFMKSDE |
| 721 | IKVSFDPKRA RIKLSGQRIF VQPAVTFFKG LAESLHMDTL TIKKAGAKKY FKEQGKMMLL |
| 781 | MLLKDKRFVV LLQEDHMREE DEDDFTESNF GDFGQASCEV RMPGGVTVTV RKADICTLSV |
| 841 | DAVVNAANED LKHT [symbol] AFA LLQAAGRCLQ EYCDLHIKVN GRLSPGDAII TEAGRLPCKY |
| 901 | VVHAVGPRFR GSDKRTAVQQ CLRRAVRESL NQASSKKCSS IAIPVISS [symbol] CPLDLCTE |
| 961 | SIAKEVRQYI ENHNYSGSNS TLTEIQLVDN NDKNVNAMAQ AVRNEFAAYN PKMAFPHQSK |
| 1021 | PHEYSNYEHG HRGGGRGRGH GHGHGHENYN QRNHRGHSNR EFEEQAHSQS GDRTDNSEGL |
| 1081 | RVLETKTTQD GLKICLYEGN IQDASADVIV NTISEDMDLR KGAVSKALLQ AAGHQLQSEV |
| 1141 | NRAAKSFRLN TGEIVITDGY NLKCSRVFHV VCPFWSQGSE DKVLCQIIRN CLKNAETQRM |
| 1201 | NSIVFPAIGT GNLGFPKDLV AKIILTEVQQ FNTTNLRKVT VIVHPSDKES VQCFISIFRH |
| 1261 | GYQGPITKEA HQHFKPKKSA QTSGTHGKVS SPSLGVYIMQ LGQVTLEVSS GDITKEKTDA |
| 1321 | IVNSSNPSFS LKAGVSKAIL DAAGGKVEHE CSQIVASQNK QLREIVTSSG RLPCGKIIHI |
| 1381 | SGCNGLSTIK EVVLSVLKLC ESQQFTSIAF RALGTGQGGA EPGDVADAMV DAVVDFVKKK |
| 1441 | KPVHVKFVKF LIFQPKMLAD FHQSMVRRSG EKVEEEKGLF TKIKDYFLGE SSESSANEEF |
| 1501 | VMVGEEIEPA VFQLCGETPQ DLSEAKEMIN NFIIREHLTI PIHDPAIAYF TREDVEMLNA |
| 1561 | MQKELTVSIK LERKGQDSVV TMEGLTRDVH TAESRVRDMI RKVERNESKK REALMIRTII |
| 1621 | KWQYQENGQR FENFDMLTNF DLEQAFQNQQ PSVKIKINND EYEADLVSKE AKRRGIRIKL |
| 1681 | NRVDLEDTAE AASQSSLPSH WENMKGNSVI LVKLKAGSKE YTEVEKEFRR TGLTFKIIEI |
| 1741 | ERVQNSALWR NYMIKMNELE DKNQHKNNER RLFHGTGSDT TDLINNHGFN RSYAGMHGAM |
| 1801 | YGKGSYFAVD PSYSAKGYSK PDAKGHKRMY LAKVLVGDFT RGNSGLLVPP AKSSSSADLY |
| 1861 | NSVTNDVNNP TMFVIFHDVQ AYPEYLITFQ |

Included in Table 1 are deletion of, or mutation to, one or more amino acids, individually or combined, within the Macro domain 1 as noted in bolded, italicized, highlighted, and underlined. Such mutations may abolish PARP14 activity (e.g., NADase activity, ADP-ribose hydrolase, mono(ADP-ribosyl)transferase activity, and/or poly(ADP-ribosyl)transferase activity). Mutations may include any one of substitutions (conservative or non-conservative), insertions, or combinations thereof that may result in loss of NADase activity, ADP-ribose hydrolase, mono(ADP-ribosyl)transferase activity, and/or poly(ADP-ribosyl)transferase activity. Additional mutations, include mutations, substitutions, or deletion of the phosphorylation site (bolded and underlined) between Macro Domain 3 and the PARP domain (see FIG. 6). For example, mutation of Serine 1419 of SEQ ID NO: 6, or Serine 1403 of SEQ ID NO: 1, may abolish PARP14 NADase activity.

Also included in Table 1 are polypeptide molecules comprising, consisting essentially of, or consisting of:
1) an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of SEQ ID NO: 1-14, or a biologically active fragment thereof;
2) an amino acid sequence of SEQ ID NO: 1-14 having at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, or more amino acids, or any range in between, inclusive such as between 1400 and 1900 amino acids; or
3) nucleotide sequences encoding any of the polypeptides or amino acids set forth in 1) to 2) above.

Also included in Table 1, are the nucleotide sequences encoding the PARP14 polypeptides, or homologs thereof. For example, the nucleotide sequence encoding NP_060024.2 is set forth in GenBank accession NM_017554.2; the nucleotide sequence encoding XP_516695.3 is set forth in GenBank accession XM_516695.5; the nucleotide sequence encoding XP_001105869.2 is set forth in GenBank accession XM_001105869.3; the nucleotide sequence encoding XP_850880.2 is set forth in GenBank accession XM_845787.5; the nucleotide sequence encoding NP_001193467.1 is set forth in GenBank accession NM_001206538.1; the nucleotide sequence encoding NP_001034619.2 is set forth in GenBank accession NM_001039530.3; the nucleotide sequence encoding NP_001178588.1 is set forth in GenBank accession NM_001191659.1; the nucleotide sequence encoding XP_422113.4 is set forth in GenBank accession XM_422113.4; the nucleotide sequence encoding XP_002942644.2 is set forth in GenBank accession XM_002942598.4; the nucleotide sequence encoding XP_004919737.2 is set forth in GenBank accession XM_004919680.2; the nucleotide sequence encoding XP_004919736.1 is set forth in GenBank accession XM_004919679.2; the nucleotide sequence encoding NP_001107919.2 is set forth in GenBank accession NM_001114447.2; the nucleotide sequence encoding XP_002663580.3 is set forth in GenBank accession XM_002663534.3; and the nucleotide sequence encoding XP_691115.6 is set forth in GenBank accession XM_686023.8.

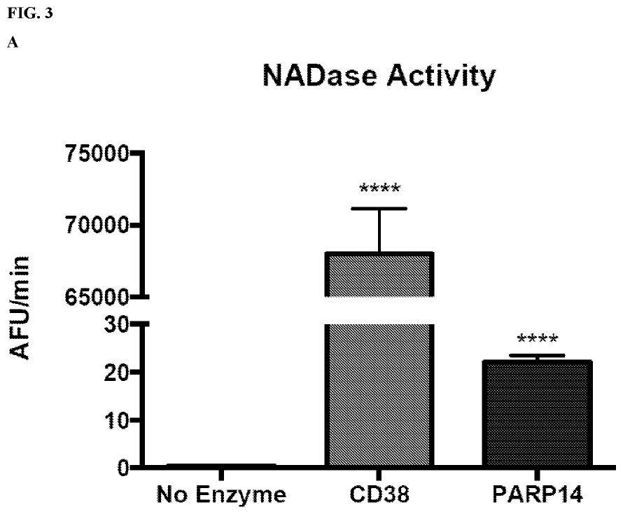

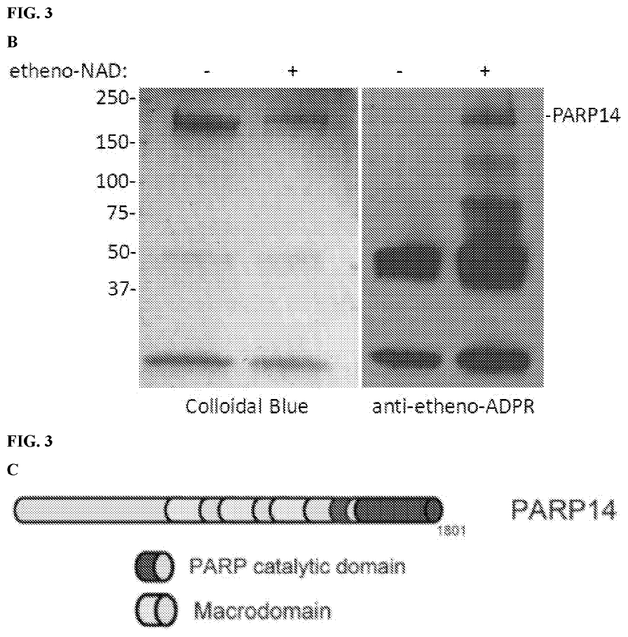

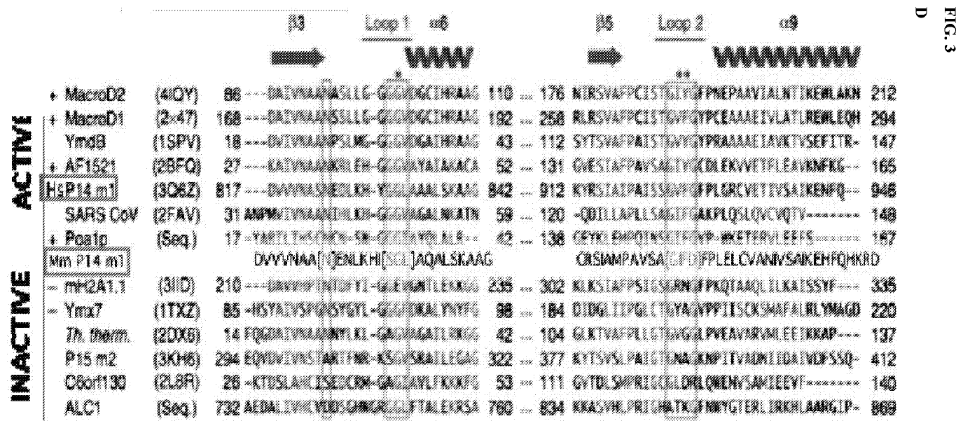

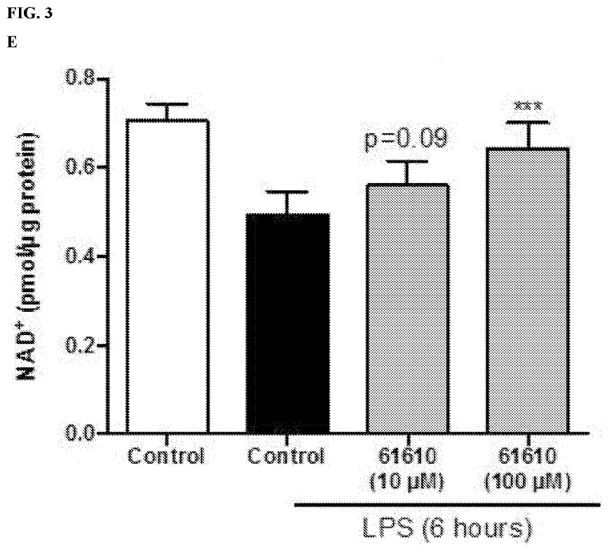

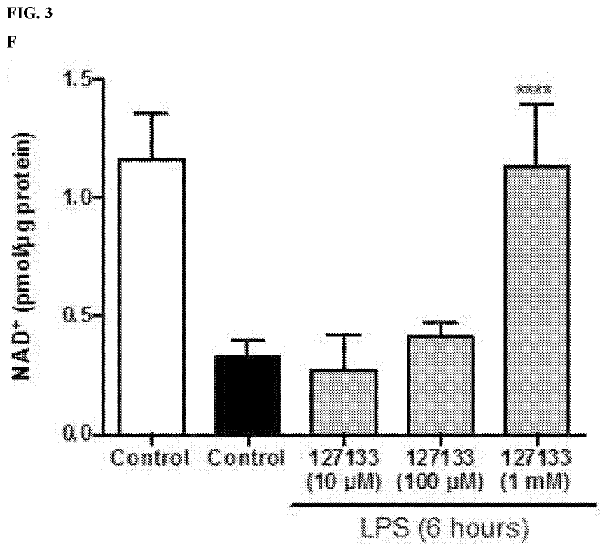

A comparison of human and mouse PARP14 Macro domain 1 sequences to multiple alignments of known catalytically active and inactive Macro Domains from various species are shown in FIG. 3D. (adapted from Jankevicius, Nat. Struc. and Mol. Biol., 20 (4):508-14 (2013)).

Table 2 provides a multiple amino acid sequence alignment for SEQ ID NOs:1-14. The predicted consensus Macro domain 1 sequences are bolded and underlined.

TABLE 2
Multiple sequence alignment for SEQ ID NOS: 1-14 depicting predicted
consensus Macro Domain 1 sequences. Bolded, italicized, underlined,
and highlighted amino acids reflect key amino acids in Macro Domain 1.
Deletion of, or mutation to, one or more amino acids, individually
or combined, may abolish PARP14 activity.

| | |
|---|---|
| zebrafish_2 | ----EDRPKEGNFGDFGQASCEVRMPGGVTVTVRKADICTLSVD-AVVNAANEDLKHT [symbol] |
| zebrafish_3 | ----EDDFTESNFGDFQGAWCEVRMPGGVTVTVRKADICTLSVD-AVVNAANEDLKHT [symbol] |
| zebrafish_1 | ----------TVEPRKVKSVYQFKTREGMEITVHKADMCSFQVD-AVVGACKETLLLD [symbol] |
| frog_2 | ----------DIDRNVDEAKCQIDLPQGVTIAVYKDDLTRHRVD-VVVNAANEDLKHI [symbol] |
| frog_1 | -----------SLVDLSEPHCQVDLPQRVTIAVYKDDLTRHRVD-VVVNAAREDLKHT [symbol] |
| frog_3 | -----------SLVDLSEPHCQIDLPQGVTIAVYKDDLTRHRVD-VVVNAAREDLKLT [symbol] |
| chicken | ---------QREHSNVGKCYTQMT-VHGVLIAVYKADLCTHHVD-VVVNASNEDLKHI [symbol] |
| mouse | GEEEEGEEEGESSINEQKCHLQRDIAPGVKLFVLEEDLSRFPVD-VVVNAANENLKHI [symbol] |
| rat | ------EEGEEDSLKEQKCYLQRDIGPGVTLIVHEGDLSLFPVD-VVVNAANESLKHI [symbol] |
| dog | ----------AGRGDAHRGLPRVELRPGVALAVQRGDLTRFPAAQAVVSPASGDLQLR [symbol] |
| cow | ---------EGRGTGGQRCFFRTDLAPGVSLIVQQGDLTHFPVQ-VVVNAANEELKLR [symbol] |
| rhesus | ---------EGGSPSGQKCFSRTVLAPGVVLIVQQGDLAQLPVD-VVVNASNEDLKHY [symbol] |
| human | ---------EGGSPAGQKCFSRTVLAPGVVLIVQQGDLARLPVD-VVVNASNEDLKHY [symbol] |
| chimp | ---------EGGSPAGQKCFSRTVLAPGVVLIVQQGDLAQLPVD-VVVNASNEDLKHY [symbol] |
| | . . : : * . *: . .**. . * * |
| zebrafish_2 | [symbol] AYALLQAAGRCLQEYCDLHIKVNGPLTPGDAIITDAGRLPCKYVVHAVGPRFGALDQRT |
| zebrafish_3 | [symbol] AFALLQAAGRCLQEYCDLHIKVNGRLSPGDAIITEAGRLPCKYVVHAVGPRFRGSDKRT |
| zebrafish_1 | [symbol] AKALSDAAGPKLQKDCDKLVKGRKFTTGDAVLLDAGGRLHCKHVILAIGPHYNSSKPQE |
| frog_2 | [symbol] ALALLRAAGPQLQTDCDRIVRTNGRLYPGDAKITDGGNLPCKQVIHAVGPRWDSSSPAK |
| frog_1 | [symbol] ALALLNAAGPKLQTECDHIIKREGKYSVGDSVITGAGNLPCKQVIHTVSPKWDPNSQTR |
| frog_3 | [symbol] ALDLLSAAGPKLQTECDHIIKREGKYSVGDSVITGAGNLPCKQVIHTVSPKWDPNSQTR |
| chicken | [symbol] AWALLQAAGPELQAECDGVVRMSGSLQAGDAVITGAGKLPCKQVIHAVGPRWKEQDAEK |
| mouse | [symbol] AQALSKAAGPELQTECDQIVKEGGVVLPGNAVISKAGKLPCHHVIHAVGPRWKGDKVLE |
| rat | [symbol] AGTLSKAAGPSLQAECDLIVKKGGMVPPGNAVISKAGKLPCRYVIHAVGPRWKGDKVLE |
| dog | [symbol] AAALAQAAGPALQEDCSRLLRTAGPVPVGGAVASTAGKLPYRLVIHAVGPQWKDSEASR |
| cow | [symbol] AAALLRAAGPELQEDCDQILKKRGKLSPPYAVISKAGKLPYQHVIHAVGPQWKSDEAQK |
| rhesus | [symbol] AAALSKAAGPELQADCDQIVKREGRLLPGNATISKAGKLPYHHVIHAVGPRWNGCEAPR |
| human | [symbol] AAALSKAAGEPLQADCDQIVKREGRLLPGNATISKAGKLPYHHVIHAVGPRWSGYEAPR |
| chimp | [symbol] AAALSKAAGPELQADCDQIVKREGRLLPGNATISKAGKLPYHHVIHAVGPRWSGYEAPR |
| | :* * *** ** *. :: .*. : *: ::.*:: . |
| zebrafish_2 | AVQQCLRRAVRESLNQASSKKCSSIAIPVISS [symbol] CPLDLCTESIAKEVRQYIENHNYS |
| zebrafish_3 | AVQQCLLRAVRESLNQASSKKCSSIAIPVISS [symbol] CPLDLCTESIAKEVRQYIENHNYS |
| zebrafish_1 | SE-KLLKKAVKRSLNVADQESFQSISIPAISS [symbol] FPMDLCAFTIVKAIKEFCDFVE-- |
| frog_2 | CE-RQLRKAITNSLELAAENQHGSIGIPAVSS [symbol] FPIGRCVGNITESISQYLEDHQ-- |
| frog_1 | CT-RLLRRGISRCLELAAENGLSSIGIPAVGS [symbol] FPVTVSVQNIVESVRQYVESPQ-- |
| frog_3 | CT-RLLRRGISRCLELAAENGLGSIGIPAVGS [symbol] FPVTVSVQSIVESVRQYVESTE-- |
| chicken | CV-YLLKKTIKKSLQLAETYNHRSIAFPSVSG [symbol] FPLHKCVNAIVSAIKKTLEEFK-- |
| mouse | CV-SLLKKVVRQSLSLAEEHRCRSIAMPAVSA [symbol] FPLELCVANIVSAIKEHFQHKR-- |
| rat | CV-NLLKKAVKQSLTLAEEYKCQSIAIPAISS [symbol] FPLDLCVASIASAIKDNVQQKQ-- |
| dog | CV-SQLKAAVKQSLRLAEAHGCRSVAIPAISS [symbol] FPLAVCVRTIVLAVQESCQLSH-- |
| cow | CV-LQLKNAVKESLNLAVKHKYQSIAIPAISS [symbol] FPLSQCVEAIVLAIKEWFQCKK-- |
| rhesus | CV-YLLRRAVQLSLCLAEKYKYRSIAIPAISS [symbol] FPLGRCVETIVSAIKENFQRKK-- |
| human | CV-YLLRRAVQLSLCLAEKYKYRSIAIPAISS [symbol] FPLGRCVETIVSAIKENFQFKK-- |
| chimp | CV-YLLRRAVQRSLCLAEKYKYRSIAIPAISS [symbol] FPLGRCVETIVSAIKENFQFKK-- |
| | . *: : .* * *:.:* :.. *. .. *. : . : . |
| zebrafish_2 | GSNSTLTEIQLVDNNSKNVNAMAQAVRNEFAAYNPKMAFPHQSKPHEYSNYEHGHRGGGR |
| zebrafish_3 | GSNSTLTEIQLVDNNDKNVNAMAQAVRNEFAAYNPKMAFPHQSKPHEYSNYEHGHRGGGR |
| zebrafish_1 | -GDIALTKIHLVDNNDKTVQALEAAVKKVYGVSDQSTTGSSSSSQQQNKASA-------- |
| frog_2 | -GTSSIKRIHIVDTDDKIISTFIKALKDEFGDEKVQVTPKRNVTEEQKR----------- |
| frog_1 | -RSRKVTRIHLVDSADGTVAAFAKAVRAEFGDYVSETSSKGNTNPDSK------------ |
| frog_3 | -RSRKVTRIHLVDSADGTVAAFAKAVRAEFGDHVPETSSKGNTNPDSK------------ |
| chicken | -RDSSLKEIHLVDITEDNVQAFIKALKEVFSDDVPLNNPVHQTSIVHR------------ |
| mouse | -DTHTLKKIYLVGLPAKVARAFAEAVKTTYKDSLSHTAFPSSLK-ALV------------ |
| rat | -NTHSVKKIYLVDVSAKVAKAFAEAVKTTYRDILS----PASVK-ALV------------ |
| dog | -DGHALKEIYLVDTAEKTVQAFADTVTALAE--------VPG-PAAVQ------------ |
| cow | -DGCTLKEIYLVDIAEKTVEAFAESAKSVFKDTLPDIASLPSLPAAAQ------------ |
| rhesus | -DGHYLKEIYLVDVSEKTVEAFAEAVKTVFKATLPDTAAPPSLP-AAA------------ |
| human | -DGHCLKEIYLVDVSEKTVEAFAEAVKTVFKATLPDTAAPPGLPPAAA------------ |
| chimp | -DGHCLKEIYLVDVSEKTVEAFAEAVKTVFKATLPDTAAPPGLPPAAA------------ |
| | :..* :*. :: : |

In some embodiments, PARP14 mutants/variants may include deletion of, or mutation to, one or more amino acids, individually or combined, within the Macro domain 1 as noted in Table 2 (bolded, underlined, italicized, and highlighted amino acids). Such mutations may abolish PARP14 activity (e.g., NADase activity, ADP-ribose hydrolase, mono(ADP-ribosyl)transferase activity, and/or poly(ADP-ribosyl)transferase activity).

Mutations may include any one of substitutions (conservative or non-conservative), insertions, or combinations thereof that may result in loss of NADase activity, ADP-ribose hydrolase, mono(ADP-ribosyl)transferase activity, and/or poly(ADP-ribosyl)transferase activity.

TABLE 3
Multiple sequence alignment for SEQ ID NOs: 1-14 depicting
phosphorylation site key for NADase activity
(bolded and underlined amino acid)

| | |
|---|---|
| zebrafish_2 | FKLIFQASMLSHFHQSMVRRSGEKVEE-----DRGLLTKLKDFFSW----------GSSE |
| zebrafish_3 | FKLIFQPKMLADFHQSMVRRSGEKVEE-----EKGLFTKIKDYFLG----------ESSE |
| zebrafish_1 | RLVVFQAHMLPEFLKSMQNRETGPAKQE----KESTWSKIKAFAATAKSYLTGSWEKEIK |
| frog_2 | KIL-----------------------------IFCFICLFAAFFGS----LFS----GSE |
| frog_1 | KVIVFQQKMLDDFYKSMMSKEGTGVPA-----QSGFMGTIQSFWNY----LTS----KKE |
| frog_3 | KVIVFQQKMLDDFYKSMMSKEETGLLA-----RSGFMGTIQSFWNY----LTS----KKE |
| chicken | IIVIFQPHLLNVFHTSMKKRENDGKTK-----SKSLISRVTSFLIP-----------EKH |
| mouse | KVVIFQPHILQFFYDNMKEREGSPAPPKQSPAKQSVMSKIASFLGF-----------PKQ |
| rat | KVVIFQPHILQFFYDNMKEREGSPAPV-----KPSVMSKIASFLGF-----------PTQ |
| dog | KVVIFLPHLLDVFYDSMKKREASQASP-----QQSLESKLKSSVSS-----------PSQ |
| cow | KVVIFQPQVLDVFRANMTKREGYQASF-----QQSVISKIASFLGF-----------SSK |
| rhesus | KVVIFLPQVLDVFYAIMKKREGTQVSS-----QQSMMSKLASFLRI-----------SKQ |
| human | KVVIFLPQVLDVFYANMKKREGTQLSS-----QQSVMSKLASFLGF-----------SKQ |
| chimp | KVVIFLPEVLDVFYANMKKREGTQLSS-----QQSVMSKLASFLGF-----------SKQ |

In some embodiments, PARP14 mutants/variants may include mutations, substitutions, or deletion of the phosphorylation site (bolded and underlined) between Macro Domain 3 and the PARP domain (see FIG. 6). For example, mutations of Serine 1419 of SEQ ID NO: 6 or Serine 1403 of SEQ ID NO: 1 may abolish PARP14 NADase activity.

As used herein, the term “decreased” and grammatical equivalents thereof refer to a level, amount, concentration of a parameter, such as a chemical compound, a metabolite, a nucleic acid, a polypeptide, a physical parameter (pH, temperature, viscosity, etc.), or a microorganism measured in a sample that has a decrease of at least 10%, preferably about 20%, more preferable about 40%, even more preferable about 50% and still more preferably a decrease of more than 75% when compared to the level, amount, or concentration of the same chemical compound, nucleic acid, polypeptide, physical parameter, or microorganism in a control sample. In some embodiments, the term describes the levels of NAD⁺. In some embodiments, the term describes a biological activity (e.g., inflammatory response). In some embodiments, the parameter is not detectable in a subject sample, while it is detectable in a control sample.

As used herein, the term “increased” and grammatical equivalents thereof refer to a level, amount, concentration of a parameter, such as a chemical compound, a metabolite, a nucleic acid, a polypeptide, a physical parameter (pH, temperature, viscosity, etc.), or a microorganism measured in a sample that has an increase of at least 30%, preferably about 50%, more preferable about 75%, and still more preferably an increase of more than 100% when compared to the level, amount, or concentration of the same chemical compound, nucleic acid, polypeptide, physical parameter, or microorganism in a control sample. In some embodiments, the term describes the levels of NAD⁺. In some embodiments, the parameter is detectable in a subject sample, while it is not detectable in a control sample.

The term “inhibit” includes the decrease, limitation, inactivation, prevention, or blockage, of, for example a particular action, function, or interaction. In some embodiments, PARP14 is “inhibited” if at least one biological or functional activity that is associated with PARP14 is terminated, slowed, block, or prevented (e.g., loss of NADase activity, ADP-ribose hydrolase, mono(ADP-ribosyl)transferase activity, and/or poly(ADP-ribosyl)transferase activity). In some embodiments, the term inhibits may refer to blocking the fall of NAD⁺ levels.

As used herein, the terms “treat,” “treating,” and “treatment” include: (1) preventing a pathological condition, disorder, or disease, i.e. causing the clinical symptoms of the pathological condition, disorder, or disease not to develop in a subject that may be predisposed to the pathological condition, disorder, or disease but does not yet experience any symptoms of the pathological condition, disorder, or disease; (2) inhibiting the pathological condition, disorder, or disease, i.e. arresting or reducing the development of the pathological condition, disorder, or disease or its clinical symptoms; or (3) relieving the pathological condition, disorder, or disease, i.e. causing regression of the pathological condition, disorder, or disease or its clinical symptoms. These terms encompass also prophylaxis, therapy, and cure. Treatment means any manner in which the symptoms of a pathological condition, disorder, or disease are ameliorated or otherwise beneficially altered. Preferably, the subject in need of such treatment is a mammal, more preferable a human.

A “variant”, “mutant” or “biologically inactive fragment” of a polypeptide (e.g., PARP14 as set forth in Table 1) refers to a polypeptide having the amino acid sequence of the polypeptide in which is altered in one or more amino acid residues. The variant may have “conservative” changes, wherein a substituted amino acid has similar structural or chemical properties (e.g., replacement of leucine with isoleucine). A variant may have “nonconservative” changes (e.g., replacement of glycine with tryptophan). For example, a variant or mutant may comprise any number of substitutions in Macro domain 1 as depicted in Table 2. Such mutants and variants are not functionally active (inactive) or lack biologically activity (e.g., NADase activity, ADP-ribose hydrolase, mono(ADP-ribosyl)transferase activity, and/or poly(ADP-ribosyl)transferase activity). Analogous variations may also include amino acid deletions or insertions, or both. Guidance in determining which amino acid residues may be substituted, inserted, or deleted to abolish biological or functional activity may be found using computer programs well known in the art, for example, LASERGENE software (DNASTAR).

The term “variant,” when used in the context of a polynucleotide sequence, may encompass a polynucleotide sequence related to that of a particular gene or the coding sequence thereof. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains, including functional and non-functional domains. Species variants are polynucleotide sequences that vary from one species to another. The resulting polypeptides generally will have significant amino acid identity relative to each other. A polymorphic variation is a variation in the polynucleotide sequence of a particular gene between individuals of a given species. Polymorphic variants also may encompass “single nucleotide polymorphisms” (SNPs) in which the polynucleotide sequence varies by one base.

### 1. Compositions of PARP14 Inhibitors and/or Mutants/Variants

### a. PARP14 Small Molecule Inhibitors

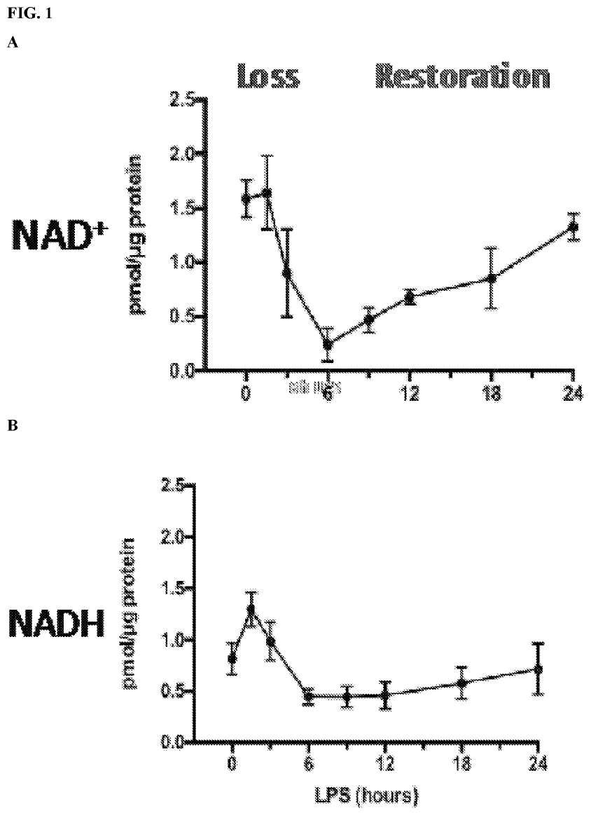

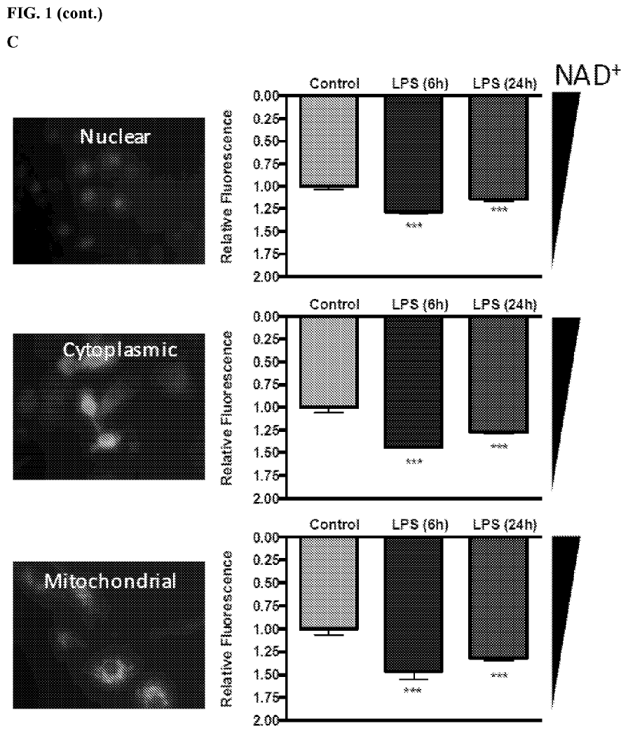

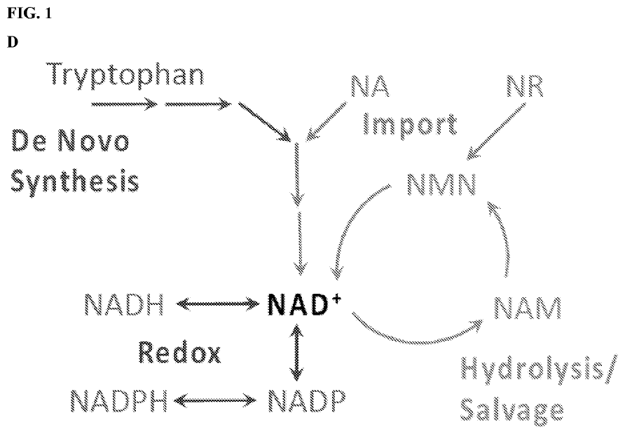

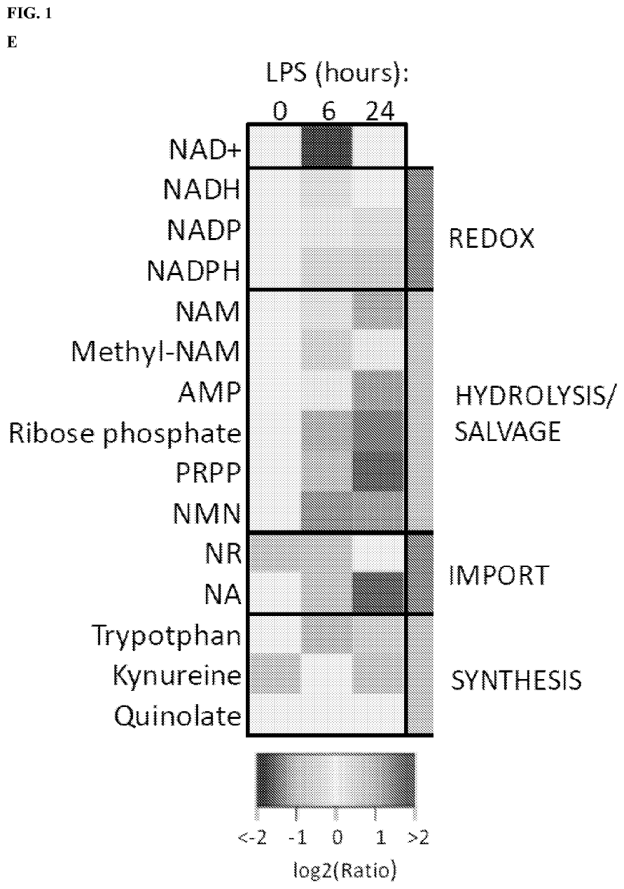

One aspect of the present invention relates to methods to block or prevent the fall of NAD⁺ levels using inhibitors that specifically target PARP14 (e.g., any of the sequences set forth in Table 1) to inhibit PARP14 activity (e.g., NADase activity, ADP-ribose hydrolase, mono(ADP-ribosyl)transferase activity, and/or poly(ADP-ribosyl)transferase activity). In certain embodiments, the PARP14 inhibitor, includes but not limited to, pan-PARP inhibitors, such as 3-aminobenzimide or PJ-34. In certain embodiments, the PARP14 inhibitors, include but not limited to, PARPs, 4, 10, 14, and 16 inhibitors, such as OUL35. Additional PARP4 inhibitors include PARP14 inhibitors of (Z)-4-(3-carbamoylphenylamino)-4-oxobut-2-enyl amides (e.g., compounds 4a-4v) as described in Upton et al. Bioorganic & Medicinal Chemistry Letters 27:2907-2911 (2017) (see Tables 1-3 of Upton). Additional pan-PARP14 inhibitors include 3-aminobenzamide, KU0058948, BGB-290, Olaparib, ABT-888, CEP-9722, DPQ, NU1025, EB-47, E7016, DiQ, DR2313, 4-ANI, ISQ, 3-hydroxybenzamide, CNQ, 3-AB, PJ34, DPQ, INH2BP, Iniparib, Niraparib (MK-4827), 6(5H)-phenanthridinone, 3-methyl-5-AIQ, Talazoparib, TIQ-A, XAV939, Veliparib, or Rucaparib. (See Wahlberg et al. (2012) Nature Biotechnology 30 (3): 283-289 and also FIG. 1 of Wahlberg).

In some embodiments, the PARP14 inhibitors directly suppress inflammasome activation. As chronic inflammasome activation may significantly contribute to increased inflammation leading to aging-related diseases (Goldberg et al. Immunological Reviews 265: 63-74 (2015); Poudel et al. J. of Leukocyte Biology 1-13 (2018); Hughes et al. Immunological Reviews 281:88-98 (2017)), such PARP14 inhibitors may be useful for treating or preventing aging or aging-related disorders, or disorders associate with inflammation.

In some embodiments, the inhibitor may specifically target and inhibit one or more Macro domains of PARP14. In some embodiments, the inhibitor targets and inhibits Macro domain 1 (e.g., Macro domain 1 as set forth in Table 2). Examples of Macro domain 1 inhibitors, include but are not limited to, NSC-61610 and NSC-127133. Additional Macro domain 1 inhibitors are described in Nguyen et al., J. Mol. Model., 20 (5):2216-1-2216-12 (2014) (see Table 1 of Ngyugen showing NCI-61610 (C₃₄H₂₄N₆O₂), NCI-25457 (C₂₄H₁₆N₂O), NCI-345647 a (C₃₀H₂₆O₁₀), NCI-670283 (C₂₅₄H₂₄O₂), and NCI_127133 (C₂₇H₁₈N₂O₄). In some embodiments, the inhibitor targets and inhibits Macro domains 2 and 3. Examples of Macro domain 2 inhibitors, include but are not limited to, GeA-69 as described in Schuller et al. ACS Chem Bio 12: 2866-2874 (2017). Examples of Macro domain 2 and 3 inhibitors, include but are not limited to, CBK004510 and CBK084521 as described in Ekblad et al. SLAS Discovery 1-10 (2018).

Assays to determine whether a certain inhibitor modulates PARP activity are known in the art. Examples of assays include a macrodomain-linked immunosorbent assay (MLISA) for mono-ADP-ribosyltransferases as described in Chen et al. Analytical Biochemistry 543:132-139 (2018).

### b. PARP14 Mutants/Variants

Another aspect of the present invention relates to methods to block the fall of NAD⁺ levels using PARP14 mutants and/or variants, that are not functionally active (inactive) and/or lack PARP14 activity (e.g., loss of NADase activity, ADP-ribose hydrolase, mono(ADP-ribosyl)transferase activity, and/or poly(ADP-ribosyl)transferase activity). Such PARP14 mutants and/or variants may mimic the biological effect of a PARP14 inhibitor (e.g., block the decline of NAD⁺ levels or increase NAD⁺ levels).

PARP14 mutants and/or variants include variations of 1, 2, 3, 4, 5, 6, 7, 8, 9, 10, 11, 12, 13, 14, 15, 16, 17, 18, 19, 20, or more amino acids on the 5′ end, on the 3′ end, or on both the 5′ and 3′ ends, of the sequences set forth in Table 1, resulting in an inactive and/or non-functional PARP14. In some embodiments, such a PARP14 mutant lacks PARP14 activity (e.g., NADase activity, ADP-ribose hydrolase, mono(ADP-ribosyl)transferase activity, and/or poly(ADP-ribosyl)transferase activity).

In some embodiments, PARP14 mutants and/or variants are polypeptide molecules comprising, consisting essentially of, or consisting of:
1) an amino acid sequence having at least 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 99.5% sequence identity across their full length with an amino acid sequence of SEQ ID NO: 1-14, or a biologically inactive fragment thereof;
2) an amino acid sequence having at least 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 99.5% sequence identity across their full length with an amino acid sequence of SEQ ID NO: 1-14, comprising at least one or more (e.g., one, two, three, four, five, six, seven, eight, nine, ten, or more) mutations in the Macro Domain 1 sequences as set forth in Table 2;
3) an amino acid sequence of SEQ ID NO: 1-14 having at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, or more amino acids, or any range in between, inclusive such as between 1400 and 1900 amino acids;
4) an amino acid sequence of SEQ ID NO: 1-14 having at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3500, 4000, 4500, 5000, 5500, 6000, 6500, 7000, 7500, 8000, 8500, 9000, 9500, 10000, or more amino acids, or any range in between, inclusive such as between 1400 and 1800 amino acids, comprising at least one or more (e.g., one, two, three, four, five, six, seven, eight, nine or ten) mutations in Macro Domain 1 as set forth in Table 2;
5) a biologically inactive fragment of an amino acid sequence of SEQ ID NO: 1-14 having at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2625, or more amino acids, or any range in between, inclusive such as between 1400 and 1900 amino acids;
6) a biologically inactive fragment of an amino acid sequence of SEQ ID NO: 1-14 having at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2625, or more amino acids, or any range in between, inclusive such as between 1400 and 1900 amino acids, comprising at least one or more (e.g., one, two, three, four, five, six, seven, eight, nine or ten) mutations in Macro Domain 1 as set forth in Table 1; or
7) nucleotide sequences encoding any of the polypeptides or amino acids set forth in 1) to 6) above.

In some embodiments, PARP14 mutants/variants may include deletion of, or mutation to, one or more amino acids, individually or combined, within the Macro domain 1 as noted in Table 1 or Table 2 (bolded, underlined, italicized, and highlighted amino acids). Such mutations may abolish PARP14 activity (e.g., NADase activity, ADP-ribose hydrolase, mono(ADP-ribosyl)transferase activity, and/or poly(ADP-ribosyl)transferase activity). Mutations may include any one of substitutions (conservative or non-conservative), insertions, or combinations thereof that may result in loss of NADase activity, ADP-ribose hydrolase, mono(ADP-ribosyl)transferase activity, and/or poly(ADP-ribosyl)transferase activity.

### c. Pharmaceutical Compositions

Inhibitors, nucleic acids, proteins, and other compounds described above can be administered to a subject according to methods known in the art. For example, nucleic acids encoding a protein or an antisense molecule can be administered to a subject as described above, e.g., using a viral vector such as retrovirus, lentivirus, or adeno-associated virus.

Pharmaceutical agents for use in accordance with the present methods may be formulated in conventional manner using one or more physiologically acceptable carriers or excipients. Thus, proteins and nucleic acids (e.g. PARP14 mutants/variants) described herein as well as compounds or agents (e.g., inhibitors of PARP14) and their physiologically acceptable salts and solvates may be formulated for into pharmaceutical agents for administration by, for example, injection, inhalation or insufflation (either through the mouth or the nose) or oral, buccal, parenteral or rectal administration. In some embodiment, the agent is administered locally, e.g., at the site where the target cells are present, such as by the use of a patch.

Compositions can increase the stress resistance of a mammalian cell e.g. stem cell in culture or in vivo, or a non-mammalian cell, e.g., a fish cell. Yeast cells include S. cerevisiae and C. albicans. The cell may also be a prokaryotic cell, e.g., a bacterial cell. The cell may also be a single-cell microorganism, e.g., a protozoan. The cell may also be a metazoan cell, a plant cell or an insect cell. The application of the methods described herein to a large number of cell types is based at least on the high conservation of PARP14 from humans to fungi, protozoans, metazoans and plants.

Pharmaceutical agents can be formulated for a variety of loads of administration, including systemic and topical or localized administration. Techniques and formulations generally may be found in Remington's Pharmaceutical Sciences, Meade Publishing Co., Easton, Pa. For systemic administration, injection is preferred, including intramuscular, intravenous, intraperitoneal, and subcutaneous. For injection, the agents can be formulated in liquid solutions, preferably in physiologically compatible buffers such as Hank's solution or Ringer's solution. In addition, the agents may be formulated in solid form and redissolved or suspended immediately prior to use. Lyophilized forms are also included.

For oral administration, the pharmaceutical compositions may take the form of, for example, tablets, lozenges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound. The pH of the formulations ranges from about 3 to about 11, but is ordinarily about 7 to 10.

Pharmaceutical agents that may oxidize and lose biological activity, especially in a liquid or semisolid form, may be prepared in a nitrogen atmosphere or sealed in a type of capsule and/or foil package that excludes oxygen.

For administration by inhalation, the agents may be conveniently delivered in the form of an aerosol spray presentation from pressurized packs or a nebuliser, with the use of a suitable propellant, e.g., dichlorodifiuoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin, for use in an inhaler or insufflator may be formulated containing a powder mix of the agent and a suitable powder base such as lactose or starch.

The pharmaceutical agents may be formulated for parenteral administration by injection, e.g., by bolus injection or continuous infusion. Formulations for injection may be presented in unit dosage form, e.g., in ampoules or in multi-dose containers, with an added preservative. The agents may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The pharmaceutical agents may also be formulated in rectal compositions such as suppositories or retention enemas, e.g., containing conventional suppository bases such as cocoa butter or other glycerides.

In addition to the formulations described previously, the agents may also be formulated as a depot preparation. Such long acting formulations may be administered by implantation (for example subcutaneously or intramuscularly) or by intramuscular injection. Thus, for example, the agents may be formulated with suitable polymeric or hydrophobic materials (for example as an emulsion in an acceptable oil) or ion exchange resins, or as sparingly soluble derivatives, for example, as a sparingly soluble salt. Controlled release formula also include patches, e.g., transdermal patches. Patches may be used with a sonic applicator that deploys ultrasound in a unique combination of waveforms to introduce drug molecules through the skin that normally could not be effectively delivered transdermally.

Pharmaceutical compositions (including cosmetic preparations) may comprise from about 0.00001 to 100% such as from 0.001 to 10% or from 0.1% to 5% by weight of one or more agents described herein.

In one embodiment, a pharmaceutical agent described herein, is incorporated into a topical formulation containing a topical earner that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

Pharmaceutical agents may be incorporated into ointments, which generally are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington's, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Exemplary water-soluble ointment bases are prepared from polyethylene glycols (PEGs) of varying molecular weight; again, reference may be had to Remington's, supra, for further information.

Pharmaceutical agents may be incorporated into lotions, which generally are preparations to be applied to the skin surface without friction, and are typically liquid or semiliquid preparations in which solid particles, including the active agent, are present in a water or alcohol base. Lotions are usually suspensions of solids, and may comprise a liquid oily emulsion of the oil-in-water type. Lotions are preferred formulations for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like. An exemplary lotion formulation for use in conjunction with the present method contains propylene glycol mixed with a hydrophilic petrolatum.

Pharmaceutical agents may be incorporated into creams, which generally are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington's, supra, is generally a nonionic, anionic, cationic or amphoteric surfactant.

Pharmaceutical agents may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of poly glycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

Pharmaceutical agents may be incorporated into gel formulations, which generally are semisolid systems consisting of either suspensions made up of small inorganic particles (two-phase systems) or large organic molecules distributed substantially uniformly throughout a carrier liquid (single phase gels). Single phase gels can be made, for example, by combining the active agent, a carrier liquid and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose and gelatin. Although gels commonly employ aqueous carrier liquid, alcohols and oils can be used as the carrier liquid as well.

Various additives, known to those skilled in the art, may be included in formulations, e.g., topical formulations. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients and preservatives. An optimum topical formulation comprises approximately: 2 wt. % to 60 wt. %, preferably 2 wt. % to 50 wt. %, solubilizer and/or skin permeation enhancer; 2 wt. % to 50 wt. %, preferably 2 wt. % to 20 wt. %, emulsifiers; 2 wt. % to 20 wt. % emollient; and 0.01 to 0.2 wt. % preservative, with the active agent and carrier (e.g., water) making of the remainder of the formulation. A skin permeation enhancer serves to facilitate passage of therapeutic levels of active agent to pass through a reasonably sized area of unbroken skin. Suitable enhancers are well known in the art and include, for example: lower alkanols such as methanol ethanol and 2-propanol; alkyl methyl sulfoxides such as dimethylsulfoxide (DMSO), decylmethylsulfoxide and tetradecylmethyl sulfoxide; pyrrolidones such as 2-pyrrolidone, N-methyl-2-pyrrolidone and N-(-hydroxyethyl)pyrrolidone; urea; N,N-diethyl-m-toluamide; C2-C6 alkanediols; miscellaneous solvents such as dimethyl formamide (DMF), N,N-dimethylacetamide (DMA) and tetrahydrofurfuryl alcohol; and the 1-substituted azacycloheptan-2-ones, particularly 1-n-dodecylcyclazacycloheptan-2-one (laurocapram; available under the trademark Azone® from Whitby Research Incorporated, Richmond, Va.).

Examples of solubilizers include, but are not limited to, the following: hydrophilic ethers such as diethylene glycol monoethyl ether (ethoxydiglycol) and diethylene glycol monoethyl ether oleate; polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides; alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol-2 (PPG-2) myristyl ether propionate, and the like.

Other active agents may also be included in formulations, e.g., anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation.

Topical skin treatment compositions can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507. Accordingly, also provided are closed containers containing a cosmetically acceptable composition.

In an alternative embodiment, a pharmaceutical formulation is provided for oral or parenteral administration, in which case the formulation may comprise an activating compound-containing microemulsion as described above, and may contain alternative pharmaceutically acceptable carriers, vehicles, additives, etc. particularly suited to oral or parenteral drug administration. Alternatively, an activating compound-containing microemulsion may be administered orally or parenterally substantially as described above, without modification.

Effective dose of a pharmaceutical agent depends at least on the nature of the condition being treated, toxicity, whether the compound is being used prophylactically (lower doses) or against an inflammatory disorder, the method of delivery, and the pharmaceutical formulation, and will be determined by the clinician using conventional dose escalation studies. It can be expected to be from about 0.0001 to about 100 mg/kg body weight per day; typically, from about 0.01 to about 10 mg/kg body weight per day; more typically, from about 0.01 to about 5 mg/kg body weight per day; most typically, from about 0.05 to about 0.5 mg/kg body weight per day. For example, the daily candidate dose for an adult human of approximately 70 kg body weight will range from 1 mg to 1000 mg, preferably between 5 mg and 500 mg, and may take the form of single or multiple doses.

Administration of an agent may be followed by measuring a factor in the subject, such as measuring the protein or transcript level of a gene (e.g., PARP14) described herein, or the level of NMN, NAD⁺, NADH, or nicotinamide. In an illustrative embodiment, a cell is obtained from a subject following administration of a pharmaceutical agent to the subject, such as by obtaining a biopsy, and the factor is determined in the biopsy. Alternatively, biomarkers, such as plasma biomarkers may be followed. The cell may be any cell of the subject, but in cases in which an agent is administered locally, the cell is preferably a cell that is located in the vicinity of the site of administration.

Other factors that may be monitored include a symptom of aging, weight, body mass, blood glucose sugar levels, blood lipid levels and any other factor that may be measured for monitoring diseases or conditions described herein.

### 2. Therapeutic Methods and Uses

Provided herein are methods of recovering from, treating, and preventing inflammation, cancer, aging, aging-related disorder, cell death, type II diabetes, radiation damage, radiation exposure, chemotherapy-induced damage, disorders associated with inflammation, cellular senescence, metabolic conditions, mitochondrial dysfunction, among others, improving DNA repair, cell proliferation, cell survival, mitochondrial biogenesis, among others, or increasing the life span of a cell or protect it against certain stresses or apoptosis, among others by providing a PARP14 inhibitor as described in Section 1 above (e.g., PARP14 small molecule inhibitor or PARP14 variant or mutant). Such methods include administering pharmaceutical agents that inhibit PARP14 (e.g., PARP14 small molecule inhibitors described in Section 1), or mimic the biological effect of PARP14 inhibition. In some embodiments, the methods involve providing pharmaceutical agents comprising PARP14 small molecule inhibitor, or PARP14 variant or mutant, that can block or prevent the fall, decline, or reduction in NAD⁺ levels. In some embodiments, the PARP14 inhibitors may act to increase the level or activity of nicotinamide dinucleotides (e.g., NAD⁺, NMN; NAD⁺ precursor pathways, such as a protein selected from the group consisting of NPT1, PNC1, NMA1 and NMA2; or NAD⁺ biosynthesis, such as enzymes selected from NMNAT-1, -2, and/or -3 or NAMPT). In some embodiments, the PARP14 mutants are not functionally active (inactive) and/or lack PARP14 activity (e.g., loss of NADase activity, ADP-ribose hydrolase, mono(ADP-ribosyl)transferase activity, and/or poly(ADP-ribosyl)transferase activity).

The protein level of any of the PARP14 mutants/variants described herein can be increased in a cell, e.g., by introducing into the cell a nucleic acid encoding the PARP14 mutant/variant protein operably linked to a transcriptional regulatory sequence directing the expression of the protein in the cell. Methods for expressing nucleic acids in cells and appropriate transcriptional regulatory elements for doing so are well known in the art. Alternatively, any of the PARP14 mutant/variant proteins described herein can be introduced into a cell, usually in the presence of a vector facilitating the entry of the protein into the cells, e.g., liposomes. Proteins can also be linked to transcytosis peptides for that purpose.

PARP14 mutants/variants that are biologically inactive or functionally defective can be identified according to methods known in the art and using an assay that can monitor the activity of the particular mutant or activity. Assays for determining the PARP12 inhibitor activity, or activity of any of the PARP14 mutants/variants set forth in Section 1 are described, e.g., in Chen et al. Analytical Biochemistry 543:132-139 (2018); Ekblad et al. SLAS Discovery 1-10 (2018); Schuller et al. ACS Chem Bio 12: 2866-2874 (2017); Upton et al. Bioorganic & Medicinal Chemistry Letters 27:2907-2911 (2017); Venkannagari et al. Cell Chemical Biology 23:1251-1260 (2016)). Alternatively, the PARP14 inhibitor, or activity of such a PARP14 mutant/variant, can be tested in an assay in which the life span of a cell is determined. For example, a cell is treated with a PARP14 inhibitor, or transfected with a nucleic acid comprising one or more copies of a sequence encoding a PARP14 mutant/variant protein or a control nucleic acid, and the life span of the cells is compared. A longer life span of a cell treated with a PARP14 inhibitor, or transfected with a portion of one of the PARP14 mutant/variant protein indicates that the PARP14 inhibitor or PARP14 mutant/variant protein is effective in increasing life span. Assays for determining the life span of a cell are known in the art. In particular, assays for determining the life span of a mammalian cell can be conducted as described, e.g., in Cell Growth, Differentiation and Senescence: A Practical Approach. George P. Studzinski (ed.). Instead of measuring the life span, one can also measure the resistance of a transfected cell to certain stresses, e.g., heatshock. Methods for measuring resistance to certain stresses are known in the art. In particular, assays for determining the resistance of a mammalian cell to heatshock can be conducted as described, e.g., in Bunelli et al. Exp. Cell Res. 262: 20 (1999).

Any means for the introduction of polynucleotides encoding PARP14 mutants/variants into mammals, human or non-human, or cells thereof may be adapted to the practice of this invention for the delivery of the various constructs into the intended recipient. In some, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. In some embodiments, the colloidal system is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139 (1995)). Formulation of DNA, e.g. with various lipid or liposome materials, may then be effected using known methods and materials and delivered to the recipient mammal. See, e.g., Canonico et al, Am J Respir Cell Mol Biol 10:24-29 (1994); Tsan et al, Am J Physiol 268; Alton et al., Nat Genet. 5:135-142 (1993) and U.S. Pat. No. 5,679,647 by Carson et al.

The expression of a PARP14 mutant/variant in cells of a subject to whom, e.g., a nucleic acid encoding the protein was administered, can be determined, e.g., by obtaining a sample of the cells of the patient and determining the level of the protein in the sample, relative to a control sample.

In another embodiment, a PARP14 mutant/variant is administered to the subject such that it reaches the target cells, and traverses the cellular membrane. Polypeptides can be synthesized in prokaryotes or eukaryotes or cells thereof and purified according to methods known in the art. For example, recombinant polypeptides can be synthesized in human cells, mouse cells, rat cells, insect cells, yeast cells, and plant cells. Polypeptides can also be synthesized in cell free extracts, e.g., reticulocyte lysates or wheat germ extracts. Purification of proteins can be done by various methods, e.g., chromatographic methods (see, e.g., Robert K Scopes Protein Purification: Principles and Practice Third Ed. Springer-Verlag, N.Y. 1994). In some embodiments, the polypeptide is produced as a fusion polypeptide comprising an epitope tag consisting of about six consecutive histidine residues. The fusion polypeptide can then be purified on a Ni⁺⁺ column. By inserting a protease site between the tag and the polypeptide, the tag can be removed after purification of the peptide on the Ni⁺⁺ column. These methods are well known in the art and commercial vectors and affinity matrices are commercially available.

Administration of polypeptides can be done by mixing them with liposomes, as described above. The surface of the liposomes can be modified by adding molecules that will target the liposome to the desired physiological location.

In some embodiment, a PARP14 mutant/variant is modified so that its rate of traversing the cellular membrane is increased. For example, the polypeptide can be fused to a second peptide which promotes “transcytosis,” e.g., uptake of the peptide by cells. In some embodiments, the peptide is a portion of the HIV transactivator (TAT) protein, such as the fragment corresponding to residues 37-62 or 48-60 of TAT, portions which are rapidly taken up by cell in vitro (Green et al. Cell 55:1179-1188 (1989)). In other embodiments, the internalizing peptide is derived from the Drosophila antennapedia protein, or homologs thereof. The 60 amino acid long homeodomain of the homeo-protein antennapedia has been demonstrated to translocate through biological membranes and can facilitate the translocation of heterologous polypeptides to which it is couples. Thus, polypeptides can be fused to a peptide consisting of about amino acids 42-58 of Drosophila antennapedia or shorter fragments for transcytosis. See for example Derossi et al. J Biol Chem 271:18188-18193 (1996); Derossi et al. J Biol Chem 269:10444-10450 (1994); and Perez et al. J Cell Sci 102:717-722 (1992).

In some embodiments, the introduction, treatment, or addition of a PARP14 inhibitor or PARP14 mutant/variant blocks the fall of NAD⁺ levels. In some embodiments, the introduction, treatment, or addition of a PARP14 inhibitor or PARP14 mutant/variant increases the levels of NAD⁺. The PARP14 inhibitor or PARP14 mutant may increase the level or activity of an enzyme involved in NAD⁺ biosynthesis, an enzymatically active fragment of such an enzyme, a nucleic acid encoding an enzyme involved in NAD⁺ biosynthesis, or an enzymatically active fragment of such a nucleic acid. Such enzymes may included mononucleotide adenylyl transferasel (NMNAT1), NMNAT2, NMNAT3, or nicotinamide phosphoribosyl transferase (NAMPT or NAMPRT). In some embodiments, the introduction, treatment, or addition of a PARP14 inhibitor or PARP14 mutant/variant suppresses inflammasome activation.

Another aspect of the invention provides a method for treating or preventing a disorder associated with inflammation. In some embodiments, the introduction, treatment, or addition of a PARP14 inhibitor or PARP14 mutant/variant may cause inflammation to decrease. In other embodiments, the inflammatory response is depressed or suppressed. A subject may self-administer the pharmaceutical agents (e.g., PARP14 inhibitor or PARP14 mutant/variant) as desired or a physician may administer the agents. Additionally a physician or other health care worker may select a delivery schedule. In some embodiments, the pharmaceutical agents (e.g., PARP14 inhibitor or PARP14 mutant/variant) are administered on a routine schedule. A routine refers to a predetermined designated period of time. The routine schedule may encompass periods of time which are identical or which differ in length, as long as the schedule is predetermined. For instance, the routine schedule may involve administration of the composition on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve, for example, administration of the pharmaceutical agents (e.g., PARP14 inhibitor or PARP14 mutant/variant) on a daily basis for the first week, followed by a monthly basis for several months, and then every three months after that. Any particular combination would be covered by the routine schedule as long as it is determined ahead of time that the appropriate schedule involves administration on a certain day. For use in therapy, an effective amount of the pharmaceutical agents (e.g., PARP14 inhibitor or PARP14 mutant/variant) can be administered to a subject by any mode. Administering a pharmaceutical composition of the present invention may be accomplished by any means known to the skilled artisan.

In some embodiments, the invention provides a method extending the life span of a cell, extending the proliferative capacity of a cell, slowing aging of a cell, promoting the survival of a cell, delaying cellular senescence in a cell, mimicking the effects of calorie restriction, increasing the resistance of a cell to stress, or preventing apoptosis of a cell, by contacting the cell with a pharmaceutical agent described herein (e.g. PARP14 inhibitor or PARP14 mutant/variant). In some embodiment, the methods comprise contacting the cell with a PARP14 inhibitor to thereby bind and modulate the activity of a biologically active PARP14, or fragment thereof, or a nucleic acid encoding same.

In another embodiment, a pharmaceutical agent described herein (e.g. PARP14 inhibitor or PARP14 mutant/variant) that increases the level of intracellular NAD⁺ may be used for recovering from, treating, or preventing a disease or condition induced or inflammation in a subject; methods for decreasing the inflammatory response in a subject; methods for recovering from, treating or preventing a disease or condition relating to life span (e.g., aging-related disorders); methods for recovering from, treating or preventing a disease or condition relating to the proliferative capacity of cells (e.g., cancer); and methods for recovering from, treating or preventing a disease or condition resulting from cell damage or death (e.g., DNA repair deficiency disorder). For example, the pharmaceutical agents disclosed herein can be used for recovery from mitigation, treatment, or amelioration of a DNA repair deficiency disorder. In certain embodiments, a method does not act by reducing the lethality caused by a disease, such as cancer.

In other embodiments, cells that are intended to be preserved for long periods of time are treated with inhibitors of PARP14 and/or PARP14 mutants/variants. The cells can be cells in suspension, e.g., blood cells, serum, biological growth media, or tissues or organs. For example, blood collected from an individual for administering to an individual can be treated as described herein, such as to preserve the blood cells for longer periods of time, such as for forensic purposes. Other cells that one may treat for extending their lifespan or protect against apoptosis include cells for consumption, e.g., cells from non-human mammals (such as meat), or plant cells (such as vegetables).

Generally, inhibitors of PARP14 may be used for extending the lifespan of a cell; extending the proliferative capacity of a cell; slowing aging of a cell; promoting the survival of a cell; delaying cellular senescence in a cell; or mimicking the effects of calorie restriction (see description below). In certain embodiments, a PARP14 inhibiting compound does not significantly increase the resistance of a cell to oxidative stress, although it may increase its resistance to other types of stresses. For example, a compound may increase the resistance of a cell to oxidative stress less than about 2, 5, 10, 30, or 100 fold relative to another compound, e.g., reservatrol.

In another embodiment, a pharmaceutical agents described herein (e.g. PARP14 inhibitor or PARP14 mutant/variant) may be used to treat cells useful for transplantation or cell therapy, including, for example, solid tissue grafts, organ transplants, cell suspensions, stem cells, bone marrow cells, etc. The cells or tissue may be an autograft, an allograft, a syngraft or a xenograft. The cells or tissue may be treated with the pharmaceutical agents described herein (e.g. PARP14 inhibitor or PARP14 mutant/variant) prior to administration/implantation, concurrently with administration/implantation, and/or post administration/implantation into a subject. The cells or tissue may be treated prior to removal of the cells from the donor individual, ex vivo after removal of the cells or tissue from the donor individual, or post implantation into the recipient. For example, the donor or recipient individual may be treated systemically with a pharmaceutical agents described herein (e.g. PARP14 inhibitor or PARP14 mutant/variant) or may have a subset of cells/tissue treated locally with a pharmaceutical agents described herein (e.g. PARP14 inhibitor or PARP14 mutant/variant). In certain embodiments, the cells or tissue (or donor/recipient individuals) may additionally be treated with another therapeutic agent useful for prolonging graft survival, such as, for example, an immunosuppressive agent, a cytokine, an angiogenic factor, etc.

In yet other embodiments, cells may be treated with a pharmaceutical agent described herein (e.g. PARP14 inhibitor or PARP14 mutant/variant) that increases the level of NAD⁺ in vivo, e.g., to increase their life span or prevent apoptosis. For example, skin can be protected from aging (e.g., developing wrinkles, loss of elasticity, etc.) by treating skin or epithelial cells with a pharmaceutical agent described herein (e.g. PARP14 inhibitors), or cream that increases the level intracellular NAD⁺. In some embodiments, skin is contacted with a cream, pharmaceutical or cosmetic composition comprising a pharmaceutical agent described herein (e.g. PARP14 inhibitor or PARP14 mutant/variant) that increases the level of intracellular NAD⁺. Examples of skin afflictions or skin conditions that may be treated in accordance with the methods described herein include disorders or diseases associated with or caused by inflammation, sun damage or natural aging. For example, the compositions described herein find utility for sunburn prevention, recovery from sunburn, and in the prevention or treatment of contact dermatitis (including irritant contact dermatitis and allergic contact dermatitis), atopic dermatitis (also known as allergic eczema), actinic keratosis, keratinization disorders (including eczema), epidermolysis bullosa diseases (including penfigus), exfoliative dermatitis, seborrheic dermatitis, erythemas (including erythema multiforme and erythema nodosum), damage caused by the sun or other light sources, discoid lupus erythematosus, dermatomyositis, psoriasis, skin cancer and the effects of natural aging. In another embodiment, a pharmaceutical agent described herein (e.g. PARP14 inhibitor or PARP14 mutant/variant) that increases the level of intracellular NAD⁺ may be used for the treatment of wounds and/or burns to promote healing, including, for example, first-, second- or third-degree burns and/or thermal, chemical or electrical burns. The formulations may be administered topically, to the skin or mucosal tissue, as an ointment, lotion, cream, microemulsion, gel, solution or the like, as further described herein, within the context of a dosing regimen effective to bring about the desired result.

In some embodiments, characteristics of aging can be obvious. For example, characteristics of older humans include skin wrinkling, graying of the hair, baldness, and cataracts, as well as hypermelanosis, osteoporosis, altered adiposity, cerebral cortical atrophy, lymphoid depletion, memory loss, thymic atrophy, increased incidence of diabetes type II, atherosclerosis, cancer, muscle loss, bone loss, and heart disease. Nehlin et al. Annals NY Acad Sci 980: 176-79 (2000). Other aspects of mammalian aging include weight loss, lordokyphosis (hunchback spine), absence of vigor, lymphoid atrophy, decreased bone density, dermal thickening and subcutaneous adipose tissue, decreased ability to tolerate stress (including heat or cold, wounding, anesthesia, and hematopoietic precursor cell ablation), liver pathology, atrophy of intestinal villi, skin ulceration, amyloid deposits, and joint diseases. Tyner et al. Nature 415:45-53 (2002).

Careful observation reveals characteristics of aging in other eukaryotes, including invertebrates. For example, characteristics of aging in the model organism C. elegans include slow movement, flaccidity, yolk accumulation, intestinal autofluorescence (lipofuscin), loss of ability to eat food or dispel waste, necrotic cavities in tissues, and germ cell appearance.

Those skilled in the art will recognize that the aging process is also manifested at the cellular level. Cellular aging is manifested in reduced mitochondrial function, loss of doubling capacity, increased levels of apoptosis, changes in differentiated phenotype, and changes in metabolism, e.g., decreased fatty acid oxidation, respiration, and protein synthesis and turnover.

Given the programmed nature of cellular and organismal aging, it is possible to evaluate the “biological age” of a cell or organism by means of phenotypic characteristics that are correlated with aging. For example, biological age can be deduced from patterns of gene expression, resistance to stress (e.g., oxidative or genotoxic stress), rate of cellular proliferation, and the metabolic characteristics of cells (e.g., rates of protein synthesis and turnover, mitochondrial function, ubiquinone biosynthesis, cholesterol biosynthesis, ATP levels within the cell, levels of a Krebs cycle intermediate in the cell, glucose metabolism, nucleic acid metabolism, ribosomal translation rates, etc.). As used herein, “biological age” is a measure of the age of a cell or organism based upon the molecular characteristics of the cell or organism. Biological age is distinct from “temporal age,” which refers to the age of a cell or organism as measured by days, months, and years.

The rate of aging of an organism, e.g., an invertebrate (e.g., a worm or a fly) or a vertebrate (e.g., a rodent, e.g., a mouse) can be determined by a variety of methods, e.g., by one or more of: a) assessing the life span of the cell or the organism; (b) assessing the presence or abundance of a gene transcript or gene product in the cell or organism that has a biological age-dependent expression pattern; (c) evaluating resistance of the cell or organism to stress, e.g., genotoxic stress (e.g., etopocide, UV irradiation, exposure to a mutagen, and so forth) or oxidative stress; (d) evaluating one or more metabolic parameters of the cell or organism; (e) evaluating the proliferative capacity of the cell or a set of cells present in the organism; and (f) evaluating physical appearance or behavior of the cell or organism. In one example, evaluating the rate of aging includes directly measuring the average life span of a group of animals (e.g., a group of genetically matched animals) and comparing the resulting average to the average life span of a control group of animals (e.g., a group of animals that did not receive the test compound but are genetically matched to the group of animals that did receive the test compound). Alternatively, the rate of aging of an organism can be determined by measuring an aging-related parameter.

The pharmaceutical agents described herein (e.g. PARP14 inhibitor or PARP14 mutant/variant) that increases the level of intracellular NAD⁺ can also be administered to subjects for treatment of diseases, e.g., chronic diseases, associated with cell death, in order to protect the cells from cell death. Exemplary diseases include those associated with neural cell death, neuronal dysfunction, or muscular cell death or dysfunction, such as Parkinson's disease, Alzheimer's disease, multiple sclerosis, amyotropic lateral sclerosis, and muscular dystrophy; AIDS; fulminant hepatitis; diseases linked to degeneration of the brain, such as Creutzfeld-Jakob disease, retinitis pigmentosa and cerebellar degeneration; myelodysplasis such as aplastic anemia; ischemic diseases such as myocardial infarction and stroke; hepatic diseases such as alcoholic hepatitis, hepatitis B and hepatitis C; joint-diseases such as osteoarthritis; atherosclerosis; alopecia; damage to the skin due to UV light; lichen planus; atrophy of the skin; cataract; and graft rejections. Cell death can also be caused by surgery, drug therapy, chemical exposure, or radiation exposure.

The pharmaceutical agents described herein (e.g. PARP14 inhibitor or PARP14 mutant/variant) can also be administered to a subject suffering from an acute damage to an organ or tissue, e.g., a subject suffering from stroke or myocardial infarction or a subject suffering from a spinal cord injury or used to repair an alcoholic's liver.

Subjects that may be treated as described herein include eukaryotes, such as mammals, e.g., humans, ovines, bovines, equines, porcines, canines, felines, non-human primate, mice, and rats. Cells that may be treated include eukaryotic cells, e.g., from a subject described above, or plant cells, yeast cells and prokaryotic cells, e.g., bacterial cells.

In some embodiments, a composition can be taken by subjects as a food or dietary supplement. In some embodiments, such a composition is a component of a multi-vitamin complex or as a multi-drug regimen. Compositions can also be added to existing formulations that are taken on a daily basis, e.g., statins and aspirin. Compositions may also be used as food additives. In some embodiments, the multi-drug complex or regimen would include drugs or compositions for the treatment or prevention of aging-related diseases, e.g., stroke, heart disease, arthritis, high blood pressure, Alzheimer's. In some embodiments, this multi-drug regimen would include chemotherapeutic drugs for the treatment of cancer. In some embodiments, a composition could be used to protect non-cancerous cells from the effects of chemotherapy or for recovering from, treating, or preventing chemotherapy-induced damage.

The pharmaceutical agents described herein (e.g. PARP14 inhibitor or PARP14 mutant/variant) may also be applied during developmental and growth phases in mammals, plants, insects or microorganisms, in order to, e.g., alter, retard or accelerate the developmental and/or growth process.

In other embodiments, cells obtained from a subject, e.g., a human or other mammal, are treated according to methods described herein and then administered to the same or a different subject. Accordingly, cells or tissues obtained from a donor for use as a graft can be treated as described herein prior to administering to the recipient of the graft. For example, bone marrow cells can be obtained from a subject, treated ex vivo, e.g., to extend their lifespan, and then administered to a recipient. The graft can be an organ, a tissue or loose cells.

In yet other embodiments, cells are treated in vivo, e.g., to increase their lifespan or prevent apoptosis. For example, skin can be protected from aging, e.g., developing wrinkles, by treating skin, e.g., epithelial cells, as described herein.

Topical formulations described above may also be used as preventive, e.g., chemopreventive, compositions. When used in a chemopreventive method, susceptible skin is treated prior to any visible condition in a particular individual.

In one embodiment, cells are treated in vitro to mimic caloric restriction, such as to extend their lifespan, e.g., to keep them proliferating longer and/or increasing their resistance to stress or prevent apoptosis.

Compounds can also be delivered locally, e.g., to a tissue or organ within a subject, such as by injection, e.g., to extend the lifespan of the cells; protect against apoptosis or induce apoptosis.

Generally, PARP14-inhibiting compounds may be used in methods for treating or preventing a disease or condition induced or exacerbated by cellular senescence in a subject; methods for decreasing the rate of senescence of a subject, e.g., after onset of senescence; methods for extending the lifespan of a subject; methods for treating or preventing a disease or condition relating to lifespan; methods for treating or preventing a disease or condition relating to the proliferative capacity of cells; and methods for treating or preventing a disease or condition resulting from cell damage or death. In certain embodiments, the disease or condition does not result from oxidative stress. In certain embodiments, a method does not significantly increase the resistance of the subject to oxidative stress. In certain embodiments, the method does not act by decreasing the rate of occurrence of diseases that shorten the lifespan of a subject. In certain embodiments, a method does not act by reducing the lethality caused by a disease, such as cancer.

Other combination therapies include conjoint administration with nicotinamide, NAD⁺ or salts thereof, or other Vitamin B3 analogs. Carnitines, such as L-carnitine, may also be co-administered, particularly for recovering from, treating, or preventing cerebral stroke, loss of memory, pre-senile dementia, Alzheimer's disease or preventing or treating disorders elicited by the use of neurotoxic drugs. Cyclooxygenase inhibitors, e.g., a COX-2 inhibitor, may also be co-administered for recovering from, treating, or preventing certain conditions described herein, such as an inflammatory condition or a neurologic disease.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

As can be appreciated from the disclosure above, the present invention has a wide variety of applications. The invention is further illustrated by the following examples, which are only illustrative and are not intended to limit the definition and scope of the invention in any way.

### EXEMPLIFICATIONS

### Example 1

### NAD⁺ Levels Fluctuate in Response to LPS

NAD⁺ levels rapidly decline upon LPS-activation in bone marrow derived macrophages (BMDMs). NADH levels do not increase, suggesting that NAD⁺ is hydrolyzed rather than reduced. NAD⁺ and NADH levels recover after about 24 hours (FIG. 1A-1B). NAD⁺ and NADH levels do not change during anti-inflammatory IL-4 stimulation, suggesting that this phenomenon is a pro-inflammatory response. These data were generated with an enzyme-coupled luciferase assay. Using a genetically-encoded fluorescent reporter in RAW264.7 immortalized macrophages (FIG. 1C), it was confirmed that these changes occur in all three major compartments of the cell. Mass spectrometry-based metabolomics in BMDMs were performed which confirmed that an increase in NAD⁺ hydrolysis is driving the phenomenon (FIG. 1D-1E). Additional experiments using flux metabolomics in collaboration with Professor Marie Migaud at the University of South Alabama may be used in order to determine the rates of consumption and salvage of NAD⁺, before and after treatment with LPS. In such experiments, isotopically-labeled NMN (an NAD⁺ precursor) are synthesized with labels on the phosphate, ribose, and nicotinamide groups, which allows tracing of multiple downstream metabolites.

As NAD⁺ levels are important for many cellular functions, including energy metabolism, DNA repair, and epigenetic maintenance, these findings indicate that a rapid consumption of NAD⁺ is likely an important aspect of the inflammatory response.

### Example 2

### PARP14 is Necessary for LPS-Induced NAD⁺ Destruction

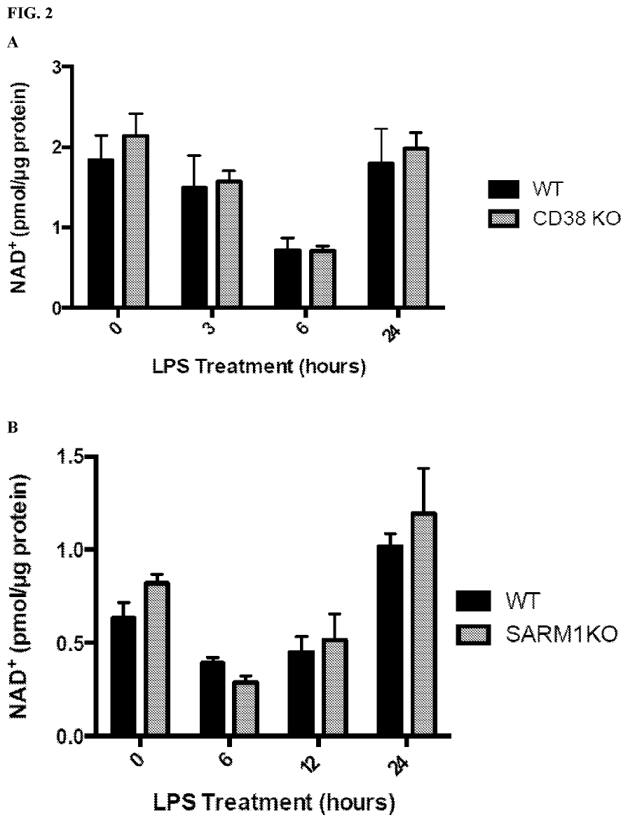

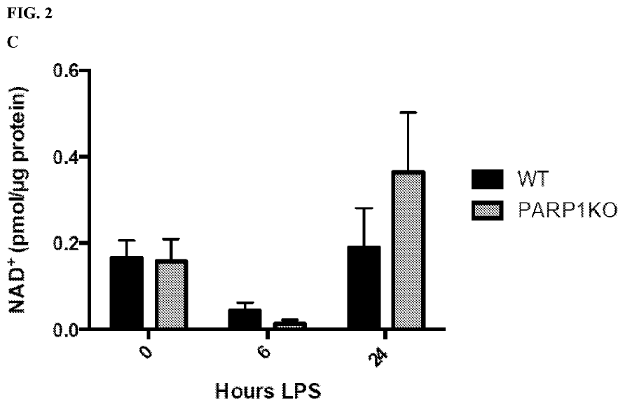

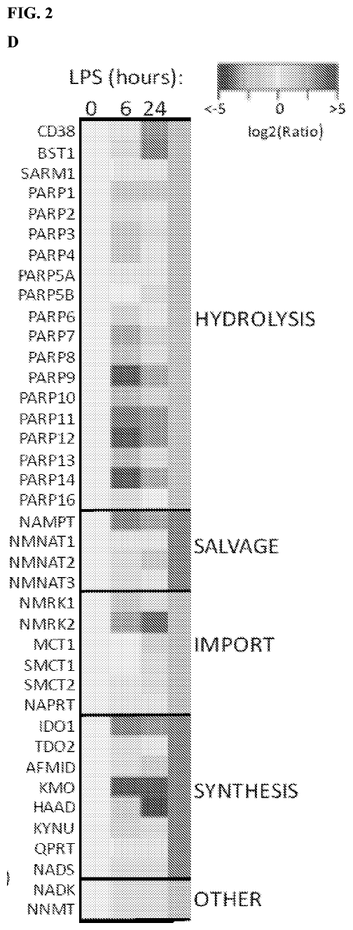

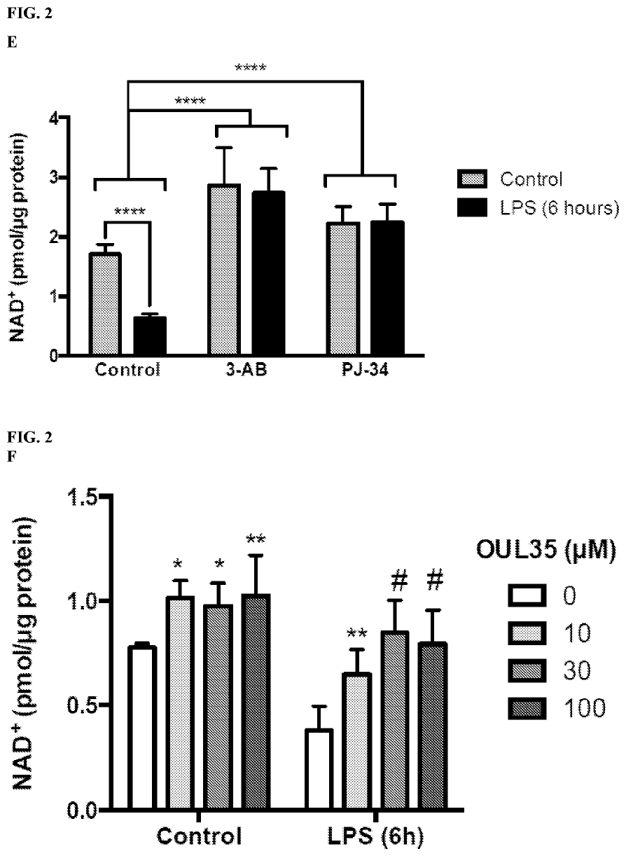

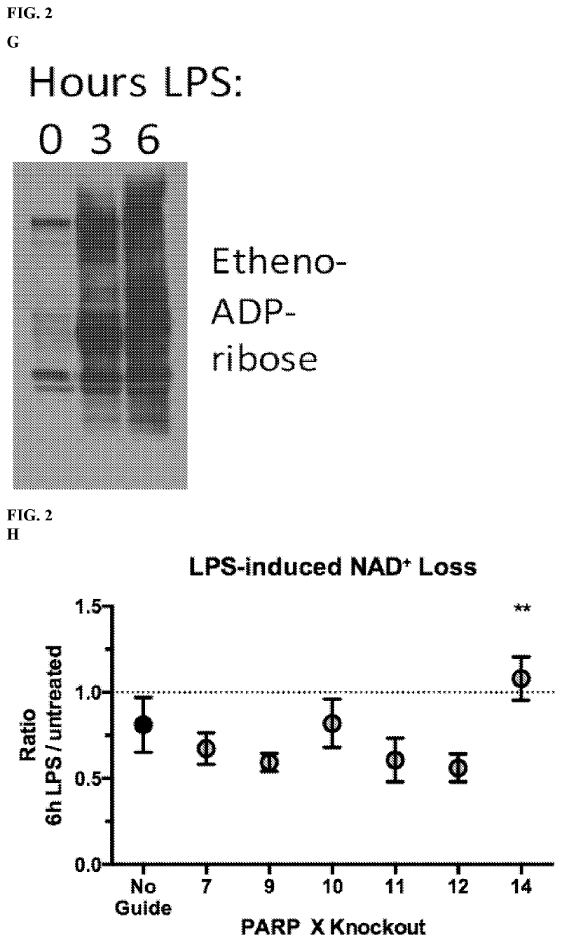

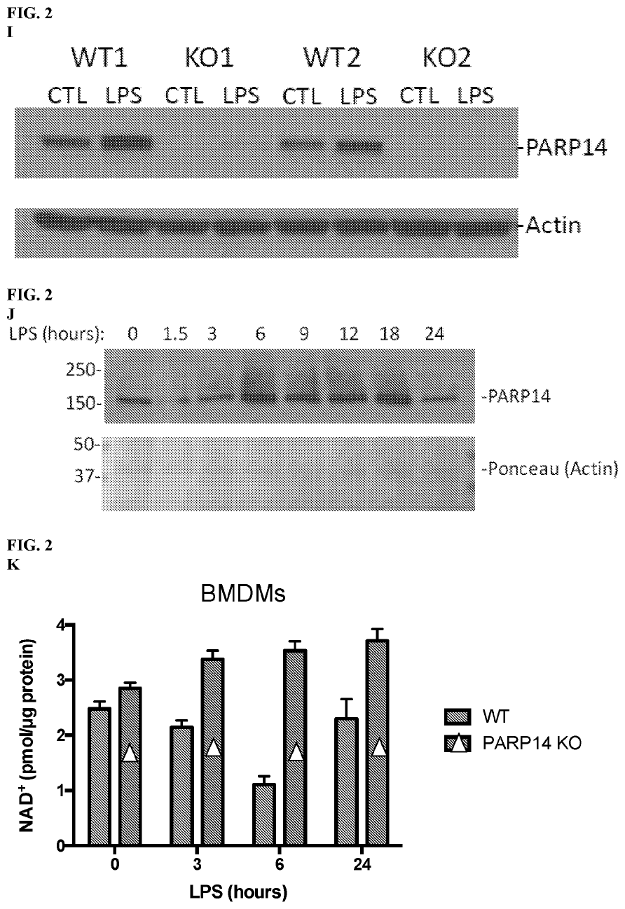

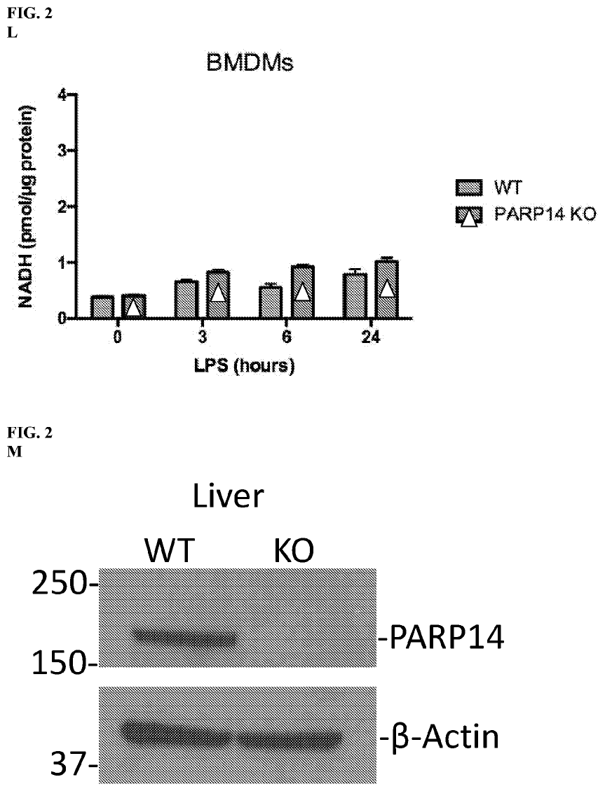

Prior studies had ruled out CD38, SARM1, and PARP1 as causes for the NAD⁺-decline (FIG. 2A-2C). To identify new candidates, RNA levels of relevant enzymes were characterized at 0, 6, and 24 hours post-LPS treatment (FIG. 2D, 2J). Interestingly, many members of the PARP family displayed an inverse relationship to NAD⁺ levels, increasing at 6 hours, and falling again at 24 hours. PARPs are enzymes that hydrolyze NAD⁺ in order to transfer ADP-ribose to target proteins. In support that a member of the PARP family is responsible, two pan-PARP inhibitors, 3-aminobenzimide and PJ-34, completely blocked the LPS-induced drop in NAD⁺ levels (FIG. 2E). Additionally, OUL35 (Venkannagari et al. Cell Chemical Biology 23:1251-1260 (2016)), a more specific inhibitor of PARPs 4, 10, 14, and 16, but importantly with no activity against PARP1, also blocked the drop (FIG. 2F). While poly-ADP-ribose levels fall with LPS-stimulation, I have observed an overall increase in ADP-ribosylation activity, further suggesting that members of the PARP family could be rapidly hydrolyzing NAD⁺ (FIG. 2G).

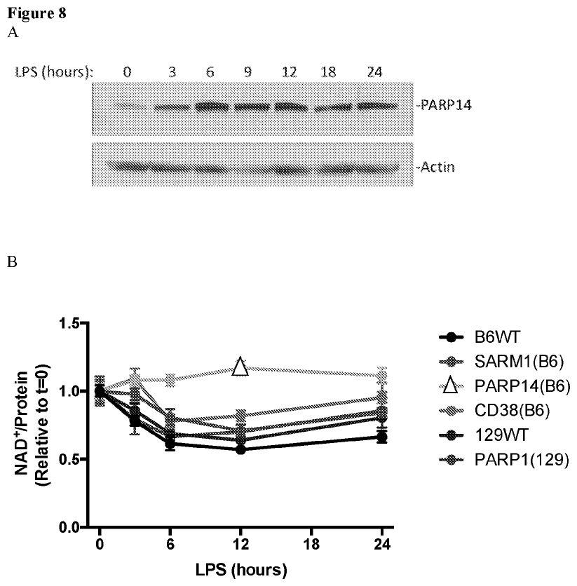

To determine if a single PARP enzyme was responsible, the top 6 PARPs whose RNA increased the most at 6 hours LPS-stimulation were identified, and knockout cell lines were generated in RAW264.7 immortalized macrophages using CRISPR/Cas9. Cell lines were screened by PCR and DNA sequencing, and four knockout lines were selected for each PARP. When stimulated with LPS for 6-hours, NAD⁺ levels fell in all cell lines except for the PARP14 KOs (FIGS. 2H and 2I). In BMDMs, PARP14 protein levels correlate with RNA levels (FIG. 8A). BMDMs deficient in other NADases such as SARM1, CD38, and PARP1, display the NAD⁺ fluctuation seen in WT cells, while PARP14 KO cells do not (FIG. 8G).

These findings indicate that if PARP14 is indeed necessary, it would represent an undescribed major regulator of NAD⁺ levels.

Additional experiments to confirm the observation in PARP14 KO BMDMs may be obtained using heterozygous PARP14 breeder mice from the lab of Professor Masanori Aikawa, at Brigham and Women's Hospital. BMDMs from WT and KO littermates can be generated and tested for their response to LPS on NAD⁺ levels. Additional experiments may be conducted to determine the optimal time course of PARP14 protein levels and of ADP-riboslyation levels/activity.

### Example 3

### PARP14 is Sufficient for the NAD⁺ Decline

In order to determine whether PARP14 acts alone to hydrolyze NAD⁺, or if it requires a binding partner or a post-translational modification in order to activate it, the coding sequence of mouse PARP14 was cloned into a lentiviral plasmid. BMDMs may be transduced with PARP14 lentivirus to overexpress the protein in the absence of LPS, then NAD⁺ levels may be measured. The PARP14 may be cloned into a lentiviral vector with an inducible promoter, then a similar experiment may be performed with a short-term induction of PARP14.

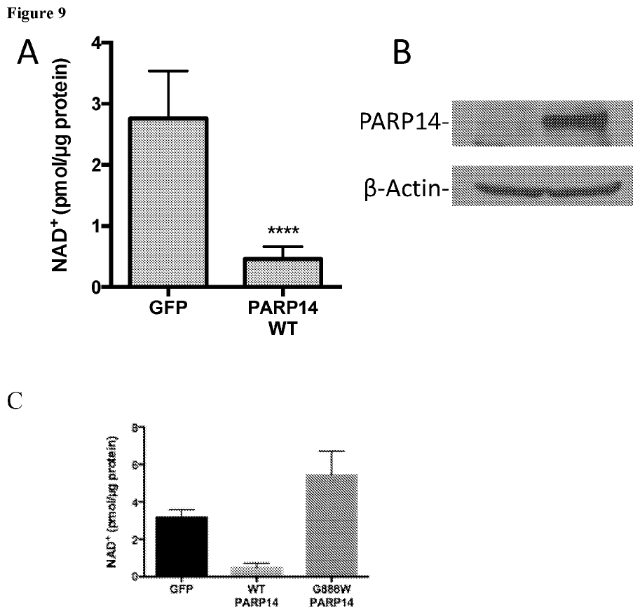

Overexpression of PARP14 in BMDMs delivered by lentivirus slightly lowered NAD⁺ levels. Massive overexpression of PARP14 in HEK-293T cells was achieved, which do not appear to normally express the enzyme. This results in a large decrease in intracellular NAD⁺ levels (FIG. 9A-B).

### Example 4

### Mechanism of PARP14 NADase Activity

GST-tagged PARP14 was overexpressed and immunoprecipitated in E. coli. When such recombinant protein was added to an in vitro NADase activity assay, modest activity was observed relative to a CD38 positive control. There was a clear increase fluorescence (representing NAD-hydrolysis) over a 48-hour period. For confirmation, additional controls (such as inclusion of inhibitors) and experiments may be needed (FIG. 3A).

PARP14 may be a highly active NADase. PARP14 may rapidly mono- or poly-ADP-ribosylate other proteins, to such a rate as to lower intracellular NAD⁺ levels. In vitro, PARP14 appears to be able to ADP-ribosylate itself (FIG. 3B).

A more compelling hypothesis is informed by an analysis of PARP14's annotated domains. In addition to its C-terminal PARP domain, it contains three Macro domains (FIG. 3C). Macro domains are ADP-ribose binding (as in the case of histone macro-H2A), and in some cases, they may also hydrolyze ADP-ribose from ADP-ribosylated proteins (as in the case of the enzyme MacroD1). An amino acid alignment suggests that while PARP14′s second and third Macro domains are merely ADP-ribose-binding, its first Macro domain likely has ADP-ribose hydrolase activity (FIG. 3D). This suggests a two-step mechanism for progressively hydrolyzing NAD⁺: 1) PARP14 consumes an NAD⁺ molecule via its PARP domain to ADP-riboslyate itself at some distal site; then 2) its Macro domain removes this ADP-ribose, freeing up this distal site to accept another ADP-ribose. If this second hypothesis is correct, inhibiting the Macro domain would be as effective as a PARP inhibitor in blocking the fall of NAD⁺ levels. While no Macro domain inhibitors have been discovered, a number of publically available molecules were recently computationally predicted as such (Nguyen et al., J. Mol. Model., 20 (5):2216-1-2216-12 (2014)). Six such predicted inhibitors were tested. Two predicted inhibitors, NSC-61610 and NSC-127133, were able to block the fall in NAD⁺ levels (FIGS. 3E and 3F). NSC-61610 was recently described as anti-inflammatory in a model of influenza, albeit via a different mechanism (Leber et al., Front. Immunol., 8 (178)1-14 (2017)).

If the first hypothesis is true, PARP14 may be a highly-active ADP-riboslytransferase on par with the activity of activated PARP1. If the second hypothesis is true, PARP14 has evolved a fascinating mechanism where two domains with antagonistic activity cooperate to consume a metabolite.

To further investigate the mechanism, robust PARP14 NADase activity may be examined in vitro. If recombinant PARP14 does not show such activity, endogenous PARP14 may be immunoprecipitated from LPS-treated BMDMs and added to the assay. If higher NADase activity is observed, post-translational modifications may be identified and mimicked with PARP14 mutants, which can be expressed. Additional experiments may included selectively mutating the PARP and Macro domains and using both an in vitro NADase assay (e.g., FIG. 3A), and ADP-ribosylation assay (e.g., FIG. 3B).

### Example 5

### The Effects of Decreased NAD⁺ Levels on Secretion of Cytokines

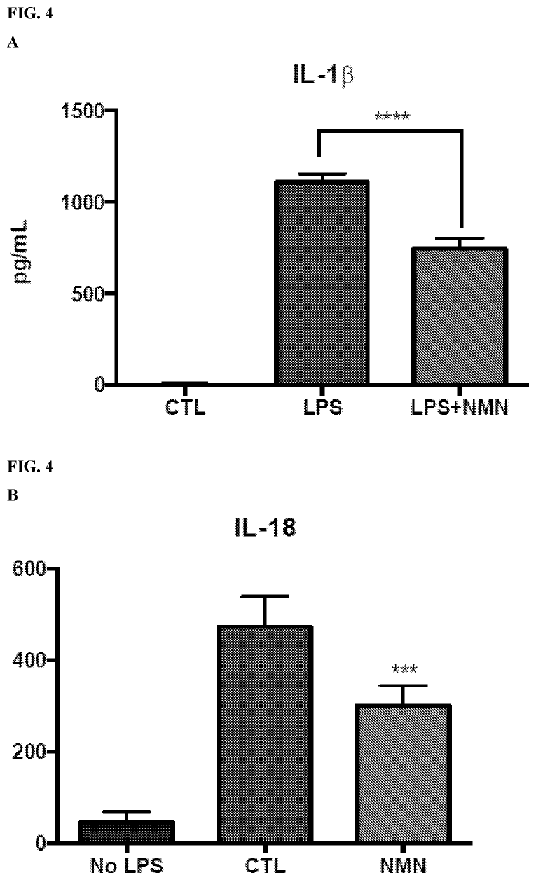

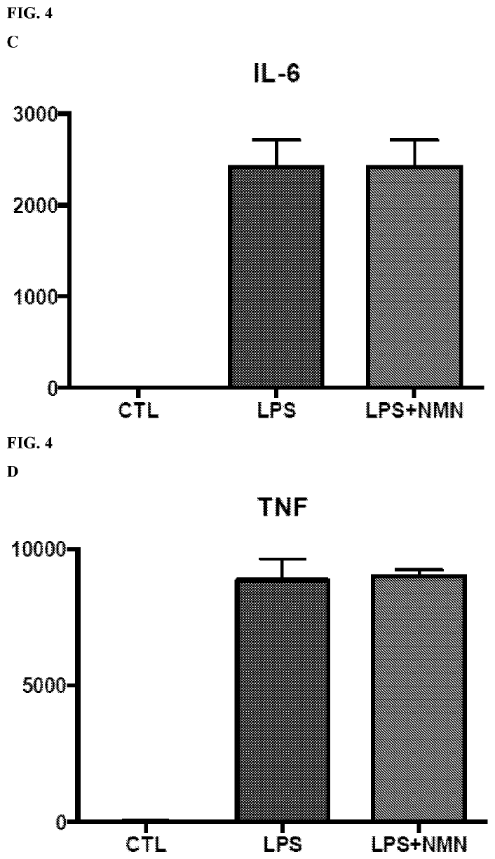

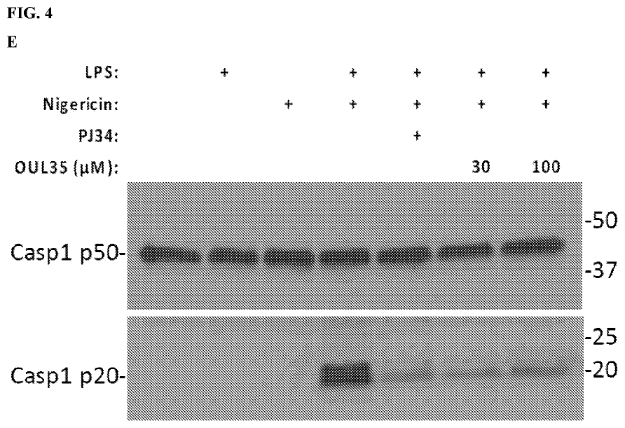

Previous studies showed that NMN, which raises levels of NAD⁺, can suppress secretion of IL-1β and IL-18, but not of IL-6 or TNF (FIG. 4A-4D). IL-1β and IL-18 are unique cytokines in that they require activation of an inflammasome to be processed for release. Interestingly, the NLRP3 inflammasome is responsive to signals of metabolic distress.

As 3-AB, PJ-34, OUL35, NSC-61610 and NSC-127133 can raise NAD⁺ levels in activated macrophages beyond NMN, cells may be treated with these compounds, and the media will be sent to Eve Technologies for a multiplex cytokine array. Other aspects of macrophage function may be characterized, such as reactive oxygen species generation, glucose uptake, and phagocytosis ability.

### Example 6

### PARP14's Effect on Inflammasome Activation

One hallmark of inflammasome activation is cleavage of caspase-1 to generate a 20 kD peptide. PJ-34 and OUL35 were shown to both suppress this (FIG. 4E).

These PARP inhibitors are directly suppressing inflammasome activation. Additional experiments to show inflammasome activation may be tested with PJ-34 and OUL35. These experiments may include IL-1β and IL-18 secretion (ELISA), IL-1β and IL-18 cleavage (Western), and ASC oligomerization (immunofluorescence). The same series of experiments may be performed on PARP14KO BMDMs.

### Example 7

### The Efficacy of PARP14 Inhibitors for Suppressing Inflammation In Vivo

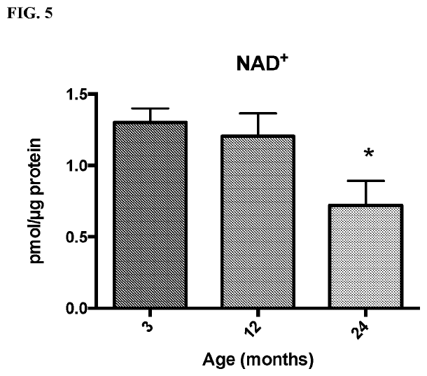

NAD⁺ levels dropped in peritoneal macrophages during aging (FIG. 5). It is likely that increased PARP levels/activity and falling NAD⁺ is a driver of aging and inflammatory disease. These findings contradict published data showing that PAR14KO mice as having more severe inflammation in models of coronary artery disease (Iwata et al., Nat. Commun., 7 (12849):1-19 (2016)) and allergy (Krishnamurthy et al., Immunology 152 (3):451-461 (2017)). However, PARP14 levels do not appear to change significantly during aging in liver or spleen.

To further investigate whether PARP14 may be a driver of inflammatory diseases and aging, OUL35 may be tested in animal studies. Additional experiments include testing OUL35 in a zebrafish larva model of infection in collaboration with the lab of Professor Deborah Hung (MGH). If a post-translational modification or binding partner is required for PARP14 activation, these will be probed for during aging and disease

Multiple doses of OUL35 may be tested in mice to determine toxicity and effect on any raise NAD⁺ levels. Upon choosing a dose, OUL35 may be tested in a collagen-induced arthritis model, which is highly dependent on the cytokine IL-1β. Other models of inflammatory disease (such as sepsis) and aging can also be tested.

### Example 8

### NAD⁺ Levels Fall After TLR Activation

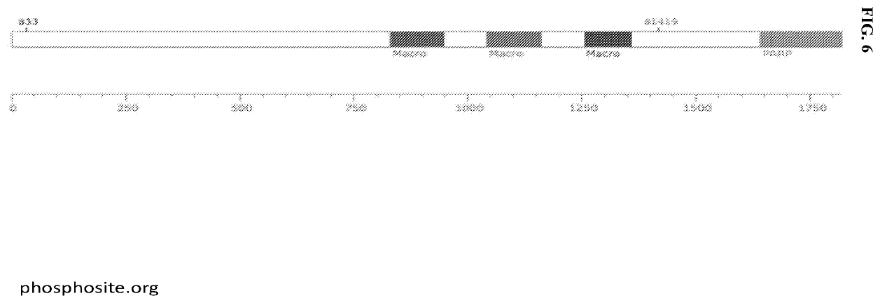

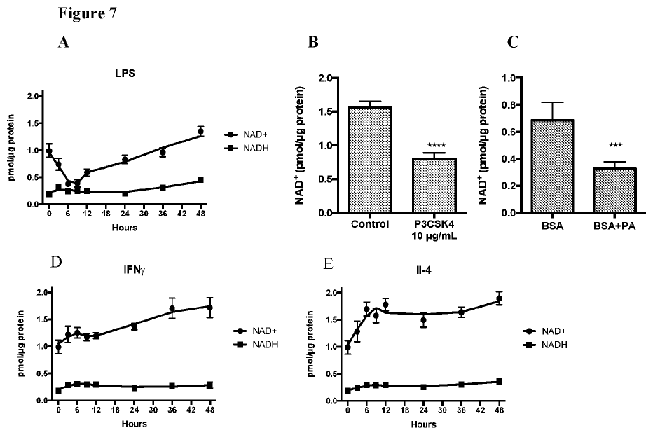

NAD⁺ levels rapidly decline upon LPS-activation in bone marrow derived macrophages (BMDMs). NADH levels do not increase, suggesting that NAD⁺ is hydrolyzed rather than reduced. NAD⁺ and NADH levels recover after about 24 hours (FIG. 7A). NAD⁺ levels also fall in response to other Toll-like receptor (TLR) activators such as P3SCK4 and palmitic acid (PA) (FIG. 7B-C).

### Example 9

### NRH is a Potent NAD-Raising Precursor

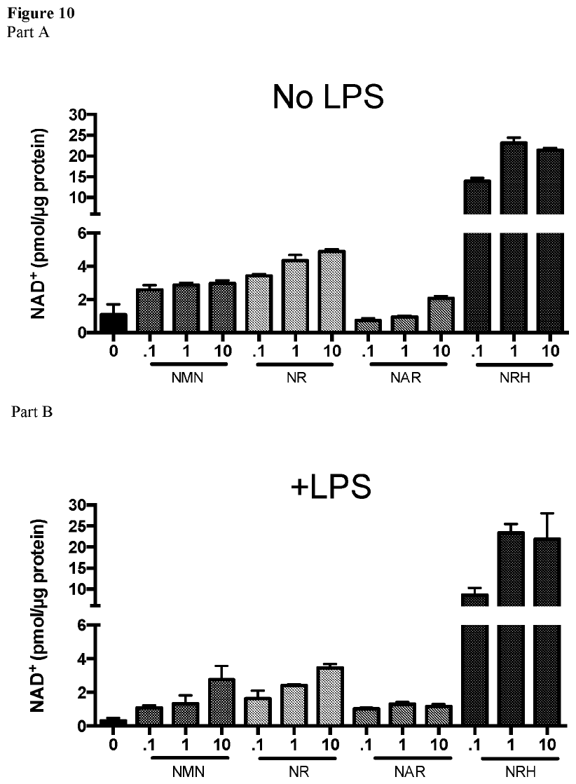

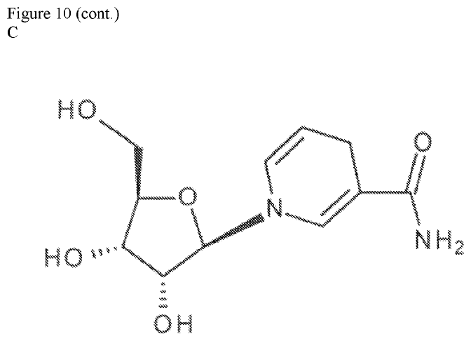

Nicotinamide mononucleotide (NMN) could slightly protect NAD⁺ levels in macrophages exposed to LPS, but only at very high concentrations, >1M. Nicotinamide riboside hydride (NRH), the reduced form of NR, is extremely potent at raising NAD⁺ levels in BMDMs (FIG. 10A-C). As a non-natural metabolite, it may bypass hydrolyzing enzymes or regulatory feedback mechanisms. NRH can be used to protect NAD⁺ levels in the presence of LPS, at pharmacological concentrations.

### Example 10

### Characterize the Effects of Decreased NAD⁺ Macrophage Function

NMN, which raises levels of NAD⁺, can suppress secretion of IL-1β and IL-18. PARPi and NRH more potent protectors of NAD⁺ levels (FIG. 11A). The most robust observation seen is a restoration of poly-ADP-ribose (PAR) levels (FIG. 11B). While this is clearly an effect of restoring NAD⁺ levels, its significance on macrophage function is unclear. It has been observed that suppression of inflammasome activation as measured by Caspase 1 cleavage, required for IL-1β and IL-18 secretion (FIG. 11C), however this is only somewhat reproducible, working sometimes and not other times. Inflammatory gene expression may be altered, but this must be repeated with an increased N-value (FIG. 11D-F).

### Example 11

### Characterize the Effects of Decreased NAD⁺ Macrophage Metabolism

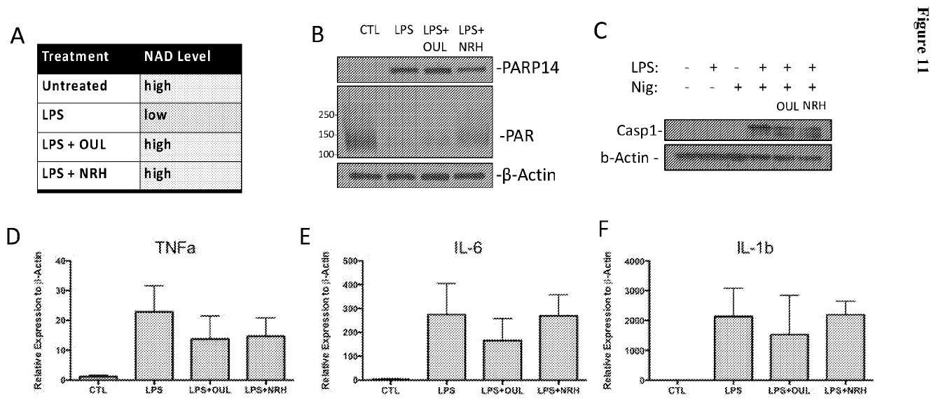

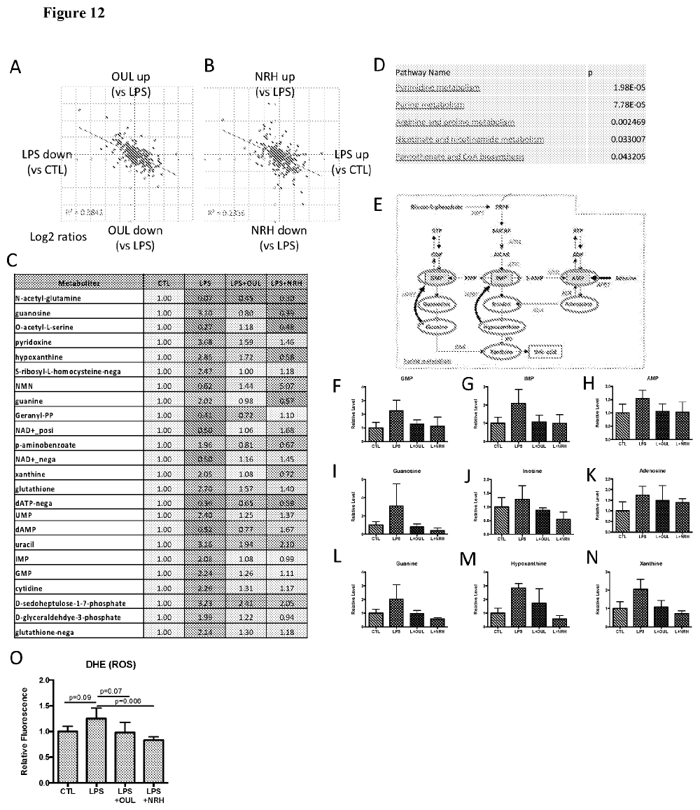

Polar metabolomics on BMDM extracts treated with LPS, OUL35, and NRH were performed. Overall, both drugs appear to blunt the changes induced by LPS (FIG. 12A-B). Interestingly, nucleotide metabolism seems to be particularly responsive to changes NAD⁺ levels (FIG. 12C-N). Recently, Prof. Marcia Haigis (HMS) published that Sirt3, an NAD⁺-sensor, regulates nucleotide metabolism (Gonzalez Herrera et al., 2018). While an increase in nucleotides could promote DNA synthesis, macrophages are reported to suppress the cell cycle in response to LPS. However, macrophages do upregulate reactive oxygen species (ROS) production for bactericidal purposes in response to LPS, and two purines—hypoxanthine and xanthine—are important for this ROS production via the enzyme xanthine oxidase (XO). With the hypothesis that macrophages increase these metabolites for increased ROS production in mind, PARPi and NRH suppress nigericin-induced ROS production, although this experiment must be repeated (FIG. 12O).

### Example 12

### Protecting NAD⁺ Levels Reduces the Severity of LPS-Induced Sepsis

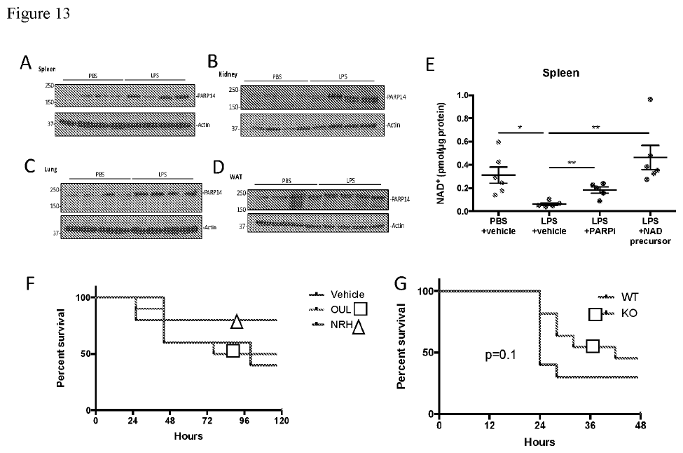

One common assay to measure inflammation in vivo is with LPS induced sepsis, whereby LPS is injected intraperitoneally, resulting in a lethal cytokine storm. Drugs or mutations that suppress inflammation increase survival. Mice euthanized 6 hours after LPS injection show in increase in PARP14 levels in multiple tissues (FIG. 13A-D). NAD⁺ levels fall in multiple tissues, and in at least in spleen, NAD⁺ levels can be protected with a co-injection of PARPi and NRH (FIG. 13E). Mice co-injected with PARPi and NRH show moderately increased survival over mice injected with LPS alone (FIG. 13F), as do PARP14 KO mice (FIG. 13G).

### Example 13

### PARP14 Regulated Falling NAD⁺ Levels During Aging

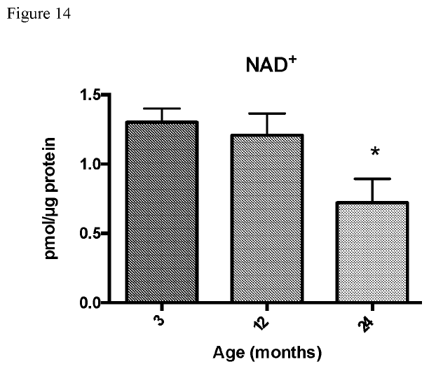

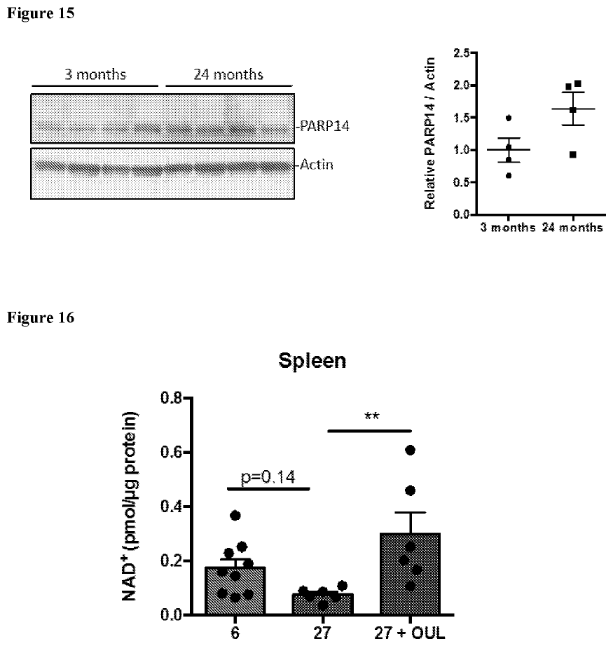

Increased inflammation is a hallmark of aging, and lower NAD⁺ levels have been observed in multiple aged tissues. Previously, it has been shown that NAD⁺ levels drop in peritoneal macrophages during aging (FIG. 14). Recently, A. Brunet's lab published an article on bioRxiv showing that PARP14 is one of only 16 genes that increase in multiple tissues during aging. Please see below.

### INCORPORATION BY REFERENCE

All publications, including but not limited to patents and patent applications, cited in this specification, to the extent that they provide exemplary procedural or other details supplementary to those set forth herein, are specifically incorporated herein by reference as if each individual publication were specifically and individually indicated to be incorporated by reference herein as though fully set forth.

### Equivalents

Those skilled in the art will recognize, or be able to ascertain using no more than routine experimentation, many equivalents of the specific embodiments of the invention described herein. Such equivalents are intended to be encompassed by the following claims.

1. A method for treating or preventing aging, or an aging-related disorder, in a subject in need thereof comprising administering to the subject an effective amount of:
(a) an agent that modulates the level of, activity of, or expression of a poly(ADP-ribose)polymerase 14 (PARP14), or fragment thereof, or a nucleic acid encoding same;
(b) a PARP14 mutant, or fragment thereof, or a nucleic acid encoding same; or (c) both (a) and (b);
to thereby modulate the levels of nicotinamide adenine dinucleotide (NAD⁺) in the subject.

2. A method for treating or preventing a disorder associated with inflammation in a subject in need thereof comprising administering to the subject an effective amount of:
(a) an agent that modulates the level of, activity of, or expression of a PARP14, or a fragment thereof, or a nucleic acid encoding same;
(b) a PARP14 mutant, or fragment thereof, or a nucleic acid encoding same; or (c) both (a) and (b);
to thereby modulate the levels of NAD⁺ in the subject.

3. A method of modulating an inflammatory response in a subject in need thereof comprising administering to the subject an effective amount of:
(a) an agent that modulates the level of, activity of, or expression of a PARP14, or a fragment thereof, or a nucleic acid encoding same;
(b) a PARP14 mutant, or fragment thereof, or a nucleic acid encoding same; or (c) both (a) and (b);
to thereby modulate the levels of NAD⁺ in the subject.

4. A method for increasing stress resistance of a cell comprising introducing into the cell:
(a) an agent that modulates the level of, activity of, or expression of a PARP14, or a fragment thereof, or a nucleic acid encoding same;
(b) a PARP14 mutant, or fragment thereof, or a nucleic acid encoding same; or
(c) both (a) and (b);
to thereby modulate the levels of NAD⁺ in the cell.

5. The method of claim 4, wherein the cell is a mammalian cell, yeast cell, fungal cell, plant cell, or microbial cell.

6. The method of any one of claims 1-5, wherein the agent inhibits the level of, activity of, or expression of the PARP14, or a fragment thereof, or a nucleic acid encoding same.

7. The method of claim 6, wherein the agent inhibits the level of, activity of, or expression of the PARP14, or homologs thereof, as set forth in Table 1.

8. The method of any one of claims 1-5, wherein the PARP14 mutant comprises at least one substitution, mutations, insertion, deletion, or combination thereof, in Macro Domain 1 as set forth in Table 1 or 2.

9. The method of claim 8, wherein the PARP14 mutant comprises at least two, three, four, five, six, seven, eight, nine, ten, or more substitution, mutations, insertion, deletion, or combinations thereof, in Macro Domain 1 as set forth in Table 1 or 2.

10. The method of any one of claims 1-5, wherein the PARP14 mutant comprises at least one substitution, mutations, insertion, or deletion of a phosphorylation site as set forth in Table 1 or 3.

11. The method of any one of claims 1-10, wherein the PARP14 mutant is biologically inactive or functionally defective.

12. The method of claim 11, wherein the PARP14 mutant lacks NADase activity, ADP-ribose hydrolase, mono(ADP-ribosyl)transferase activity, or poly(ADP-ribosyl)transferase activity, or combinations thereof.

13. The method of any one of claims 1-7, wherein the agent is a PARP14 inhibitor.

14. The method of claim 13, wherein the PARP14 inhibitor is a pan-PARP inhibitor.

15. The method of claim 14, wherein the pan-PARP inhibitor is selected from 3-aminobenzamide, KU0058948, BGB-290, Olaparib, ABT-888, CEP-9722, DPQ, NU1025, EB-47, E7016, DiQ, DR2313, 4-ANI, ISQ, 3-hydroxybenzamide, CNQ, 3-AB, PJ34, DPQ, INH2BP, Iniparib, Niraparib (MK-4827), 6(5H)-phenanthridinone, 3-methyl-5-AIQ, Talazoparib, TIQ-A, XAV939, Veliparib, or Rucaparib, or combination thereof.

16. The method of claim 15, wherein the pan-PARP inhibitor is 3-aminobenzimide or PJ-34.

17. The method of claim 13, wherein the PARP14 inhibitor is a Macro Domain 1 inhibitor.

18. The method of claim 17, wherein the Macro Domain 1 inhibitor is selected from NCI-61610 (C₃₄H₂₄N₆O₂), NCI-25457 (C₂₄H₁₆N₂O), NCI-345647_a (C₃₀H₂₆O₁₀), NCI-670283 (C₂₅₄H₂₄O₂), or NCI_127133 (C₂₇H₁₈N₂O₄), or combinations thereof.

19. The method of claim 18, wherein the Macro Domain 1 inhibitor is selected from NSC-61610 or NSC-127-133.

20. The method of any one of claims 1-19, wherein the levels of NAD⁺ are increased.

21. The method of any one of claims 1-19, wherein the agent or PARP14 mutant blocks the fall of NAD⁺ levels in the subject.

22. The method of any one of claims 1-21, wherein the agent or PARP14 mutant increases the level or activity of an enzyme involved in NAD⁺ biosynthesis, an enzymatically active fragment of such an enzyme, a nucleic acid encoding an enzyme involved in NAD⁺ biosynthesis, or an enzymatically active fragment of such a nucleic acid.

23. The method of claim 22, wherein the enzyme is selected from mononucleotide adenylyl transferasel (NMNAT1), NMNAT2, NMNAT3, or nicotinamide phosphoribosyl transferase (NAMPT or NAMPRT).

24. The method of any one of claims 1-23, wherein inflammasome activation is suppressed.

25. The method of any one of claims 1-23, where inflammation is decreased.

26. The method of any one of claims 1-23, where an inflammatory response is depressed or suppressed.

27. The method of claim 1, wherein the aging-related disorder is selected from the group consisting of Alzheimer's disease, diabetes mellitus, heart disease, obesity, osteoporosis, Parkinson's disease, stroke, amniotropic lateral sclerosis, arthritis, atherosclerosis, cachexia, cancer, cardiac hypertrophy, cardiac failure, cardiac hypertrophy, cardiovascular disease, cataracts, colitis, chronic obstructive pulmonary disease, dementia, diabetes mellitus, frailty, heart disease, hepatic steatosis, high blood cholesterol, high blood pressure, Huntington' s disease, hyperglycemia, hypertension, infertility, inflammatory bowel disease, insulin resistance disorder, lethargy, metabolic syndrome, muscular dystrophy, multiple sclerosis, neuropathy, nephropathy, obesity, osteoporosis, Parkinson's disease, psoriasis, retinal degeneration, sarcopenia, sleep disorders, sepsis, and stroke.

28. The method of claim 2, wherein the disorder associated with inflammation is selected from the group consisting of: septic shock, obesity-related inflammation, Parkinson's Disease, Crohn's Disease, Alzheimer's Disease (AD), cardiovascular disease (CVD), inflammatory bowel disease (IBD), chronic obstructive pulmonary disease, an allergic reaction, an autoimmune disease, blood inflammation, joint inflammation, arthritis, asthma, ulcerative colitis, hepatitis, psoriasis, atopic dermatitis, pemphigus, glomerulonephritis, atherosclerosis, sarcoidosis, rheumatoid arthritis, psoriatic arthritis, ankylosing spondylitis, Wegner's syndrome, Goodpasture's syndrome, giant cell arteritis, polyarteritis nodosa, idiopathic pulmonary fibrosis, acute lung injury, post-influenza pneumonia, SARS, tuberculosis, malaria, sepsis, cerebral malaria, Chagas disease, schistosomiasis, bacterial and viral meningitis, cystic fibrosis, multiple sclerosis, encephalomyelitis, sickle cell anemia, pancreatitis, transplantation, systemic lupus erythematosis, autoimmune diabetes, thyroiditis, and radiation pneumonitis, respiratory inflammation, and pulmonary inflammation.

29. The method of any one of claims 1-28, wherein the agent or PARP14 mutant is administered to the subject at a dose of between 0.5-5 grams per day.

30. The method of any of claims 1-29, wherein the agent or the PARP14 mutant is administered in a pharmaceutically effective amount.

31. The method of claim 30, wherein the pharmaceutically effective amount is provided as a pharmaceutical composition in combination with a pharmaceutically-acceptable excipient, diluent, or carrier.

32. The method of any one of claims 1-31, wherein the a) agent is administered simultaneously as the PARP14 mutant, b) agent is administered in combination with PARP14 mutant, c) agent is administered prior to administering the PARP14 mutant, or d) agent is administered subsequently to administering the PARP14 mutant.

33. The method of any of claims 1-32, wherein the subject is a mammal or non-mammal.

34. The method of claim 33, wherein the subject is a human.

35. An agent or PARP14 mutant that increases the level of NAD⁺ for use in treating or preventing aging, or an aging-related disorder.

36. An agent or PARP14 mutant that increases the level of NAD⁺ for use in treating or preventing a disorder associated with inflammation.

37. An agent or PARP14 mutant that increases the level of NAD⁺ for use in modulating an inflammatory response.

38. An agent or PARP14 mutant that increase the level of NAD⁺ for use in increasing stress resistance of a cell.

39. The agent of any one of claims 35-38, wherein the agent inhibits the level of, activity of, or expression of the PARP14, or a fragment thereof, or a nucleic acid encoding same.

40. The agent of any one of claims 35-39, wherein the agent inhibits the level of, activity of, or expression of the PARP14, or homologs thereof, as set forth in Table 1.

41. The agent of any one of claims 35-40, wherein the agent is a PARP14 inhibitor.

42. The agent of claim 41, wherein the PARP14 inhibitor is a pan-PARP inhibitor.

43. The agent of claim 42, wherein the pan-PARP inhibitor is selected from 3-aminobenzamide, KU0058948, BGB-290, Olaparib, ABT-888, CEP-9722, DPQ, NU1025, EB-47, E7016, DiQ, DR2313, 4-ANI, ISQ, 3-hydroxybenzamide, CNQ, 3-AB, PJ34, DPQ, INH2BP, Iniparib, Niraparib (MK-4827), 6(5H)-phenanthridinone, 3-methyl-5-AIQ, Talazoparib, TIQ-A, XAV939, Veliparib, or Rucaparib, or combination thereof.

44. The agent of claim 43, wherein the pan-PARP inhibitor is 3-aminobenzimide or PJ-34.

45. The agent of claim 41, wherein the PARP14 inhibitor is a Macro Domain 1 inhibitor.

46. The agent of claim 45, wherein the Macro Domain 1 inhibitor is selected from NCI-61610 (C₃₄H₂₄N₆O₂), NCI-25457 (C₂₄H₁₆N₂O), NCI-345647_a (C₃₀H₂₆O₁₀), NCI-670283 (C₂₅₄H₂₄O₂), or NCI_127133 (C₂₇H₁₈N₂O₄), or combinations thereof.

47. The agent of claim 46, wherein the Macro Domain 1 inhibitor is selected from NSC-61610 or NSC-127-133.

48. The PARP14 mutant of any one of claims 35-38, wherein said mutant comprises at least one substitution, mutation, insertion, deletion, or combination thereof, in Macro Domain 1 as set forth in Table 1 or 2.

49. The PARP14 mutant of claim 48, wherein said mutant comprises at least two, three, four, five, six, seven, eight, nine, ten, or more substitutions, mutations, insertions, deletions, or combination thereof, in Macro Domain 1 as set forth in Table 1 or 2.

50. The PARP14 mutant of claim 48, wherein said mutant comprises at least one substitution, mutations, insertion, or deletion of a phosphorylation site as set forth in Table 1 or 3.

51. The PARP14 mutant of claim 48, wherein said mutant is biologically inactive or functionally defective.

52. The PARP14 mutant of claim 51, wherein said mutant lacks NADase activity, ADP-ribose hydrolase, mono(ADP-ribosyl)transferase activity, or poly(ADP-ribosyl)transferase activity, or combinations thereof.
